(19) United States
(12) Patent Application Publication
(10) Pub. No.: US 2022/0340863 A1
(43) Pub. Date: Oct. 27, 2022

(54) HYDROGEL COMPOSITIONS COMPRISING PROTIST CELLS

(71) Applicants: THE UNIVERSITY OF MELBOURNE, Melbourne (AU); GRAINS RESEARCH AND DEVELOPMENT CORPORATION, Barton (AU)

(72) Inventors: Helen JACOBE, Melbourne (AU); Ruth Elizabeth HAITES, Melbourne (AU); Sameera SIRISENA, Melbourne (AU)

(73) Assignees: THE UNIVERSITY OF MELBOURNE, Melbourne (AU); GRAINS RESEARCH AND DEVELOPMENT CORPORATION, Barton (AU)

(21) Appl. No.: 17/642,391

(22) PCT Filed: Sep. 11, 2020
(86) PCT No.: PCT/AU2020/050978

§ 371 Date: Mar. 11, 2022

(30) Foreign Application Priority Data
Sep. 13, 2019 (AU) 2019903410

(51) Int. Cl. C12N 1/10; A01N 25/04; C12N 11/04; C12N 11/08; C12N 11/12

(52) U.S. Cl. CPC C12N 1/105; A01N 25/04; C12N 11/04; C12N 11/08; C12N 11/12; C12N 2533/74; C12N 2533/78; C12R 2001/90

(57) ABSTRACT

The present disclosure relates to hydrogels composition comprising protist cells. In particular, the present disclosure relates to hydrogel compositions which may be used to encapsulate or suspend ciliated protist cells, and methods of preparing the same. The present disclosure further relates to methods of infecting molluscs with a ciliated protist cell, and methods and compositions for stabilising ciliated protist cells.

### FIELD OF THE INVENTION

The present disclosure relates to hydrogel compositions comprising protist cells, which are single-celled eukaryotic cells. In particular, the present disclosure relates to a hydrogel composition which may be used to encapsulate or suspend ciliated protist cells, also known as ciliates or ciliate cells, and methods of preparing the same. The present disclosure further relates to methods of infecting or colonising molluscs with a ciliate.

### BACKGROUND OF THE INTRODUCTION

Pests, for example slugs and snails, are a problem in agriculture and horticulture because they damage plants and affect the productivity and quality of crops and plant products. Various strategies have been used to control pest molluscs which include the use of chemical molluscicides (e.g. methiocarb and metaldehyde) which are usually distributed in baits. These chemicals are not just specific for molluscs, and target other animals raising concerns about their toxic effect and environmental contamination.

Biological control agents have been proposed as an alternative to chemical molluscicides. One example is the slug parasitic ciliate Tetrahymena rostrata. T. rostrata ciliates transition from being feeding ciliate cells (called “trophont” cells) to reproductive or resting cyst cells (called “cyst” or “encysted” cells) via a process called “encystment”. The encysted cells then undergo “excystment” to form juvenile cells (called “theront” cells).

Various problems arise associated with using ciliate cells as biological control agents due to their developmental life cycle, including that cells grown in culture media are not robust and/or have limited cell viability so cannot be stored for long periods of time thus are unsuitable for use as pest control agents. There is therefore a need to develop new compositions and methods to enhance the storage, stability, methods for application, and/or parasitic activity of ciliate cells for use in pest control, or at least provide the public with a useful alternative.

### SUMMARY OF THE INVENTION

The present inventors have identified processes for growing and formulating ciliate cells at various developmental stages for production, storage and delivery as a biological control agent for the control of pests, such as molluscs.

In particular, the present inventors have identified that encapsulating or suspending ciliate cells in hydrogels improves the storage, stability and viability of the ciliate cells. The present inventors have also identified that hydrogels can stabilise the ciliate cells encapsulated or suspended therein as either trophont ciliate cells or encysted ciliate cells which remain viable during storage. These cells can be subsequently released from the hydrogel and undergo excystment into theront ciliate cells, which the inventors have also identified can be highly infective to pests such as molluscs. The present invention therefore provides compositions which can be used to store, stabilise and transport viable ciliate cells at different stages in its life cycle, allowing for their use as an effective pest control agent, such as being applied to areas affected or likely to be affected by a pest species.

Accordingly, in a first aspect, there is provided a composition comprising a hydrogel and a population of ciliate cells, wherein the ciliate cells are encapsulated or suspended within the hydrogel, wherein the hydrogel comprises a physically cross-linked hydrogel-forming polymer.

In some embodiments, the ciliate cells are encysted ciliate cells or trophont ciliate cells. Thus advantageously, the compositions of the invention can be used to suspend or encapsulate ciliate cells at various developmental stages. In one embodiment, the ciliate cells are encysted ciliate cells. In another embodiment, the ciliate cells are trophont ciliate cells.

In some embodiments, the hydrogel comprises about 0.1% w/v to about 5% w/v of the hydrogel-forming polymer. In one embodiment, the hydrogel comprises about 0.5% w/v to about 4% w/v of the hydrogel-forming polymer. In another embodiment, the hydrogel comprises about 1% w/v to about 2% w/v of the hydrogel-forming polymer, for example, 1.2% w/v to about 1.7% w/v of the hydrogel forming polymer. In one embodiment, the hydrogel comprises about 1.5% w/v of the hydrogel-forming polymer.

The hydrogel may comprise any suitable hydrogel-forming polymer that is capable of being physically cross-linked. In some embodiments, the hydrogel-forming polymer may be a natural polymer or a synthetic polymer. In some embodiments, the hydrogel-forming polymer may be a homopolymer, copolymer, random copolymer, block copolymer, graft copolymer, and mixtures thereof. In one embodiment, the hydrogel-forming polymer is a natural polymer. In some embodiments, the hydrogel-forming polymer may be a polysaccharide, glycosaminoglycan, or a protein.

In some embodiments, the hydrogel forming polymer is a hydrophilic polymer. For example, the hydrogel may comprise a physical cross-linked hydrophilic polymer.

In one embodiment, the hydrogel-forming polymer is a polysaccharide.

In some embodiments, the hydrogel-forming polymer is selected from one or more of alginate, cellulose, gellan gum, starch, chitin, chitosan, hyaluronan, or carboxymethylcellulose (CMC). In some embodiments, the hydrogel-forming polymer is alginate or carboxymethylcellulose (CMC). In one embodiment, the hydrogel-forming polymer is an alginate, for example sodium alginate. Other hydrogel agents which provide similar characteristics will be employed as equivalents to those disclosed above.

Any physical cross-linking may be suitable in the compositions of the present invention (i.e. ionic, hydrogen-bonding or hydrophobic forces). In one embodiment, the hydrogel-forming polymer is cross-linked via hydrogen bonding or hydrophobic interaction. In another embodiment, the hydrogel forming polymer is ionically cross-linked. Any suitable ionic cross-linker can be used in the compositions of the present invention, for example a polyvalent cation. In one embodiment, the hydrogel-forming polymer is ionically cross-linked by a polyvalent cation. In some embodiments, the polyvalent cation may be a divalent cation, a trivalent cation or a mixture thereof. In one embodiment, the polyvalent cation is a divalent cation. In another embodiment, the polyvalent cation is a trivalent cation. In some embodiments, the polyvalent cation comprises both divalent and trivalent cations. In some embodiments, the hydrogel-forming polymer is ionically cross-linked by a divalent cation or trivalent cation selected from one or more of Ca²⁺, Mg²⁺, Sr²⁺, Ba²⁺, Zn²⁺, Be²⁺ Fe³⁺, Al³⁺, or Mn³⁺. In one embodiment, the hydrogel-forming polymer is ionically cross-linked by a divalent cation selected from one or more of Ca²⁺, Mg²⁺, Sr²⁺, Ba²⁺, Zn²⁺, or Be²⁺. In one embodiment, the hydrogel-forming polymer is ionically cross-linked by Ca²⁺.

In one embodiment, the composition further comprises magnesium sulfate.

In some embodiments, the hydrogel comprises a plurality of hydrogel beads, wherein one or more of the hydrogel beads encapsulates one or more of the ciliate cells. Trophont ciliate cells encapsulated within hydrogel beads undergo encystment to form encysted ciliate cells, and remain as encysted ciliate cells within the hydrogel bead.

In some embodiments, the hydrogel beads have an average size of about 100 μm (0.1 mm) to about 5 mm in diameter.

In some embodiments, the hydrogel further comprises an attractant or feeding stimulant. The attractant may be a nutrient source or a pheromone. The feeding stimulant may be a plant extract. In one embodiment, the attractant is a nutrient source. In some embodiments, the attractant may be provided as an outer coating on the hydrogel.

Alternatively, in another embodiment, the attractant may be provided as a separate component in the composition (e.g. forms part of a carrier which the hydrogel may be dispersed in).

In some embodiments, the average number of ciliate cells encapsulated in the one or more hydrogel beads is about 100 to about 10,000 ciliate cells per bead. For example, in one embodiment, the average number of ciliate cells encapsulated in the one or more hydrogel beads is about 1000 ciliate cells per bead.

In some embodiments, the ciliate cells encapsulated or suspended in the hydrogel remain viable for at least about five weeks. In some embodiments, the ciliate cells encapsulated or suspended in the hydrogel remain viable for at least 24 weeks. For example, ciliate cells encapsulated or suspended in the hydrogel remain viable and stable as trophonts or cysts and upon release from the hydrogel undergo excystment into theront ciliate cells.

In a second and related aspect, there is provided a method of encapsulating or suspending a population of ciliate cells within a hydrogel, the method comprising:

a) adding a suspension of ciliate cells to a hydrogel-forming polymer solution to form a hydrogel,

wherein the ciliate cells are encapsulated or suspended by the hydrogel.

In one embodiment, step a) comprises adding a suspension of ciliate cells to a hydrogel-forming polymer solution and an ionic cross-linker solution to form a hydrogel, wherein the ciliate cells are encapsulated or suspended by the hydrogel.

In one embodiment, the ciliate cells in step a) are trophont ciliate cells.

The present inventors have identified that, in some embodiments, depending on the type of hydrogel, the trophont ciliate cells are encapsulated within the hydrogel and migrate to the centre of the hydrogel and encyst to form encysted ciliate cells, or they distribute evenly throughout the hydrogel and remain suspended as trophont ciliate cells. Therefore, in one embodiment, trophont ciliate cells are encapsulated by the hydrogel and undergo encystment within the hydrogel to form one or more encysted ciliate cells. In another embodiment, trophont ciliate cells are suspended within the hydrogel and remain as trophont ciliate cells.

In another embodiment, the ciliate cells in step a) are pre-formed encysted ciliate cells. The present inventors have identified that, in some embodiments, pre-formed encysted cells remain stable and viable when suspended or encapsulated within the hydrogel.

In one embodiment, the method further comprises the step a1) preparing a mixture comprising the suspension of ciliate cells and the hydrogel-forming polymer solution and adding the mixture of a1) to the cross-linker solution to form the hydrogel. In a further embodiment, one or more droplets of the mixture of step a1) are added to the cross-linker cation solution to form the hydrogel.

In one embodiment, step a) or step a1) further comprises magnesium sulfate. In some embodiments, the concentration of the magnesium sulfate is about 20 μM to about 100 μM.

In some embodiments, the suspension of ciliate cells and the hydrogel-forming polymer solution is exposed to the cross-linker solution for less than about 20 minutes. In some embodiments, the suspension of ciliate cells and the hydrogel-forming polymer solution is exposed to the cross-linker solution for about 1 minute to about 10 minutes. For example, in one embodiment, the suspension of ciliate cells and the hydrogel-forming polymer solution is exposed to the cross-linker solution for about 5 minutes.

In some embodiments, the density of ciliate cells in the suspension of ciliate cells is at least about 1×10⁵ cells/mL. In some embodiments, the density of ciliate cells in the suspension of ciliate cells is at least about 1×10⁵, 1×10⁶, 1×10⁷, 1×10⁸, 1×10⁹ or 1×10¹⁰ cells/mL. In one embodiment, the density of ciliate cells in the suspensions of ciliate cells is about 1×10⁵ cells/mL to about 1×10⁹ cells/mL.

In some embodiments, the hydrogel-forming polymer in the hydrogel-forming polymer solution has a concentration of about 0.1% w/v to about 5% w/v. For example, in one embodiment, the hydrogel-forming polymer solution has a concentration of about 1.5% w/v.

In one embodiment, the vol:vol ratio of the suspension of ciliate cells to the hydrogel-forming polymer solution is about 1:4.

The hydrogel-forming polymer solution may comprise any suitable hydrogel-forming polymer than is capable of being physically cross-linked. In some embodiments, the hydrogel-forming polymer solution may be a natural polymer or a synthetic polymer.

In some embodiments, the hydrogel-forming polymer may comprise be a homopolymer, copolymer, random copolymer, block copolymer, graft copolymer, and mixtures thereof. In one embodiment, the hydrogel-forming polymer solution comprises a polysaccharide. In some embodiments, the hydrogel-forming polymer solution comprises one or more of alginate, cellulose, gellan gum, starch, chitin, chitosan, hyaluronan or carboxymethylcellulose (CMC). In some embodiments, the hydrogel-forming polymer solution comprises alginate or carboxymethylcellulose (CMC). In one embodiment, the hydrogel-forming polymer solution comprises alginate. In one embodiment, the alginate is sodium alginate.

Any physical cross-linking may be suitable to cross-link the hydrogel-forming polymer (i.e. ionic, hydrogen-bonding or hydrophobic forces). In one embodiment, the hydrogel-forming polymer is cross-linked via hydrogen bonding or hydrophobic interaction. In another embodiment, the hydrogel forming polymer is ionically cross-linked by an ionic cross-linker solution. Any suitable cross-linker capable of cross-linking the hydrogel-forming polymer can be used to prepare the compositions of the present invention. In one embodiment, the cross-linker solution may comprise polyvalent cations. In one embodiment, the hydrogel-forming polymer solution is ionically cross-linked by a polyvalent cation. Therefore, in some embodiments, the cross-linker solution comprises polyvalent cations. In some embodiments, the polyvalent cations in the cross-linker solution is about 20 mM to about 500 mM. For example, in one embodiment, the concentration of the polyvalent cations in the cross-linker solution is about 50 mM.

In some embodiments, the polyvalent cations in the cross-linker solution may be divalent cations, trivalent cations or a mixture thereof. For example, in one embodiment, the polyvalent cations in the cross-linker solution are divalent cations. In another embodiment, the polyvalent cations in the cross-linker solution are trivalent cations. In some embodiments, the polyvalent cations in the cross-linker solution comprise both divalent and trivalent cations.

In some embodiments, the cross-linker solution comprises divalent cations or trivalent cations selected from one or more of Ca²⁺, Mg²⁺, Sr²⁺, Ba²⁺, Zn²⁺, Be²⁺ Fe³⁺, Al³⁺ or Mn³⁺. In one embodiment, the cross-linker solution comprises divalent cations selected from one or more of Ca²⁺, Mg²⁺, Sr²⁺, Ba²⁺, Zn²⁺, or Be²⁺. In one embodiment, the cross-linker solution comprises Ca²⁺ cations. In one embodiment, the cross-linker solution is calcium chloride (CaCl₂). In another embodiment, the cross-linker solution comprises Fe³⁺ cations. In one embodiment, the cross-linker solution is iron (III) phosphate (FePO₄) or iron (III) chloride (FeCl₃).

In some embodiments, the method produces a hydrogel in the form of a plurality of hydrogel beads. In some embodiments, the ciliate cells are located in the centre of the hydrogel beads. In some embodiments, the hydrogel beads have an average size of about 1 mm to about 5 mm in diameter.

In one embodiment, the method further comprises the step b) washing the formed hydrogel to remove any excess cross-linker solution. In a further embodiment, the method further comprises the step c) storing the washed hydrogel in a sealed container. In one embodiment, the hydrogel is stored in the dark. In one embodiment, the hydrogel is stored at about 4° C. to about 28° C.

In a related and third aspect, there is provided a method of inducing the encystment of ciliate cells, the method comprising incubating a population of trophont ciliate cells in a buffer solution comprising magnesium ions, wherein the trophont ciliate cells undergo encystment to form one or more encysted ciliate cells. In one embodiment, the buffer solution comprises magnesium sulfate. The present inventors have identified that, in some embodiments, a buffer solution comprising magnesium ions can trigger encystment of trophont ciliate cells.

In some embodiments, the trophont ciliate cells are incubated in the buffer solution at a temperature of about 20 to 30° C.

In some embodiments, the trophont ciliate cells are incubated in the buffer solution for about 12 to 48 hours.

In some embodiments, the concentration of magnesium ions in the buffer solution is about 15 μM to about 500 μM.

Any ciliate cell capable of undergoing encystment to form encysted ciliate cells may be used in the compositions and methods of the present invention (e.g. ciliate cells that can form trophont ciliate cells or encysted ciliate cells). For example, in one embodiment, the ciliate cells are any member of the Ciliophora phylum. In some embodiments, the ciliate cells are a member of the Heterotrichea, Karyorelictea, Armophorea, Litostomatea, Colpodea, Nassophorea, Phyllopharyngea, Prostomatea, Plagiopylea, Oligohymenophorea, Protocruziea, Spirotrichea, or Cariotrichea class. In some embodiments, the ciliate cells are a member of the Apostomatia, Astomatia, Hymenostomatia, Peniculia, Peritrichia, or Scuticociliatia order. In some embodiments, the ciliate cells are a member of the Tetrahymenidae, Ophryoglenina, or Peniculina family.

In one embodiment, the ciliate cells are a member of the Tetrahymena genus. In some embodiments, the ciliate cells are of the T. rostrata, T. hegewischi, T. hyperangularis, T. malaccensis, T. patula, T. pigmentosa, T. pyriformis, T. thermophila, T. vorax, T. geleii, T. corlissi, T. empidokyrea or T. limacis species. In some embodiments, the ciliate cells are of the T. rostrata, T. corlissi, or T. empidokyrea species. In one embodiment, the ciliate cells are of the T. rostrata species. Other species of Tetrahymena are also envisaged.

In a related and fourth aspect, there is provided an isolated strain of T. rostrata which has one or more or all of the following features:

i) deposited under PTA-126056 on 13 Aug. 2019 at the American Type Culture Collection,

ii) comprises a mitochondrial genome which has a nucleotide sequence as shown in SEQ ID NO:1 or a sequence at least 90% identical thereto, and

iii) comprises a cox1 gene which has a nucleotide sequence as shown in SEQ ID NO:7 or a sequence at least 99% identical thereto.

In related and fifth aspect, there is provided a composition comprising the T. rostrata strain, and one or more acceptable carriers.

In a related and sixth aspect, there is provided a method of infecting or colonising a pest species with a ciliate, the method comprising applying to an area affected or likely to be affected by a pest species one or more of a hydrogel composition according to the first aspect, a hydrogel composition or encysted ciliate cells prepared by the method according to the second or third aspect, a strain of T. rostrata according to the fourth aspect or the composition according to the fifth aspect.

In some embodiments, the method comprises adding the hydrogel with a solution to disrupt the ionic cross-linking in the hydrogel prior to applying the hydrogel to the area. In one embodiment, the solution that disrupts the cross-linking in the hydrogel is water, citrate buffer solution, or an alginate lyase solution.

In some embodiments, the method results in the ciliate killing or affecting the fitness of the pest species.

Any pest species that can be infected or colonised by ciliate cells are suitable for infecting or colonising with the hydrogel composition, ciliate cells and/or strains of the present invention. In some embodiments, the pest species is an invertebrate. The invertebrate may be a mollusc or an arthropod, such as a dipteran (e.g. as a mosquito). In some embodiments, the pest species is a mollusc. In some embodiments, the mollusc is a Gastropod. In some embodiments, the Gastropod is a snail or slug.

In a related and seventh aspect, there is provided a method of inducing the encystment of ciliate cells, the method comprising incubating a population of trophont ciliate cells in an aqueous solution comprising suspended soil particles, wherein the trophont ciliate cells undergo encystment to form one or more encysted ciliate cells.

In a related and eighth aspect, there is provided a method of stabilising encysted ciliate cells, the method comprising dehydrating an aqueous solution comprising a population of encysted ciliate cells and suspended soil particles.

In a related and ninth aspect, there is provided composition for stabilising encysted ciliate cells, the composition comprising encysted ciliate cells suspended in a buffer solution comprising magnesium ions.

Any embodiment herein shall be taken to apply mutatis mutandis to any other embodiment unless specifically stated otherwise. For instance, the skilled person would understand that examples of ciliate cells and/or hydrogel-forming polymers outlined above for the hydrogel compositions equally apply to the methods of encapsulating or suspending a population of ciliate cells, methods of inducing encystment and/or methods of infecting a pest species.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF FIGURES

FIG. 1—Ciliate cell developmental stages: Schematic diagram of the main developmental stages of the ciliate T. rostrata depicting 1) trophont ciliate cells, 2) encysted ciliate cells and 3) theront ciliate cells. Trophont ciliate cells undergo “encystment” to form encysted ciliate cells (or “cysts”). Encysted ciliate cells undergo “excystment” to form theront ciliate cells. Theront ciliate cells undergo “maturation” to form trophont ciliate cells.

FIG. 2—T. rostrata TRAUS strain closely related to other T. rostrata strains: MrBayes tree and nucleotide alignment showing the relationship between 689 bp barcode region of the cox1 gene sequences derived from strains of T. rostrata. T. rostrata TRAUS cox1 was. 98.7% identical to TR 1016 and TR 1015 and 95.7-95.8% identical to TRO1, TRO2, TRO3, TR 1035 and TR 1034 indicating that they are all the same species.

FIG. 3A—Encysted ciliate cells are unstable and excyst using buffered soil infusion methods at 20° C. and remain encysted at 26° C.: T. rostrata TRAUS trophonts were moved from nutrient rich media into starvation media to induce encystment. Cells were starved in buffered soil infusion at 20° C. (light grey) and 26° C. (dark grey). The proportion of round cells were counted. Giemsa staining and morphology confirmed that the round cells were cysts so that data is expressed as the % cysts. Three or more individual cultures were sampled for each point and the error bars represent the maximum and minimal values. Spontaneous excystment was observed after seven days at 20° C. and this continued, so by 35 days only 10% of the cells were encysted cells (light grey) highlighting the unstable nature of encysted cells generated using buffered soil infusion methods at 20° C. At 26° C., 84-93% of the cells were cysts after 24 hours and no spontaneous excystment occurred during 35 days of observation (dark grey).

FIG. 3B—Effect of pre-culture media on encystment in soil infusion buffer: Percent of trophonts that formed cysts in soil infusion buffer at 26° C. after culture in RM9, PPYE or PP media.

FIG. 3C—Maturation of cysts formed via encystment in soil infusion buffer: Transmission electron microscope images of cross sections of T. rostrata cysts A) mucocysts discharging and remnants of ciliate present, B) development of cysts wall and C) mature cyst capsule.

FIG. 3D—Effect of soil particle size on encystment in soil infusion buffer: Cyst formation in HEPES buffer with different sizes of pine bark particles used at 0.1% w/v or 0.01% w/v.

FIGS. 4A to 4E—Theront ciliate cells are more infective than trophont ciliate cells: A) Mortality of D. reticulatum exposed to T. rostrata trophonts or theronts. 5 slugs/tube, 10 replicates for trophonts, 6 replicates for theronts. 1.8×10⁴ theronts/tube, 1.0×10⁴ trophonts/tube. The percent mortality was corrected for any deaths. Theronts killed slugs faster than trophonts. B) Mortality of D. reticulatum exposed to live and heat-killed T. rostrata theronts. C) Top: Logit(P) mortality 4 slugs per tube vs log 10 number of T. rostrata per tube. Bottom: Probit P mortality 5 slugs per tube vs log 10 number of T. rostrata per tube. D) LD₅₀ calculated by Logit and Probit vs the end of the day that data was collected after first exposure to T. rostrata. This indicates that a higher dose kills 50% quicker than a lower dose. i.e. ˜10,000 takes ≤7 days whereas ˜500 takes 14-21 days E) Percent mortality of D. reticulatum at 7, 14 and 21 days after exposure to T. rostrata theronts in relation to dose.

FIG. 5A—Ciliate cells suspended within hydrogels remained stable after 4 weeks: Ciliate cell viability and morphology were observed by microscopy after 1 week (W1), 2 weeks (W2), 3 weeks (W3), and 4 weeks (W4). Cells retained their trophont shape, and were evenly dispersed and were not adhered to any surfaces or sedimented at the bottom. The subculturing showed that the cells stored in carboxymethylcellulose (CMC) at 4° C. were viable and readily multiplied in fresh media compared to the controls in media.

FIG. 5B—Ciliate cells suspended within core-shell hydrogel beads remained stable: Micrographs of CMC-alginate core-shell hydrogel beads comprising trophonts suspended within the CMC core. A) Hardened alginate-carboxymethylcellulose core-shell beads with trophonts incubated in PPYE nutrient medium showing large numbers of motile trophonts and B) Hardened alginate-carboxymethylcellulose core-shell beads with trophonts incubated in 10 mM HEPES pH7 buffer showing no multiplication.

FIG. 6A—Trophont cells migrate to centre of hydrogel beads during cross-linking: Solid alginate hydrogel beads encapsulating A) trophont ciliate cells (dark centres) and B) encysted ciliate cells.

FIG. 6B—Morphology of alginate hydrogels encapsulating ciliate cells: Alginate hydrogel beads with encapsulated T. rostrata cells. Alginate beads produced measured approximately 3 mm in diameter. The centre where the cells are concentrated is visible.

FIG. 7—Encysted ciliate cells encapsulated within alginate hydrogels remain viable and stable after 1 week storage: Cells in alginate beads after 1 week storage at 20° C. Cells pictured by cutting open the alginate bead on a microscope slide with a drop of water. A-C) Magnification×100, D-E) Magnification×400. A-B shows cells released into the surrounding water on the microscope slide. C shows the concentration of cells typically seen in the centre of an alginate bead. In D and E, the cells were rotating inside of the thick cyst wall indicating viability.

FIG. 8—Encysted ciliate cells encapsulated within alginate hydrogels remain viable and stable after 4 weeks storage: Cells in alginate beads after 4 weeks storage at 20° C. Cells pictured by cutting open the alginate bead on a microscope slide with a drop of water. A) Magnification×100, B-E) Magnification×400. In B and C, the cells were rotating inside the thick cyst wall indicating viability.

FIG. 9—Encysted ciliate cells encapsulated within alginate hydrogels remain viable and stable after 11 weeks: Encysted ciliate cells encapsulated within alginate hydrogels were released from alginate gel beads after 11 weeks. Top) Pre-formed encysted cells made in soil infusion and then encapsulated in the hydrogel and Bottom) encysted cells formed via in-gel encystment from trophont cells. All cells were still cysts and could be stimulated to start moving around within the cyst coat indicating viability.

FIG. 10—Encapsulated encysted ciliate cells excyst into theronts when released from hydrogels: Giemsa stained cells harvested from 4 week old alginate beads. A-D (Magnification ×400). The stained nuclei showed the characteristic butterfly effect of theront ciliate cells, as compared to the defined macro and micro nuclei in a trophont.

FIG. 11—Growth of ciliate cells released from hydrogels: Comparison of growth curves for alginate beads with week's 1-4 storage life cultured in PPYE at 20° C. Cells demonstrated normal growth patterns highlighting good cell viability during storage.

FIG. 12A—Magnesium sulfate induces encystment of T. rostrata: Encystment in various concentrations of MgSO₄ at A) 26° C. and B) 20° C. respectively on day 1 (dark grey) and day 6 (light grey) of the incubation. The y-axis is the percent of cells with round cyst morphology in 3 independent cultures (n=3). The bars show the maximum and minimum values.

FIG. 12B—Magnesium sulfate stabilises pre-formed cysts: Survival of T. rostrata TRAUS soil infusion buffer cysts exposed to 0 (light grey) and 25 mM (dark grey) MgSO4. MPN/ml and the proportion of round, cyst cells were determined at 0, 3, 7, 14 and 27 days from 3 separate cultures. The 95% confidence intervals are shown.

FIG. 12C—Encysted ciliate cells tolerate dehydration: Dark Grey) Encysted ciliate cells remain encysted after 18 days following dehydration at a relative humidity of less than 75.5% highlighting that dehydration can stabilise encysted ciliate cells. Light grey) Trophonts suspended in soil infusion buffer (SI-H) encysted during dehydration at a relative humidity of less than 75.5% highlighting that dehydration can induce encystment of trophont ciliate cells. Grey) Trophonts suspended in buffer alone (H) did not encyst when dehydrated. The percent of cyst cells in the culture were measured and plotted. The 95% confidences are shown for the maximum probable number (MPNs) and maximum and minimum values for the percent of cyst cells.

FIG. 13—Survival curves of slugs exposed to theronts: A) Survival curves of slugs exposed to theronts encysted in buffered aqueous solution comprising composted pine bark particles (CI) over 7 days, with no refuge (Experiment 1). Mortality of slugs exposed to theronts was significantly different to the control group (P<0.002). B and C) Survival curves of slugs exposed to theronts encysted in buffered aqueous soil solution comprising soil infusion containing pine bark particles (SI) over 7 days with no refuge (D: Experiment 1; e) Experiment 3). Mortality of slugs exposed to theronts was significantly different from that of the control group (D: (P<0.003) and E: (P>0.0002). Statistical analysis performed was log-rank test with GraphPad Prism.

FIG. 14—Theront infected slugs display superior tentacle impairment: Comparison of slugs displaying superior tentacle impairment. Healthy slug not exposed to theronts. Mild moderate and severely impaired slugs all exposed to theronts.

FIGS. 15, 16 and 17—Theront infected slugs display ocular difficulties and/or death: Heat map of behaviour of slugs exposed to theronts or medium control in Experiment 1 (FIG. 15), Experiment 2 (FIG. 16), and Experiment 3 (FIG. 17). Green indicated healthy status, orange ocular difficulties and red death. M the slug was not observed.

FIG. 18—Ciliates found in slug renal tissue after exposure to T. rostrata: Histological sectioning of slug renal tissue from multiple slugs taken with a Leica light microscope 40× or 400× magnification. Images A to J display slugs exposed to T. rostrata. A) shows ciliates free swimming within the saccular portion of the renal tissue along with several ciliates encapsulated in granulomas. B) Ciliates encapsulated in granuloma structures. C) Many free-swimming cells in the renal tissue. D) Ciliates actively dividing in the renal tissue. E and F). Ciliates free-swimming in the renal tissue and grazing on vacuola cells. G and H) Saccular portion of the renal tissue either side of the pulmonary cavity filled with ciliates. I and J). Saccular portion of the renal tissue of a control slug, showing healthy renal tissue structure.

FIG. 19—Ciliates found in slug heart after exposure to T. rostrata: Histological sectioning of pulmonary region. Images taken on a Leica light microscope 40× or 400×magnification. A) shows the pulmonary region of a healthy slug. B and C) show a single ciliate in the heart and an enlarged chamber of the heart of a slug exposed to T. rostrata.

FIG. 20—Ciliates found in slug muscle after exposure to T. rostrata: Histological sectioning of slugs taken with a Leica light microscope 40× or 400×magnification. A to D) These images show ciliates in the muscle tissue between the skin and body cavity of the slug.

FIG. 21—Ciliates found in the slug interstitial space after exposure to T. rostrata: Histological sectioning images taken with a Leica light microscope 40× or 400×magnification. A and B) show a single ciliate by the developing gonad.

FIG. 22—Ciliates found in slug arteries after exposure to T. rostrata: Histological sectioning of slugs taken with a Leica light microscope 40× or 400×magnification. a) and b) show a single ciliate in an artery of the slug.

FIG. 23—Slugs found to have tumour structures after exposure to T. rostrata: Histological sectioning of slugs taken with a Leica light microscope 40×magnification. A) and B) show the formation of tumour like structures in the pulmonary cavity of the slugs. These structures are formed from the epicardial cells of the heart and renal tissue. C) shows aggregating hypertrophic amoebocytes within the pulmonary cavity.

FIG. 24—Trophonts of T. rostrata TR01 and TRAUS kill adult slugs: Mortality of adult D. reticulatum exposed to trophonts of T. rostrata TR01 (light grey) and TRAUS (dark grey). 15 replicates of groups of 3 slugs were exposed to 5000 T. rostrata. The containers were held at 16° C.

FIG. 25—Neonates of D. reticulatum exposed to TRO1 and TRAUS trophonts die more quickly than adults: Mortality of neonates of D. reticulatum exposed to T. rostrata TRO1 and TRAUS trophonts. 16° C. 6 groups of 10 slugs for TRO1 (light grey) and TRAUS (dark grey). 4 groups of 10 slugs for the untreated controls.

FIG. 26—Theronts of T. rostrata kill A. valentianus and L. flavus slugs: Percent mortality of A. valentianus (black, dark grey and grey) and L. flavus (light grey) exposed to T. rostrata theronts.

### KEY TO THE SEQUENCE LISTING

SEQ ID NO:1—Mitochondrial genome of T. rostrata strain TRAUS isolated from Deroceras reticulatum.
SEQ ID NO:2—Cox1 open reading frame from T. rostrata strain 1034.
SEQ ID NO:3—Cox1 open reading frame from T. rostrata strain 1035.
SEQ ID NO:4—Cox1 open reading frame from T. rostrata strain TRO1.
SEQ ID NO:5—Cox1 open reading frame from T. rostrata strain TRO3.
SEQ ID NO:6—Cox1 open reading frame from T. rostrata strain TRO2.
SEQ ID NO:7—Cox1 open reading frame from T. rostrata strain TRAUS.
SEQ ID NO:8—Cox1 open reading frame from T. rostrata strain 1015.
SEQ ID NO:9—Cox1 open reading frame from T. rostrata strain 1016.

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., in hydrogel formulation, pest control, molecular genetics, and ciliate physiology).

As used herein, the term “about”, unless stated to the contrary, refers to +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “consists of”, or variations such as “consisting of”, refers to the inclusion of any stated element, integer or step, or group of elements, integers or steps, that are recited in context with this term, and excludes any other element, integer or step, or group of elements, integers or steps, that are not recited in context with this term.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “suspended” in the context of ciliate cells being suspended within the hydrogel, refers to a hydrogel that can flow (i.e. not a rigid structure) but is sufficient to hold the ciliate cells in suspension. The ciliate cells can migrate throughout the hydrogel while remaining suspended.

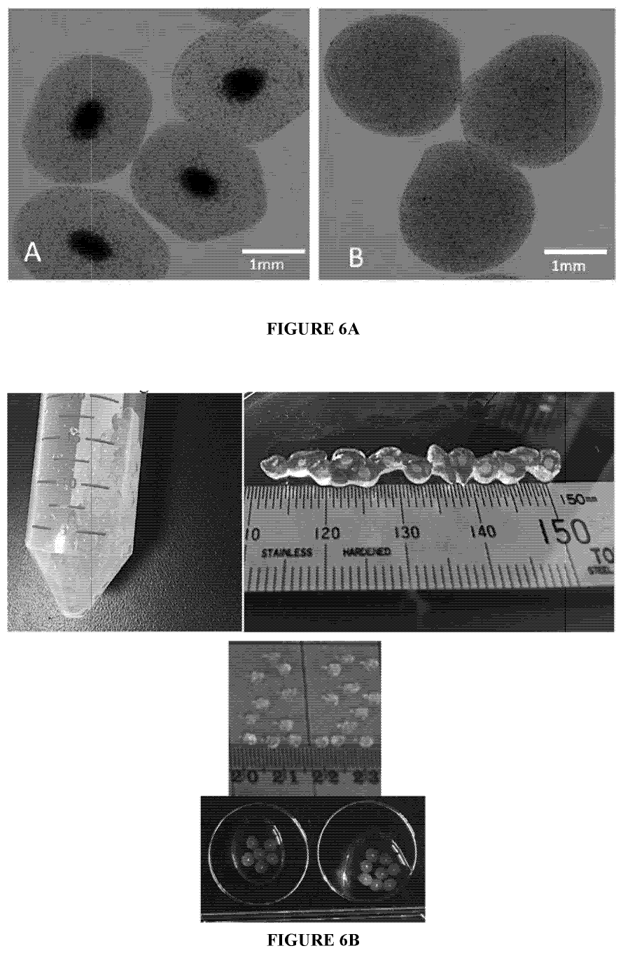

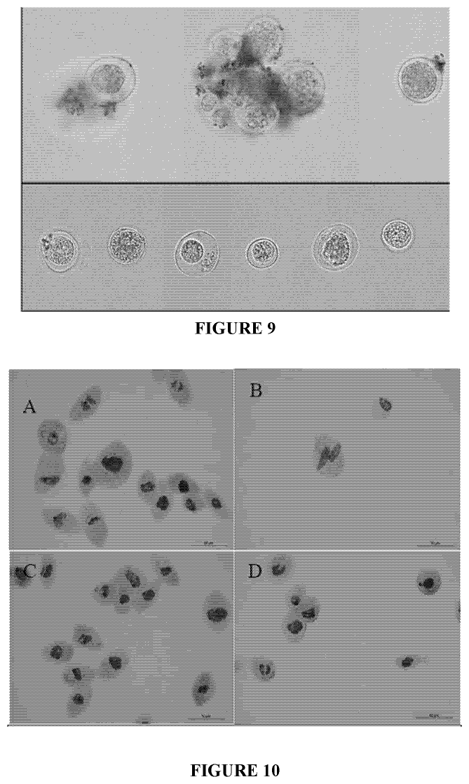

The term “encapsulated” in the context of ciliate cells being encapsulated within the hydrogel refers to the trapping of ciliate cells within the hydrogel. For example, the ciliate cells are trapped within a piece of the hydrogel with defined edges, e.g. within a bead. For example, some ciliate cells encapsulated within a hydrogel bead can still migrate within the bead but are essentially confined by the gelled wall of the hydrogel bead i.e. encapsulated. In contrast, other ciliate cells suspended in a hydrogel remained dispersed throughout the entire hydrogel. An example of ciliate cells encapsulated within a hydrogel can be seen in FIGS. 6 and 9.

### Ciliate Cells

The methods of the present invention can be used to encapsulate and/or suspend ciliated protist cells, also known as ciliates or ciliate cells, in a hydrogel. The term “ciliates” refers to a group of protozoans characterized by the presence of hair-like organelles called cilia. It is the presence of cilia which distinguishes ciliate cells from other protist cells.

The developmental stage cycle of ciliate cells can be separated into three main stages, namely the formation of 1) “trophont” ciliate cells (also known as trophozoites), 2) “encysted” or “cyst” ciliate cells and 3) “theront” ciliate cells. Cells go through several stages of development during autogamy and cyst maturation.

Briefly, the trophont cells are at the growing and feeding stage, encysted cells are the reproductive or resting stage, and theront cells are excysted cells.

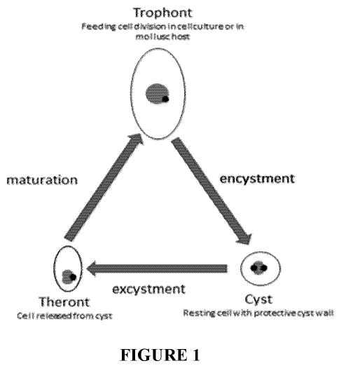

A simple summary of the life cycle of ciliate cells is provided in FIG. 1, where trophont cells undergo a process called “encystment” to form encysted cells. The encysted cells can then undergo a process called “excystment” to form theront cells. The developmental cycle closes where the theront cells mature (i.e. “maturation”) to form trophont cells. The encystment of trophont ciliate cells can be induced by various external stimuli such as starvation and cell aggregation. For example, trophonts respond to encystment stimuli by transforming into small, rapid swimming pre-cystic cells and then round up and secrete large amounts of mucin which condenses and gradually forms a laminar cyst wall which develops into the hick wall of encysted ciliate cells.

The present inventors have identified that, in some embodiments, hydrogel compositions can encapsulate and/or suspend ciliate cells. In some embodiments, the hydrogel compositions can encapsulate and/or suspend encysted ciliate cells, which remain stable and viable and do not excyst into theront ciliate cells. In other embodiments, trophont ciliate cells can also be suspended and/or encapsulated within the hydrogel and either remain as trophont ciliate cells or undergo encystment within the hydrogel to form encysted ciliate cells. In another embodiment, theront ciliate cells may be suspended and/or encapsulated within the hydrogel.

Accordingly, it will be appreciated that the hydrogel compositions according to the present disclosure may suspend or encapsulate any ciliate cell.

In some embodiments, the ciliate cells encapsulated or suspended within the hydrogel may be encysted ciliate cells or trophont ciliate cells. In one preferred embodiment, the ciliate cells are encysted ciliate cells. In an alternative embodiment, the ciliate cells are trophont ciliate cells. In some embodiments, the ciliate cells may be a mixture of trophont ciliate cells or encysted ciliate cells. In a further embodiment, the trophont ciliate cell may undergo encystment within the hydrogel to form an encysted ciliate cell. The present inventors have found that, in some embodiments, encapsulating or suspending ciliate cells (e.g. trophont or encysted ciliate cells) within a hydrogel composition resulted in improved stability and that the ciliate cells also remained viable for a longer period of time.

The hydrogel compositions may suspend or encapsulate a population of ciliate cells. For example, the ciliate cells may be any member of the Ciliophora phylum.

In one preferred embodiment, the ciliate cells are cells that are capable of encystment. The specific type of ciliate cell that is used in the hydrogel composition may also depend on a number of variables, including but not limited to, the type of hydrogel in the composition, the area to be treated with the hydrogel composition, the soil type the hydrogel composition is being dispersed in, and/or the pest species being targeted for pest control (e.g. the type of pest, such as a Gastropod).

In some embodiments, the ciliate cell is a member of the Intramacronucleata, Ventrata, Spirotrichia, or Rhabdophora subphylum. In one preferred embodiment, the ciliate cell is a member of the Intramacronucleata subphylum.

In some embodiments, the ciliate cell is a member of Heterotrichea, Karyorelictea, Armophorea, Litostomatea, Colpodea, Nassophorea, Phyllopharyngea, Prostomatea, Plagiopylea, Oligohymenophorea, Protocruziea, Spirotrichea, or Cariotrichea class. In a preferred embodiment, the ciliate cells are a member of the Oligohymenophorea class.

In some embodiments, the ciliate cells are a member of the Apostomatia, Astomatia, Hymenostomatia, Peniculia, Peritrichia, or Scuticociliatia order. In a preferred embodiment, the ciliate cells are a member of the Hymenostomatia order.

In some embodiment, the ciliate cells are a member of the Tetrahymenina, or Ophryoglenia sub order. In one embodiment, the ciliate cells are a member of the Tetrahymenina sub order. In another embodiment, the ciliate cells are a member of the Ophryoglenia sub order.

In some embodiments, the ciliate cells are a member of the Tetrahymenidae, Ophryoglenina, or Peniculina family.

In some embodiments, the ciliate cells are a member of the Tetrahymena or Lambornella genus. In one embodiment, the ciliate cells are a member of the Lambornella genus. In a preferred embodiment, the ciliate cells are a member of the Tetrahymena genus.

In some embodiments, the ciliate cells are of the T. rostrata, T. hegewischi, T. hyperangularis, T. malaccensis, T. patula, T. pigmentosa, T. pyriformis, T. thermophila, T. vorax, T. geleii, T. corlissi, T. empidokyrea, T. rotunda, or T. limacis species. In a preferred embodiment, the ciliate cells is of the T. rostrata species. In other embodiments, the ciliate cells are of the Lambornella clarki species.

### Compositions Comprising a Hydrogel and Ciliate Cells

The present invention provides a composition comprising a hydrogel and a population of ciliate cells, wherein the ciliate cells are encapsulated or suspended within the hydrogel. In one embodiment, the hydrogel comprises a physically cross-linked hydrogel-forming polymer.

The term “hydrogel” refers to a substance formed when a hydrogel-forming polymer (e.g. natural or synthetic polymer) is cross-linked (e.g. via physical interactions such as ionic, hydrophobic interaction or hydrogen bonding) to create a three-dimensional matrix structure which entraps water molecules to form a gel. For example, referring to FIG. 6B, a plurality of hydrogel beads comprising of cross-linked alginate is shown, wherein the cells at the centre of each bead are encapsulated by a three-dimensional matrix of cross-linked hydrogel polymer.

### Hydrogel-Forming Polymer

The hydrogel encapsulating or suspending the cells comprises one or more physically cross-linked hydrogel-forming polymers. As used herein, the term “hydrogel-forming polymer” refers to any polymer (or monomers which can subsequently form a polymer) which is capable of being cross-linked (e.g. cross-linked by physical interactions) to form a hydrogel. For example, an alginate hydrogel comprises sodium alginate as the hydrogel-forming polymer which can be ionically cross-linked by a polyvalent cations (such as Ca²⁺) to form a three-dimensional alginate matrix.

In the context of the present disclosure, the hydrogel-forming polymer is not toxic to ciliate cells, and allows sufficient diffusion of oxygen and nutrients to the ciliate cells encapsulated or suspended within the hydrogel to maintain cell viability. The hydrogel should also provide a surrounding that is resilient enough to withstand external abrasion and/or adverse forces (e.g. during storage) while remaining pliable enough to allow for the eventual release of the ciliate cells upon grazing by pest species (e.g. a slug) and/or contact with a suitable environment (e.g. rain). For example, the hydrogel comprises an outer surface which provides a protective barrier to mechanical stress, facilitates handling and/or maintains capsule hydration and/or is of suitable gelation strength to maintain a degree of structural integrity during storage and handling.

For example, the hydrogel-forming polymer is often biocompatible, water-soluble (i.e. hydrophilic), has pendant functional groups, and is cross-linked via physical cross-linking (e.g. ionically cross-linked) to form hydrogels where an interstitial aqueous liquid and ciliate cells may be encapsulated or suspended within. Functional groups of the hydrogel-forming polymer that facilitate the ionic cross-linking include for example, carboxyls, hydroxyls, primary or secondary amines, aldehydes, ketones, esters, and combinations thereof.

The hydrogels of the present invention may be made from a variety of hydrogel-forming polymers, including hydrophilic acrylics, peptides, dendrimers, star-polymers, aliphatic polymers, natural polymers, synthetic polymers, anionic polymers, cationic polymers, neutral polymers, and synthetic polymers, and any co-polymer thereof.

In one embodiment, the hydrogel-forming polymer may be made from a naturally occurring polymer, for example a polysaccharide.

In one embodiment, the hydrogel-forming polymer is a hydrophilic polymer. In one embodiment, the hydrogel comprises a physically cross-linked hydrophilic polymer.

Examples of hydrogel-forming polymers which can be used to form the hydrogels of the present invention include, but are not limited to, polylactic acid, polyglycolic acid, PLGA polymers, alginates and alginate salts/derivatives, collagen, fibrin, agarose, cellulose, gellan gum, starch, chitosan, chitin, carrageenan, or carboxymethylcellulose (CMC), gelatin, pectin, natural and synthetic polysaccharides, polyamino acids such as polypeptides e.g. poly(lysine), polyesters such as polyhydroxybutyrate and poly-epsilon.-caprolactone, polyanhydrides; polyphosphazines, poly(vinyl alcohols), poly(alkylene oxides) e.g. poly(ethylene oxides), poly(allylamines)(PAM), poly(acrylates), modified styrene polymers such as poly(4-aminomethylstyrene), pluronic polyols, polyoxamers, poly(uronic acids), poly(vinylpyrrolidone), polyacrylamide, poly(ethylene glycol dimethacrylate), poly(anhydride) or poly(vinylpyrrolidone), mixtures and copolymers of the above.

In one embodiment, the hydrogel-forming polymer is a polysaccharide, for example, the hydrogel may comprise a physically cross-linked polysaccharide. In some embodiments, the hydrogel-forming polymer is alginate or carboxymethylcellulose (CMC), or a mixture or co-polymer thereof. Other suitable polysaccharides used as hydrogel-forming polymers to form the hydrogel include the water-soluble salts of alginic, pectic and hyaluronan (hyaluronic acid), the water-soluble salts or esters of polyglucuronic acid, polymanuronic acid, polylygalacturonic acid and polyarabinic acid, and gum kappa-carrageenan. Other suitable polysaccharides include cellulose, gellan gum, starch, chitosan, and chitin. In one embodiment, the hydrogel-forming polymer is a polysaccharide selected from the group consisting of alginate, carboxymethylcellulose, cellulose, gellan gum, chitosan, and chitin, or mixtures or co-polymers thereof.

In some embodiments, the hydrogel-forming polymer is selected from one or more of alginate, collagen, fibrin, agarose, cellulose, gellan gum, starch, chitosan, chitin or carboxymethylcellulose (CMC), or a mixture or copolymer thereof.

Alternatively, in one embodiment, the hydrogel and/or hydrogel forming polymer does not comprise or consist of starch.

In one preferred embodiment, the hydrogel-forming polymer is alginate. The present inventors have identified that, in some embodiments, alginate hydrogels can encapsulate encysted ciliate cells which remain encysted and viable during storage, and can excyst to form theront cells once released from the hydrogel, and can infect pests such as molluscs. In one embodiment, the composition comprises an alginate hydrogel and a population of ciliate cells, wherein the ciliate cells are encapsulated or suspended within the alginate hydrogel.

As used herein, “alginate” is the general name given to alginic acid and its salts, and is composed of D-mannosyluronic (mannuronic—“M”) and L-gulopyranosyluronic (guluronic—“G”) acid residues. The ratio of mannuronic to guluronic acid residues is known as the M:G ratio. The 1,4-linked alpha.-1-guluronate (G) and beta.-d-mannuronate (M) are arranged in homopolymeric (GGG blocks and MMM blocks) or heteropolymeric block structures (MGM blocks). Various advantages are provided by using alginate as the hydrogel-forming polymer in the present invention, including good biocompatibility and low toxicity.

In some embodiments, the hydrogel-forming polymer is selected from the group consisting of ammonium, magnesium, potassium, sodium and other alkali metal salts of alginic acid (also referred to as an alginate salt).

In one preferred embodiment, the hydrogel-forming polymer is sodium alginate.

Sodium alginate is the sodium salt of alginic acid. Its empirical formula is (NaC₆H₇O₆)ₙ. Sodium alginate is a linear copolymer containing blocks of (1,4)-linked β-D-mannuronate (M) and a-L-gularonate (G) residues. The blocks are composed of consecutive G residues (GGGGGG), consecutive M residues (MMMMMM) and alternating M and G residues (GMGMGM). The amount, distribution and length of each block depends on the species, location and age of the seaweed from which the alginate is isolated. Other suitable alginates may also include potassium alginates, magnesium alginates and ammonium alginates.

In one embodiment, the hydrogel-forming polymer is sodium alginate (medium viscosity) purchased from Sigma Aldrich, catalogue number A2033.

In some embodiments, the hydrogel comprises about 0.1% w/v to about 20% w/v of the hydrogel-forming polymer. In some embodiments, the hydrogel comprises at least about 0.1%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v of the hydrogel-forming polymer. In other embodiments, the hydrogel comprises less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7,%. 0.6%, 0.5%, 0.4%, 0.3%, 0.2%, or 0.1% w/v of hydrogel-forming polymer. Combinations of these hydrogel-forming polymer concentrations to form various ranges are also possible, for example the hydrogel comprises about 0.1% w/v to about 15% w/v, about 0.5% w/v to about 10% w/v, about 1% w/v to about 5% w/v hydrogel-forming polymer. In some embodiments, the hydrogel comprises at least about 0.1%, 0.5%, 1%, 1.5%, 2%, 2.5%, 3%, 3.5%, 4%, 4.5%, or 5% w/v of the hydrogel-forming polymer. In other embodiments, the hydrogel comprises less than about 5%, 4.5%, 4%, 3.5%, 3%, 2.5%, 2%, 1.5%, 1%, 0.5%, or 0.1% w/v of hydrogel-forming polymer. In some embodiments, the hydrogel comprises about 1% to about 2% w/v of the hydrogel-forming polymer. In other embodiments, the hydrogel comprises about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%. 1.7%, 1.8%, 1.9% or 2% w/v of the hydrogel-forming polymer. In one embodiment, the hydrogel comprises about 1.5% of the hydrogel-forming polymer.

In some embodiments, the hydrogel-forming polymer has an average molecular weight (Mw) in the range of about 10,000 kg/mol to about 2,500,000 kg/mol. In some embodiments, the hydrogel-forming polymer has an average molecular weight (Mw) of at least about 10,000, 50,000, 100,000, 150,000, 200,000, 500,000, 700,000, 1,000,000, 1,200,000, 1,500,000, 2,000,000 or 2,500,000 kg/mol. In other embodiments, the hydrogel-forming polymer has an average molecular weight (Mw) of less than about 2,500,000, 2,000,000, 1,500,000, 1,000,000, 700,000, 500,000, 200,000, 150,000, 100,000, 50,000, or 10,000 kg/mol. Combinations of these molecular weights to form various ranges are also possible, for example the hydrogel-forming polymer has an average molecular weight (Mw) of about 200,000 to about 1,500,000 kg/mol, or about 500,000 kg/mol to about 700,000 kg/mol. It will be appreciated that the molecular weight of the hydrogel-forming polymer may vary depending on the type used to prepare the hydrogels. For example, different grades of alginate can be used which would vary in molecular weight.

In some embodiments, the hydrogel-forming polymer is sodium alginate. The sodium alginate may have a molecular weight in the range of about 10,000 kg/mol to about 600,000 kg/mol.

In some embodiments, the alginate has an M:G ratio in the range of about 0.2 to about 3.5. Without wishing to be bound by theory, it is believed that mainly guluronic acid residues are responsible for the cross-linking of the alginate monomers with ionic cross-linking agents, such as Ca²⁺ ions. As such, the lower alginate M:G ratio is, the stronger the resulting alginate hydrogel is once cross-linked.

In some embodiments, the hydrogel comprises about 0.1% w/v to about 5% w/v alginate. In some embodiment, the hydrogel comprises at least about 0.1%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, or 5% w/v alginate. In other embodiments, the hydrogel comprises less than about 5%, 4.5%, 4%. 3.5%, 3%, 2.5%, 2%, 1.5%, 1.4%, 1.3%, 1.2%, 1.1%, 1%, 0.9%, 0.8%, 0.7,%. 0.6%, 0.5%, 0.4%, 0.3%, 0.2%, or 0.1% w/v alginate. Combinations of these alginate concentrations to form various ranges are also possible, for example the hydrogel comprises about 1% w/v to about 2% w/v, or about 1.5% w/v to about 3% w/v alginate. In some embodiments, the hydrogel comprises about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%. 1.7%, 1.8%, 1.9% or 2% w/v alginate. In some embodiments, the hydrogel comprises about 0.1% w/v to about 20% w/v alginate. In some embodiments, the hydrogel comprises at least about 0.1%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v alginate. In other embodiments, the hydrogel comprises less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7,%. 0.6%, 0.5% 0.4%, 0.3%, 0.2%, or 0.1% w/v alginate. Combinations of these concentrations to form various ranges are also possible, for example the hydrogel comprises about 0.1% w/v to about 15% w/v, about 0.5% w/v to about 10% w/v, about 1% w/v to about 5% w/v alginate.

In another embodiment, the hydrogel-forming polymer is carboxymethylcellulose (CMC). The inventors have identified that, in some embodiments, CMC hydrogels can suspend trophont and encysted ciliate cells which remain stable and viable. In one embodiment, the composition comprises a carboxymethylcellulose (CMC) hydrogel and a population of ciliate cells, wherein the ciliate cells are encapsulated or suspended within the CMC hydrogel. In some embodiments, the hydrogel comprises about 0.1% w/v to about 5% w/v carboxymethylcellulose. In some embodiment, the hydrogel comprises at least about 0.1%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, or 5% w/v carboxymethylcellulose. In other embodiments, the hydrogel comprises less than about 5%, 4.5%, 4%. 3.5%, 3%, 2.5%, 2%, 1.5%, 1.4%, 1.3%, 1.2%, 1.1%, 1%, 0.9%, 0.8%, 0.7,%. 0.6%, 0.5%, 0.4%, 0.3%, 0.2%, or 0.1% w/v carboxymethylcellulose. Combinations of these carboxymethylcellulose concentrations to form various ranges are also possible, for example the hydrogel comprises about 0.5% w/v to about 4% w/v, about 1% w/v to about 2% w/v, or about 1.5% w/v to about 3% w/v carboxymethylcellulose. In some embodiments, the hydrogel comprises about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%. 1.7%, 1.8%, 1.9% or 2% w/v carboxymethylcellulose. In one embodiment, the hydrogel comprises about 1.5% w/v carboxymethylcellulose. In some embodiments, the hydrogel comprises about 0.1% w/v to about 20% w/v carboxymethylcellulose. In some embodiments, the hydrogel comprises at least about 0.1%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v carboxymethylcellulose. In other embodiments, the hydrogel comprises less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7,%. 0.6%, 0.5%, 0.4%, 0.3%, 0.2%, or 0.1% w/v carboxymethylcellulose. Combinations of these concentrations to form various ranges are also possible, for example the hydrogel comprises about 0.1% w/v to about 15% w/v, about 0.5% w/v to about 10% w/v, about 1% w/v to about 5% w/v carboxymethylcellulose.

### Cross-Linked Hydrogel Forming Polymer

The hydrogel-forming polymer is physically cross-linked to form the hydrogel. As used herein, the term “cross-link, “cross-linked” or “cross-linking” refers to the formation of interactions within or between hydrogel-forming polymers which result in the formation of a three-dimensional matrix. i.e. a hydrogel. For example, sodium alginate may be cross-linked by calcium cations (Ca²⁺) to form an alginate hydrogel.

The term “physically cross-linked” refers to a type of cross-linking that is reversible in nature (i.e. not permanent) as opposed to chemically cross-linked hydrogels (i.e. permanent). Examples of physical cross-linking includes molecular entanglement of the hydrogel-forming polymer, ionic interactions, hydrogen bonding and hydrophobic interaction.

In one embodiment, the hydrogel-forming polymer is ionically-cross linked (e.g. linked by ionic interactions (i.e. an electrostatic attraction between oppositely charged ions). For example, the ionic-cross linking may be a charge interaction between the hydrogel-forming polymer and an oppositely charged molecule as the linker. This charged small molecule may be a polyvalent cation or anion. The oppositely charged molecule may also be a polymer. The ionic-cross linking may also be between two hydrogel forming polymers of the opposite charge. Various other suitable types of cross-linking are described in Parhi et al. (2017).

In some embodiments, depending on the nature of the hydrogel-forming polymer, the hydrogel-forming polymer is cross-linked by a polyvalent cation. The term “polyvalent cation” refers to a cation with a positive charge equal or greater than +2 (e.g. Ca²⁺, Fe³⁺).

In some embodiments, the concentration of the polyvalent cation in the hydrogel is about 20 mM to about 500 mM. In some embodiments, the concentration of the polyvalent cation in the hydrogel is at least about 20, 30, 40, 50, 60, 70, 80, 90, 100, 150, 200, 250, 300, 350, 400, 450, or 500 mM. In other embodiments, the concentration of the polyvalent cation in the hydrogel is less than about 500, 450, 400, 350, 300, 250, 200, 150, 100, 90, 80, 70, 60, 50, 40, 30, 20, or 10 mM. Combinations of these concentrations to form various ranges are also possible, for example the concentration of the polyvalent cation in the hydrogel is about 10 mM to about 300 mM, 20 mM to about 200 mM, or about 40 mM to about 100 mM. In one embodiment, the concentration of the polyvalent cation cations in the hydrogel is about 40 mM to about 60 mM, for example about 50 mM.

In some embodiments, the concentration of the polyvalent cation in the hydrogel is about 0.05% to about 1.5% w/v. In some embodiments, the concentration of the polyvalent cation in the hydrogel is at least about 0.05, 0.1, 0.2, 0.3, 0.4, 0.5, 0.6, 0.7, 0.8, 0.9, 1.0, 1.1, 1.2, 1.3, 1.4, or 1.5% w/v. In other embodiments, the concentration of the polyvalent cation in the hydrogel is about 1.5, 1.4, 1.3, 1.2, 1.1, 1.0, 0.9, 0.8, 0.7, 0.6, 0.5, 0.4, 0.3, 0.2, 0.1, or 0.05% w/v. Combinations of these values to form various ranges are also possible, for example the concentration of the polyvalent cation in the hydrogel is about 0.1% w/v to about 1.3% w/v, about 0.2% w/v to about 1.1% w/v, or about 0.3% w/v to about 0.7% w/v, for example about 0.5% w/v. In some embodiments, the concentration of the polyvalent cation in the hydrogel can also be controlled by altering the exposure time of the hydrogel-forming polymer in the cross-linker solution, hydrogel bead size, and/or concentration of the cross-linker solution used to prepare the hydrogel.

In some embodiments, the hydrogel is ionically cross-linked by divalent cations or trivalent cations, or mixtures thereof.

In some embodiments, the polyvalent cation is a divalent cation. As used herein, the term “divalent cation” is intended to mean a positively charged element, atom or molecule having a valence of +2. The divalent cation may be selected from one or more of Ca²⁺, Mg²⁺, Sr²⁺, Ba²⁺, Zn²⁺, or Be²⁺, and salt forms of these cations (e.g. CaCl₂). In one preferred embodiment, the hydrogel-forming polymer is cross-linked by Ca²⁺.

In other embodiments, the polyvalent cation is a trivalent cation. As used herein, the term “trivalent cation” is intended to mean a positively charged element, atom, or molecule having a valence of +3. The trivalent cation may be selected from one or more of Fe³⁺, Al³⁺, or Mn³⁺, and salt forms of these cations (e.g. FePO₄, FeCl₃, and AlCl₃). In one embodiment, the hydrogel-forming polymer is cross-linked by Fe³⁺.

In some embodiments, the cross-linking cation is a mixture of both divalent and trivalent cations, both of which may be selected from the cations as described herein.

In one embodiment, the hydrogel-forming polymer is sodium alginate and the cross-linking cations are Ca²⁺. The reaction between Ca²⁺ ions and sodium alginate is: 2NaAlg+Ca²⁺⇄CaAlg₂+2Na⁺. That is, the Ca²⁺ cross-links two alginate molecules to form the hydrogel by displacing the sodium from the sodium alginate hydrogel-forming polymer. Therefore, it will be appreciated that when sodium alginate is the hydrogel-forming polymer, the sodium cations are not a component of the hydrogel. Therefore, while sodium alginate may be used as the hydrogel-forming polymer, it is the alginate which gets cross-linked to form the alginate hydrogel.

Sources for the Ca²⁺ ions used in the formation of alginate gels include, for example, calcium carbonate, calcium sulfate, calcium chloride, calcium phosphate, calcium tartrate, calcium nitrate, and calcium hydroxide. In one preferred embodiment, the source of the Ca²⁺ ions is calcium chloride (CaCl₂).

The present inventors have surprisingly identified that, in some embodiments, the density and or/nature of the cross-linking at the surface of the hydrogel stimulates the migration of trophont ciliate cells to the centre of the hydrogel which subsequently undergo encystment. Without wishing to be bound by theory, it is believed that the migration of trophont cells to the centre of the hydrogel is caused by the trophont cells aversion to the high cross-linking density and/or cross-linker cation at the surface of the hydrogel.

The water content of the hydrogel can be varied within wide ranges. In some embodiments, the hydrogel comprises about 80% w/v to about 99.9% w/v water. In some embodiments, the hydrogel comprises at least about 80%, 85%, 90%, 95%, 95.5%, 96%, 96.5%, 97%, 97.5%, 98%, 98.5%, 99%, or 99.5% w/v water. In other embodiments, the hydrogel comprises less than about 99.5%, 99%, 98.5%, 98%, 97.5%, 97%, 96.5%, 96%, 95.5%, 95%, 90%, 85%, or 80% w/v water. Combinations of these values to form various ranges are also possible, for example the hydrogel comprises about 90% to about 99% w/v water, or about 95% to about 98.5% w/v water. In one embodiment, the hydrogel comprises about 98% w/v water.

In some embodiments, the hydrogel may comprise about 0.1% w/v to about 20% w/v hydrogel-forming polymer, about 0.05% w/v to about 1.5% w/v polyvalent cations, and about 80% w/v to about 99.9% w/v water. For example, the hydrogel may comprise about 1.5% w/v hydrogel-forming polymer, 0.5% w/v polyvalent cations, and about 98% w/v water. In one embodiment, the hydrogel may comprise about 1.5% w/v alginate, 0.5% w/v Ca²⁺ cations, and about 98% w/v water.

In some embodiments, the hydrogel may be porous or non-porous.

The hydrogel may further comprise one or more additional components.

In some embodiments, the hydrogel further comprises magnesium sulfate. The present inventors have identified that, in some embodiments, sulfate anions, such as magnesium sulfate, as an additional additive may also trigger the encystment of trophont ciliate cells within the hydrogel. In some embodiments, the concentration of magnesium sulfate in the hydrogel is about 10 μM to about 100 μM. In some embodiments, the concentration of magnesium sulfate in the hydrogel is at least about 10, 20, 30, 40, 50, 60, 70, 80, 90, or 100 μM. In other embodiments, the concentration of magnesium sulfate in the hydrogel is less than about 100, 90, 80, 70, 60, 50, 40, 30, 20, or 10 μM. Combinations of these magnesium sulfate concentrations to form various ranges are also possible, for example the concentration of magnesium sulfate in the hydrogel is about 10 μM to about 90 μM, about 20 μM to about 80 μM, or about 50 μM to about 70 μM. In one embodiment, the concentration of magnesium sulfate in the hydrogel is about 60 μM to about 60 μM, for example about 62.5 μM.

In some embodiments, the concentration of magnesium sulfate in the hydrogel is about 0.01% w/v to about 0.15% w/v. In one embodiment, the concentration of magnesium sulfate in the hydrogel is about 0.075% w/v.

The hydrogel may further comprise additional components, such as preservatives including parabens, benzoates, sorbic acid, citrates or parabens, humectants such as glycerol or propylene glycol, antioxidants such as butylhydroxytoluene or butylhydroxyanisole, tocopherol, ascorbic acid, flavourings or other formulation auxiliaries.

In one embodiment, the hydrogel may further comprise optional additional components, such as an attractant or feeding stimulant. As used herein, the term “attractant” refers to an agent which assists in attracting one or more pest species to consume the hydrogel. As used herein, the term “feeding stimulant” refers to an agent that encourages one or more pest species to remain consuming the hydrogel for a period of time to allow for the rupture and release of ciliate cells encapsulated or suspended within and be exposed to the ciliate cells. However, it will be appreciated that the hydrogels can be consumed and ruptured by a pest species without the use of attractants and/or feeding stimulants. The attractant may include a pheromone or a nutrient source. In some embodiments, the attractant may be selected from one or more of a starch, carbohydrate, protein, amino acid, plant extracts (e.g. any one of essential oils, saps, resins, chlorophyll and other crude extracts of a plant that a slug may detect as a food source) or a pheromone.

In one embodiment, the hydrogel comprises a nutrient source. The nutrient source may be a molasses or a sugar. In some embodiments, the nutrient source may be selected from the group consisting of starch, sugar, semolina, couscous or combination thereof. The nutrient source may also be a carbohydrate or a plant product.

In one embodiment, the hydrogel comprises a feeding stimulant. Other attractants may also be used. The attractant or feeding stimulant may be provided as an outer coating on the hydrogel, for example as an outer coating on the plurality of hydrogel beads. In some embodiments, the feeding stimulant may be selected from one or more of a starch, carbohydrate, protein, amino acid, plant extracts (e.g. any one of essential oils, saps, resins, chlorophyll and other crude extracts of a plant that a slug may detect as a food source)

In some embodiments, the hydrogel may comprise a single species of ciliate, multiple species of ciliate, or one or more species of ciliates with other organisms, such as pathogenic bacteria, fungal spores, or pathogenic nematodes. Accordingly, in some embodiments, the hydrogel may further comprise one or more other additional components such as one or more of a bait, pesticide, biocontrol agent, or other organisms such as pathogenic bacteria, fungal spores or pathogenic nematodes. In one embodiment, the hydrogel further comprises pathogenic bacteria or fungal spores. The pathogenic bacteria or fungal spores may be encapsulated or suspended within the hydrogel with the ciliate cells, or may be inside the ciliate cells prior to encapsulation or suspension within the hydrogel. Without wishing to be bound by theory, it is believed that as some bacteria and fungi are pathogenic to pest species, such as slugs and snails, the presence of both ciliate cells and bacteria or fungal spores may result in a higher killing effect. For example, ciliate cells that have ingested bacteria and/or fungi may be released from the hydrogel and subsequently enter or be ingested by a pest species (e.g. a slug). Once inside the pest species, the ciliate cells may release the bacteria and/or fungi which also has an adverse effect on the pest species, thus resulting in a higher killing effect.

Alternatively, in some embodiments, the hydrogel does not encapsulate or suspend a fungi, such as an entomopathogenic fungi, e.g. a fungi selected from Metarhizium roberstsii, Metarhizium anisopliae, and Beauveria bassiana. In some embodiments, the hydrogel does not encapsulate or suspend one or more of a spore, a microsclerotia, hyphae, a mycelium, or a conidia. In some embodiments, the hydrogel does not encapsulate or suspend a bacteria, for example Bacillus thuringiensis, Bacillus sphaericus, and Bacillus popillae. In some embodiments, the hydrogel does not encapsulate or suspend a virus, for example Autographa california nuclear polyhedrosis virus or Heliothis spp. virus.

In another embodiment, the hydrogel further comprises iron(III) phosphate (FePO₄).

Encapsulation/Suspension of Cells within the Hydrogel

The hydrogel in the composition encapsulates or suspends ciliate cells. The ciliate cells may be trophont ciliate cells encysted ciliate cells, and/or theront ciliate cells. In one embodiment, the ciliate cells are encysted ciliate cells. The present inventors have identified that, in some embodiments, depending on the hydrogel properties, trophont ciliate cells can be suspended or encapsulated within the hydrogel and either undergo encystment within the hydrogel to form encysted ciliate cells or remain suspended within the hydrogel as trophont ciliate cells. The present inventors have also identified that, in some embodiments, the hydrogel compositions can encapsulate pre-formed encysted ciliate cells. Regardless of the stage of ciliate cell, when encapsulated or suspended within the hydrogel, the ciliate cells can be stored and remain viable, in contrast to conventional methods of growing and harvesting trophont and encysted ciliate cells which are delicate and spontaneously encyst and/or excyst.

In one embodiment, a population of ciliate cells are suspended within the hydrogel. In another embodiment, a population of ciliate cells are encapsulated within the hydrogel. Depending on the life stage of the ciliate cells being encapsulated within the hydrogel beads, the ciliate cells may be either evenly dispersed throughout the hydrogel beads, or alternatively may reside at the centre of the hydrogel.

The present inventors have identified that, in some embodiments, high cross-linking density within the hydrogel can trigger cell migration and encystment. For example, trophont ciliate cells encapsulated within hydrogel beads (e.g. within alginate hydrogel beads) which are trapped within a surface of high-density cross-linked hydrogel migrate to the centre of the hydrogel bead and subsequently encyst into encysted ciliate cells and remained encysted during storage. In contrast, pre-formed encysted ciliate cells did not migrate and rather remained evenly dispersed throughout the encapsulating hydrogel.

The inventors also identified that, in some embodiments, trophont ciliate cells suspended within a hydrogel (i.e. not encapsulated) did not undergo encystment, and remained as trophont cells. Without wishing to be bound by theory, the inventors believe this is due to the lack of a high density of cross-linked gel around the cells as opposed to when the cells are encapsulated within a bead. In this embodiment, the trophont cells do not migrate together to any one particular point in the hydrogel and rather are evenly distributed throughout the hydrogel, and thus encystment is not triggered. This demonstrates that a high density of cross-linking within the hydrogel triggers encystment of encapsulated trophont ciliate cells not suspended cells. Surprisingly, in some embodiments, pre-formed encysted ciliate cells that were either suspended or encapsulated in a hydrogel did not migrate within the hydrogel and rather remained dispersed throughout the hydrogel, and also remained encysted ciliate cells during storage. As such, the present inventors have demonstrated that the developmental stage of ciliate cells encapsulated or suspended within a hydrogel can be altered depending on the properties of the hydrogel, whilst still maintaining good cell viability and stability.

It will be appreciated that the compositions of the present invention comprise a hydrogel as defined herein and a population of ciliate cells (e.g. encysted ciliate cells, trophont ciliate cells and/or theront ciliate cells). In some embodiments, the composition of the present invention comprises at least about 0.5, 1, 2, 5, 10, 15, 20, 25, 30, 35 or 40% w/v ciliate cells. In some embodiments, the composition comprises less than about 40, 35, 30, 25, 20, 15, 10, 5, 2, 1 or 0.5% w/v ciliate cells. Combinations of these % w/v values are also possible, for example the composition may comprise about 0.5% w/v to about 40% w/v ciliate cells.

In some embodiments, the composition comprises about 0.5% w/v to about 40% w/v ciliate cells, about 0.1% w/v to about 15% w/v hydrogel forming polymer, about 80% to about 99% w/v water, and about 0.05% to about 1.5% w/v of polyvalent cation.

Other % w/v values described herein in relation to the hydrogel also apply to the composition.

In some embodiments, the hydrogel comprises a plurality of hydrogel beads, wherein one or more of the hydrogel beads encapsulates one or more of the ciliate cells. The hydrogel beads may be spherical or slightly irregular in shape (e.g. a teardrop morphology). The beads may be discrete beads with discrete centres comprising the ciliate cells (see FIG. 6B).

In some embodiments, the hydrogel comprises a plurality of hydrogel beads. In some embodiments, the average size of the beads is about 100 μm (0.1 mm) to about 100 mm. In some embodiments, the hydrogel beads have an average size of at least about 0.1, 0.2, 0.3, 0.4, 0.5, 0.6, 0.7, 0.8, 0.9, 1, 2, 3, 4, 5, 6, 7, 8, 9, 10, 15, 20, 30, 50, 70, or 100 mm. In other embodiments, the hydrogel beads have an average size of less than about 100, 70, 50, 30, 20, 15, 10, 9, 8, 7, 6, 5, 4, 3, 2, 1, 0.9, 0.8, 0.7, 0.6, 0.5, 0.4, 0.3, 0.2, or 0.1 mm. Combinations of these average hydrogel bead sizes to form various ranges are also possible, for example the hydrogel beads have an average size of about 0.5 mm to about 50 mm, about 1 mm to about 40 mm, or about 5 mm to about 30 mm. In one embodiment, the hydrogel beads have an average size of about 0.1 mm to about 5 mm, for example about 1 mm to 4 mm.

The average size of the hydrogel beads can be measured using any suitable means, for example an optical microscope or ruler. The size is taken to be the largest cross-sectional distance across a single bead, for example the diameter if the bead is spherical.

In other embodiments, the hydrogel comprises a plurality of hydrogel beads, wherein the average size of the beads is about 1 mm to about 100 mm. In some embodiments, the hydrogel beads have an average size of at least about 1, 2, 3, 4, 5, 6, 7, 8, 9, 10, 15, 20, 30, 50, 70, or 100 mm. In other embodiments, the hydrogel beads have an average size of less than about 100, 70, 50, 30, 20, 15, 10, 9, 8, 7, 6, 5, 4, 3, 2, or 1 mm. Combinations of these average hydrogel bead sizes to form various ranges are also possible, for example the hydrogel beads have an average size of about 1 mm to about 50 mm, about 5 mm to about 40 mm, or about 10 mm to about 30 mm. In one embodiment, the hydrogel beads have an average size of about 1 mm to about 5 mm, for example about 3 mm to 4 mm.

In some embodiments, the hydrogel comprises a plurality of hydrogel beads, wherein the average number of ciliate cells encapsulated in the one or more hydrogel beads is about 100 to about 10,000 ciliate cells per bead. In some embodiments, the average number of ciliate cells encapsulated in the one or more hydrogel beads is at least about 100, 200, 300, 400, 500, 600, 700, 800, 900, 1000, 2000, 3000, 4000, 5000, 6000, 7000, 8000, 9000 or 10,000 ciliate cells per bead. In other embodiments, the average number of ciliate cells encapsulated in the one or more hydrogel beads is less than about 10,000, 9000, 8000, 7000, 6000, 5000, 4000, 3000, 2000, 1000, 900, 800, 700, 600, 500, 400, 300, 200, or 100 ciliate cells per bead. Combinations of these average cells per bead values to form various ranges are also possible, for example the average number of ciliate cells encapsulated in the one or more hydrogel beads is about 500 to 2000 ciliate cells per bead, or about 700 to 1500 ciliate cells per bead. In one embodiment, the average number of ciliate cells encapsulated in the one or more hydrogel beads is about 1000 ciliate cells per bead.

In some embodiments, the hydrogel comprises a plurality of hydrogel beads, wherein one or more of the hydrogel beads encapsulates one or more of the ciliate cells. In some embodiments, at least 50% of the plurality of hydrogel beads in the composition encapsulates one or more ciliate cells For example, at least 50%, 60%, 70%, 80%, or 90% of the hydrogel beads encapsulates one or more ciliate cells. In some embodiments, at least 90%, 91%, 92%, 93%, 94%, 95%, 96%, 97%, 98%, 99% or 99.5% of the plurality hydrogel beads encapsulates one or more of the ciliate cells.

In some embodiments, the hydrogel comprises a plurality of hydrogel beads, wherein the hydrogel beads comprise a hydrogel core and one or more outer hydrogel shells encapsulating the core, wherein the core comprises one or more ciliate cells. It will be appreciated that this morphology is a core/shell structure. The hydrogel core and shell may each comprise a physically cross-linked hydrogel forming polymer as described herein. The outer shell provides a surrounding that is resilient to withstand external abrasion and/or adverse forces (e.g. during storage) while remaining pliable enough to allow for the eventual release of the ciliate cells from the core upon grazing by the slugs and/or contact with a suitable environment (e.g. rain). Multiple outer shells may be layered onto the hydrogel cores, for example to include attractants that are required to not be in direct contact with the ciliate cells within the core.

In one embodiment, the plurality of hydrogel beads comprise a cross-linked carboxymethylcellulose core and one or more cross-linked alginate outer shells (e.g. a CMC-alginate core-shell hydrogel particle). The one or more outer shells may comprise an attractant as described herein.

The core/shell hydrogel beads may have an average core size about 10 μm (0.01 mm) to about 100 mm. In some embodiments, the average core size is at least about 0.1, 0.2, 0.3, 0.4, 0.5, 0.6, 0.7, 0.8, 0.9, 1, 2, 3, 4, 5, 6, 7, 8, 9, 10, 15, 20, 30, 50, 70, or 100 mm. In other embodiments, the average core size is less than 100, 70, 50, 30, 20, 15, 10, 9, 8, 7, 6, 5, 4, 3, 2, 1, 0.9, 0.8, 0.7, 0.6, 0.5, 0.4, 0.3, 0.2, or 0.1 mm. Combinations of these average sizes to form various ranges are also possible, for example average core size is about 0.5 mm to about 50 mm, about 1 mm to about 40 mm, or about 5 mm to about 30 mm. In one embodiment, the average core size of about 0.1 mm to about 5 mm, for example about 1 mm to 4 mm.

The core/shell hydrogel beads may have an average outer shell width of about 1 μm (0.001 mm) to about 1 mm. In some embodiments, the average outer shell width may be at least about 1, 2, 5, 10, 15, 20, 50, 100, 200, 500, 800 or 1000 μm. In some embodiments, the average outer shell width may be less than about 1000, 800, 500, 200, 100, 50, 20, 15, 10, 5, 2 or 1 μm. Combinations of these average outer shell widths are also possible, for example about 10 μm to about 500 μm. The core and shell dimensions may be measured using an optical microscope.

It will be appreciated that the compositions of the present invention comprise a hydrogel as defined above and a population of ciliate cells. In some embodiments, the composition of the present invention comprises about 0.5% w/v to about 40% w/v ciliate cells and about 60% w/v to about 99.5% w/v of the hydrogel as defined above. For example, the composition may comprise about 10% w/v ciliate cells and up to about 90% w/v of the hydrogel. In another example. The composition may comprise about 5% w/v ciliate cells and up to about 95% w/v of the hydrogel.

In some embodiments, the hydrogel may be dehydrated to form one or more granules. It will be appreciated that the dehydrated hydrogel can be rehydrated upon contact with a suitable aqueous environment, such as water (e.g. rain or a sprinkler following application to an environment). The hydrogel may be dried using any conventional means, such as room temperature airflow, mild heat and/or vacuum. The ciliate cells encapsulated or suspended within the hydrogel remain viable even after dehydration.

In some embodiments, optional additional components can be added to the composition that are not part of the hydrogel. For example, the hydrogel encapsulating/suspending the ciliate cells may be suspended in a small volume of water (e.g. less than 5% w/v of the overall composition). Alternatively, the hydrogel may be suspended or mixed with an agriculturally or horticulturally acceptable carrier. As used herein, an “acceptable carrier” and/or an “agriculturally suitable carrier” and/or an “horticulturally acceptable carrier” is any carrier which can facilitate the transport, application or persistence of the compositions, ciliate cells and/or isolated strains to an area affected or likely to be affected by a pest species (such as an invertebrate), and which is otherwise suitable for agricultural and/or horticultural use, including but not limited to home garden and vegetation uses. Any such suitable acceptable carrier can be used, including but not limited to seeds, seed coats, granular carriers, liquid slurry carriers, and liquid suspension carriers. Suitable carriers are defined below.

In another embodiment, the composition may comprise other optional additional components, such as preservatives including parabens, benzoates, sorbic acid, citrates or parabens, humectants such as glycerol or propylene glycol, antioxidants such as butylhydroxytoluene or butylhydroxyanisole, tocopherol, ascorbic acid, flavourings or other formulation auxiliaries.

In yet another embodiment, the composition may further comprise one or more further optional components such as an attractant, bait, pesticide, biocontrol agent, or one or more other organisms, such as pathogenic bacteria, fungal spores or pathogenic nematodes. For example, additional biocontrol agents may be added to the composition separate to the hydrogel. In another embodiment, the composition may further comprise one or more metallic salts, for example metallic phosphates and metallic sulfates. In some embodiments, the composition may further comprise iron(III) phosphate (FePO₄), iron (II) phosphate (Fe₃(PO₄)₂) and copper (II) sulfate (CuSO₄). The embodiments described above in relation to the optional additional components of the hydrogel also apply in relation to the optional additional components of the composition.

In another embodiment, the present disclosure also provides a composition for control of pest species comprising an effective amount of one or more Tetrahymena ciliate cells. In some embodiments, the ciliate cells are of the T. rostrata, T. hegewischi, T. hyperangularis, T. malaccensis, T. patula, T. pigmentosa, T. pyriformis, T. thermophila, T. vorax, T. geleii, T. corlissi, T. empidokyrea, T. rotunda, or T. limacis species. Other species from the Tetrahymena genus are also envisaged. The composition may comprise hydrogels which encapsulate and/or suspend the ciliate cells as described herein. Alternatively, the composition may comprise a suitable agricultural or horticultural carrier as described herein, which carries the ciliate cells. As used herein, the term “effective amount” refers to a quantity of ciliate cells, a hydrogel encapsulating or suspending the ciliate cells, and/or a composition comprising the ciliate cells sufficient to control, kill, inhibit and/or reduce the number, emergence, or growth of a pathogen, pest, or insect, for example gastropods (e.g. slugs).

### Methods of Encapsulating or Suspending Ciliate Cells in Hydrogel

To encapsulate or suspend a population of ciliate cells within a hydrogel, one method comprises:

a) adding a suspension of ciliate cells to a hydrogel-forming polymer solution to form a hydrogel,

wherein the ciliate cells are encapsulated or suspended within the hydrogel.

In one embodiment, the hydrogel-forming polymer physically cross-links to form a hydrogel (for example via H-bonding or hydrophobic interaction between moieties located within the hydrogel-forming polymer e.g. where the hydrogel-forming polymer is a copolymer such as a poloxamer).

In some embodiments, the hydrogel-forming polymer is physically cross-linked (e.g. via ionic interactions). Thus, in one embodiment, step a) further comprises adding a suspension of ciliate cells to a hydrogel-forming polymer solution and an ionic cross-linker solution.

In some embodiments, adding the suspension of ciliate cells and the hydrogel-forming polymer solution to the ionic cross-linker solution forms the hydrogel and the ciliate cells are encapsulated or suspended within the hydrogel in situ as the hydrogel forms. In some embodiments, the ciliate cells migrate to the centre of the hydrogel. In other embodiments, the ciliate cells are evenly distributed throughout the hydrogel.

In one embodiment, the suspension of ciliate cells is a suspension of trophont ciliate cells. Alternatively, the suspension of ciliate cells is a suspension of pre-formed encysted ciliate cells.

In one embodiment, the suspension of ciliate cells is a suspension of trophont ciliate cells, wherein the trophont ciliate cells are encapsulated by the hydrogel and undergo encystment within the hydrogel to form one or more encysted ciliate cells.

The present inventors have found that, in some embodiments, trophont ciliate cells may be initially dispersed within the hydrogel-forming polymer solution, however during the cross-linking and formation of the hydrogel, the trophont ciliate cells migrate to the centre of the hydrogel and undergo encystment. As described above, it is believed that, in some embodiments, the migration of trophont cells to the centre of the hydrogel is caused by the trophont cells aversion to the high concentration of cross-linking at the surface of the hydrogel. As there is a decreasing gradient in cross-linker from surface of the hydrogel to its centre, the reduced availability of the cross-linker in the centre of the hydrogel would favour migration and encystment. In addition, the migration of the cells to the centre of the hydrogel results in a high cell density environment and the crowding and aggregation of the trophont ciliate cells at the centre of the hydrogel triggers the encystment of the trophont ciliate cells into encysted ciliate cells.

The inventors further identified that, in some embodiments, pre-encysted ciliate cells did not undergo such migration when encapsulated within a hydrogel yet still remained stable and viable, surprisingly highlighting that the density and/or nature of the cross-linker induced cell migration only in trophont ciliate cells.

In some embodiments, the density of the ciliate cells in the suspension of ciliate cells is from about 1×10² cells/mL to about 1×10¹⁰ cells/mL. For example, the density of the ciliate cells in the suspension may be about 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, or 1×10¹⁰ cells/mL. In one embodiment, the density of the ciliate cells in the suspension of ciliate cells is about 1×10⁵ cells/mL.

It will be appreciated that the embodiments provided above for the hydrogel-forming polymer with regard to the hydrogel also apply to the embodiments for the hydrogel-forming polymer solution.

In some embodiments, the concentration of the hydrogel-forming polymer in the hydrogel-forming polymer solution is about 0.1% w/v to about 20% w/v. In some embodiments, the concentration of the hydrogel-forming polymer in the hydrogel-forming polymer solution is at least about 0.1%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v. In other embodiments, the concentration of the hydrogel-forming polymer in the hydrogel-forming polymer solution is less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7%, 0.6%, 0.5%, 0.4%, 0.3%, 0.2%, or 0.1% w/v. Combinations of these hydrogel-forming polymer concentrations to form various ranges are also possible, for example the concentration of the hydrogel-forming polymer in the hydrogel-forming polymer solution is about 0.5% w/v to about 15% w/v, about 1% w/v to about 2% w/v, or about 1.5% w/v to about 3% w/v. In some embodiments, the concentration of the hydrogel-forming polymer in the hydrogel-forming polymer solution is about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9% or 2% w/v. In one embodiment, the concentration of the hydrogel-forming polymer in the hydrogel-forming polymer solution is about 1.5% w/v.

In some embodiments the vol:vol ratio of the suspension of ciliate cells to the hydrogel-forming polymer solution is about 1:10 to about 10:1. In some embodiments, the vol:vol ratio of the suspension of ciliate cells to the hydrogel-forming polymer solution is at least about 1:10, 1:8, 1:4, 1:2, 1:1, 2:1, 4:1, 6:1, 8:1, or 10:1. In some embodiments, the vol:vol ratio of the suspension of ciliate cells to the hydrogel-forming polymer solution is less than about 10:1, 8:1, 6:1, 4:1, 2:1, 1:1, 1:2, 1:4, 1:6, 1:8, or 1:10. Combinations of these vol:vol ratios to form various ranges are also possible, for example the vol:vol ratio of the suspension of ciliate cells to the hydrogel-forming polymer solution is about 1:8 to about 8:1, about 1:4 to about 4:1, about 1:10 to about 1:1, or 1:8 to about 1:2. In one embodiment, the vol:vol ratio is about 1:4.

In some embodiments, the ionic cross-linker solution comprises polyvalent cations. It will be appreciated that the embodiments provided above for the polyvalent cations with regard to the physically cross-linked hydrogel also apply to the embodiments for the polyvalent cations in the cross-linker cation solution.

In some embodiments, the concentration of the polyvalent cations in cross-linker solution is about 20 mM to about 500 mM. In some embodiments, the concentration of the polyvalent cations in cross-linker solution is at least about 20, 30, 40, 50, 60, 80, 90, 100, 150, 200, 250, 300, 350, 400, 450, or 500 mM. In other embodiments, the concentration of the polyvalent cations in cross-linker solution is less than about 500, 450, 400, 350, 300, 250, 200, 150, 100, 90, 80, 70, 60, 50, 40, 30, 20, or 10 mM. Combinations of these concentrations to form various ranges are also possible, for example the polyvalent cations in cross-linker solution is 10 mM to about 300 mM, 20 mM to about 200 mM, or about 40 mM to about 100 mM. In one embodiment, the concentration of the polyvalent cation cations in the hydrogel is about 40 mM to about 60 mM, for example about 50 mM.

In some embodiments, the concentration of the polyvalent cations in the cross-linker cation solution is about 0.05% and about 1.5% w/v. In some embodiments, the concentration of the polyvalent cations in cross-linker solution is at least about 0.05, 0.1, 0.2, 0.3, 0.4, 0.5, 0.6, 0.7, 0.8, 0.9, 1.0, 1.1, 1.2, 1.3, 1.4, or 1.5% w/v. In other embodiments, the concentration of the polyvalent cations in cross-linker solution is less than about 1.5, 1.4, 1.3, 1.2, 1.1, 1.0, 0.9, 0.8, 0.7, 0.6, 0.5, 0.4, 0.3, 0.2, 0.1, or 0.05% w/v. Combinations of these values to form various ranges are also possible, for example the concentration of the polyvalent cation in the hydrogel is about 0.1% w/v to about 1.3% w/v, 0.2% w/v to about 1.1% w/v, or about 0.3% w/v to about 0.7% w/v, for example about 0.5% w/v.

In some embodiments, the polyvalent cations in the cross-linker solution are divalent or trivalent cations or a mixture thereof.

In some embodiments, the polyvalent cations in cross-linker solution are divalent cations. The divalent cations may be selected from one or more Ca²⁺, Mg²⁺, Sr²⁺, Ba²⁺, Zn²⁺ and Be²⁺. In one embodiment, the cross-linker solution comprises Ca²⁺ cations. Sources for the Ca²⁺ ions used in cross-linker solution include, for example, calcium carbonate, calcium sulfate, calcium chloride, calcium phosphate, calcium tartrate, calcium nitrate, and calcium hydroxide. In one preferred embodiment, the cross-linker solution is calcium chloride (CaCl₂).

In some embodiments, the concentration of CaCl₂ is about 20 mM to about 500 mM. In some embodiments, the concentration of CaCl₂ is at least about 20, 30, 40, 50, 60, 70, 80, 90, 100, 150, 200, 250, 300, 350, 400, 450, or 500 mM. In other embodiments, the concentration of CaCl₂ is less than about 500, 450, 400, 350, 300, 250, 200, 150, 100, 90, 80, 70, 60, 50, 40, 30, 20, or 10 mM. Combinations of these concentrations to form various ranges are also possible, for example the concentration of CaCl₂ is about 10 mM to about 300 mM, 20 mM to about 200 mM, or about 40 mM to about 100 mM. In one embodiment, the concentration of CaCl₂ is about 40 mM to about 60 mM, for example about 50 mM.

In some embodiments, the concentration of CaCl₂ is about 0.05% and about 1.5% w/v. In some embodiments, the concentration of CaCl₂ is at least about 0.05, 0.1, 0.2, 0.3, 0.4, 0.5, 0.6, 0.7, 0.8, 0.9, 1.0, 1.1, 1.2, 1.3, 1.4, or 1.5% w/v. In other embodiments, the concentration of CaCl₂ is less than about 1.5, 1.4, 1.3, 1.2, 1.1, 1.0, 0.9, 0.8, 0.7, 0.6, 0.5, 0.4, 0.3, 0.2, 0.1, or 0.05% w/v. Combinations of these concentrations to form various ranges are possible, for example the concentration of CaCl₂ is about 0.1% w/v to about 1% w/v, 0.2% w/v to about 0.8% w/v, for example 0.5% w/v.

In some embodiments, the polyvalent cations in cross-linker solution are trivalent cations. The trivalent cations may be selected from one or more of Fe³⁺, Al³⁺, or Mn³⁺. In one embodiment, the cross-linker solution comprises Fe³⁺ cations. Sources for the Fe³⁺ ions used in cross-linker solution include, for example, iron (III) phosphate or iron(III) chloride. In some embodiments, the polyvalent cations in the cross-linker solution comprise both divalent cations and trivalent cations, both of which may be selected from one or more of the cations as described herein.

In one embodiment, the method of encapsulating or suspending a population of ciliate cells within a hydrogel further comprises a1) preparing a mixture comprising the suspension of ciliate cells and the hydrogel-forming polymer solution and adding the mixture of a1) to the cross-linker solution to form the hydrogel.

In one embodiment, one or more droplets of the mixture comprising the suspension of ciliate cells and the hydrogel-forming polymer solution (i.e. the mixture at a1) is added to the cross-linker solution to form the hydrogel.

For example, an aqueous solution containing the ciliate cells to be encapsulated (e.g. trophont ciliate cells) is mixed and suspended in the hydrogel-forming polymer solution (e.g. a sodium alginate solution), and added into the cross-linker solution (e.g. a CaCl₂ solution), which forms a plurality of hydrogel beads upon contact with ionic cross-linker (e.g. Ca²⁺), where the surface of the beads is cross-linked to form a hydrogel bead encapsulating the ciliate cells. This is due to the rapid cross-linking reaction which takes place, trapping the ciliate cells in the gel network.

In one embodiment, droplets of the mixture comprising the suspension of ciliate cells and the hydrogel-forming polymer solution may be added to the cross-linker solution by gravity (i.e. dropped into the cross-linker solution). In, other embodiments, droplets of the mixture comprising the suspension of ciliate cells and hydrogel-forming polymer solution may be sprayed into the cross-linker solution (e.g. via injection). In another embodiment, droplets of the mixture comprising the suspension of ciliate cells and the hydrogel-forming polymer solution is mixed with the cross-linker solution. It will be understood that, in some embodiments, regardless of the method of addition, the ciliate cells and hydrogel forming polymer solution are exposed to the cross-linker solution.

In one embodiment, the suspension of ciliate cells and the hydrogel-forming polymer solution is exposed to the cross-linker solution for less than about 20 minutes to form the hydrogel. This is also known as the “cross-linking” time. The longer the cross-linking time, the harder and more brittle the hydrogel becomes. In some embodiments, the cross-linking time is about 1 minute to about 10 minutes. In some embodiments, the cross-linking time is at least about 1, 2, 3, 4, 5, 6, 7, 8, 9, or 10 minutes. In other embodiments, the cross-linking time is less than about 10, 9, 8, 7, 6, 5, 4, 3, 2, or 1 minute. Combinations of these cross-linking times to form various ranges are also possible, for example about 1 minutes to about 7 minutes, 2 minutes to about 6 minutes, or 3 minutes to about 5 minutes. In one embodiment, the cross-linking time is about 5 minutes.

In some embodiments, the mixture at step a) or a1) further comprises magnesium sulfate. The present inventors have identified that, in some embodiments, magnesium sulfate, may trigger the encystment of trophont ciliate cells within the hydrogel. It will be appreciated that the embodiments provided above for the magnesium sulfate with regards to hydrogels also apply to the embodiments for the magnesium sulfate used in the mixture at step a) or a1).

In some embodiments, the method further comprises introducing optional additional components, such as an attractant or feeding stimulant. In one embodiment, the attractant or feeding stimulant is added into the mixture with the hydrogel-forming polymer solution. For example, the attractant or feeding stimulant is mixed with the hydrogel-forming polymer solution and suspension of ciliate cells prior to mixing with the cross-linking cation solution.

Alternatively, the attractant or feeding stimulant may be provided as a separate coating around the hydrogel. For example, hydrogels encapsulating or suspending ciliate cells are mixed with the attractant or feeding stimulant and a second hydrogel-forming polymer solution (which may be the same or different as the hydrogel-forming polymer solution of the hydrogel). This suspension is then mixed with the cross-linking cation solution which forms a polymer shell comprising the attractant or feeding stimulant around the hydrogel. Additional outer shells can be added to the hydrogel where appropriate, for example it will be appreciated that in this embodiment, the resulting hydrogel may be a core/shell bead comprising a hydrogel core encapsulating or suspending ciliate cells and an outer hydrogel shell, as described herein. The shell may comprise the attracting or feeding stimulant.

Another option to incorporate the attractant or feeding stimulant is to spray coat the hydrogel with a polymer containing the attractant or feeding stimulant. It will be appreciated that the embodiments provided above for the attractant or feeding stimulant with regard to the hydrogel also apply to the attractant or feeding stimulant incorporation/coating.

In some embodiments, the method further comprises incorporating one or more other optional additional components, such as a bait, pesticide, biocontrol agent, or other organisms such as pathogenic bacteria, fungal spores, or pathogenic nematodes into the hydrogel. In another embodiment, the method further comprises incorporating iron(III) phosphate (FePO₄). The embodiments described above in relation to the optional additional components of the hydrogel and/or composition also apply in relation to the optional additional components of the method.

In some embodiments, the hydrogel is washed to remove any excess cross-linking cation solution. The washing may be done by any suitable means, for example aspirating off the excess cross-linking cation solution followed by washing with water. In one embodiment, the water used to wash the hydrogels can be evaporated off prior to storage.

### Stabilisation of Encysted Ciliate Cells in Alternative Media

The present inventors have also identified an alternative media for stabilising encysted ciliate cells.

In one aspect or embodiment, there is provided a composition for stabilising encysted ciliate cells, the composition comprising encysted ciliate cells suspended in a buffer solution comprising magnesium ions.

In some embodiments, the buffer solution is a HEPES (4-(2-hydroxyethyl)-1-piperazineethanesulfonic acid) buffer solution or a phosphate buffer solution. In one embodiment, the buffer solution is a HEPES (4-(2-hydroxyethyl)-1-piperazineethanesulfonic acid) buffer solution.

In some embodiment, the concentration of HEPES in the HEPES buffer solution is at least about 1, 2, 5, 7, 10, 12, 15, 20 or 25 mM. In some embodiments, the concentration of HEPES in the HEPES buffer solution is less than about 25, 20, 15, 12, 10, 7, 5, 2 or 1 mM. Combinations of these concentrations are also possible, for example about 5 mM to about 25 mM, about 8 mM to 15 mM for example about 10 mM.

In some embodiments, buffer solution has a pH of about 6.0 to about 9.0, for example about 6.8 to about 8.2. In one embodiment, the buffer solution has a pH of about 7.

In one embodiment, the buffer solution comprises magnesium ions. In one embodiment, the buffer solution comprises magnesium sulfate (MgSO₄) or magnesium carbonate (MgCO₃). In one preferred embodiment, the buffer solution comprises magnesium sulfate (MgSO₄). It will be appreciated that when the magnesium sulfate or magnesium carbonate is dissolved in the buffer solution, magnesium ions (Mg²⁺) are present.

In one embodiment, the buffer solution is a HEPES buffered magnesium sulfate solution. In another embodiment, the buffer solution is a HEPES buffered magnesium carbonate solution.

In some embodiments, the concentration of the magnesium ions in the buffer solution is about 25 mM to about 100 mM. In some embodiments, the concentration of the magnesium ions in the buffer solution is at least about 25, 30, 35, 40, 50, 75, or 100, mM. In other embodiments, the concentration of the magnesium ions in the buffer solution is less than about 100, 75, 50, 40, 35, 30 or 25 mM. Combinations of these concentration values to form various ranges are also possible, for example the concentration of the magnesium ions in the buffer solution is about 25 mM to about 50 mM, for example about 25 mM. In one embodiment, the buffer solution comprises about 10 mM HEPES and 25 mM magnesium ions.

In one embodiment, the buffer solution comprises magnesium sulfate. In some embodiments, the concentration of the magnesium sulfate in the buffer solution is about 25 mM to about 100 mM. In some embodiments, the concentration of the magnesium sulfate in the buffer solution is at least about 25, 30, 35, 40, 50, 75, or 100, mM. In other embodiments, the concentration of the magnesium sulfate in the buffer solution is less than about 100, 75, 50, 40, 35, 30 or 25 mM. Combinations of these concentration values to form various ranges are also possible, for example the concentration of the magnesium sulfate in the buffer solution is about 25 mM to about 50 mM, for example about 25 mM. In one embodiment, the buffer solution comprises about 10 mM HEPES and 25 mM magnesium sulfate.

In one embodiment, the buffer solution is a HEPES buffer solution comprising magnesium sulfate, wherein the concentration of HEPES in the buffer solution is about 5 mM to 15 mM HEPES, wherein the pH of the buffer solution is about 6 to about 9, and the concentration of magnesium sulfate (MgSO₄) in the buffer solution is about 25 mM to about 50 mM.

In one embodiment, the buffer solution is a HEPES buffer solution comprising magnesium sulfate, wherein the concentration of HEPES in the buffer solution is about 10 mM HEPES, wherein the pH of the buffer solution is about 7, and the concentration of magnesium sulfate (MgSO₄) in the buffer solution is about 25 mM.

In some embodiments, buffer solution comprising encysted ciliate cells is stored at a temperature of about 20° C. to about 30° C., for example about 20° C. or 26° C.

The present inventors have also identified a method of stabilising encysted ciliate cells by dehydrating an aqueous solution comprising suspended soil particles (also referred to as an aqueous soil solution or a soil infusion water (SI-W)) which may be buffered (e.g. with HEPES) to form a buffered aqueous soil solution (SI-H) as described below) and pre-formed encysted ciliate cells. By dehydrating the encysted ciliate cells in the aqueous soil solution, encysted ciliate cells remained encysted and stable.

In one aspect or embodiment, there is provided a method of stabilising encysted ciliate cells, the method comprising dehydrating an aqueous solution comprising a population of encysted ciliate cells and suspended soil particles.

The aqueous solution may be dehydrated from a humidity under ambient conditions (e.g. an ambient humidity at 20° C. and atmospheric pressure) to a reduced humidity. For example, dehydrating the aqueous solution will result in a relative humidity of less than 100%. In some embodiments, the aqueous soil solution comprising the incubated trophont ciliate cells and suspended soil particles is dehydrated to a relative humidity of less than about 100%, 95%, 90%, 85%, 80%, 75%, 70%, 65%, 60%, 55%, 50%, 45%, or 40%, for example less than about 80%, 75%, or 70%. Combinations of these relative humidities are also possible, for example about 40% to about 75% relative to the ambient humidity. The dehydrated environment may be obtained by any suitable means, including for example using humidity chambers.

In some embodiments, the aqueous solution is dehydrated for at least about 0.5, 1, 2, 3, 4, 5, 8, 10, 12, 15, 18, 20, 24 or 30 days. In some embodiments, the aqueous solution is dehydrated at a temperature of about 20° C. to about 30° C., for example about 20° C.

In some embodiments, the aqueous soil solution may be buffered with a buffer solution to form a buffered aqueous soil solution (also referred to as a soil infusion buffer (SI-H). The buffer solution may be a HEPES (4-(2-hydroxyethyl)-1-piperazineethanesulfonic acid) buffer solution or a phosphate buffer solution. In one embodiment, the buffer solution is a HEPES (4-(2-hydroxyethyl)-1-piperazineethanesulfonic acid) buffer solution. In some embodiment, the concentration of HEPES in the HEPES buffer solution is at least about 1, 2, 5, 7, 10, 12, 15, 20 or 25 mM. In some embodiments, the concentration of HEPES in the HEPES buffer solution is less than about 25, 20, 15, 12, 10, 7, 5, 2 or 1 mM. Combinations of these concentrations are also possible, for example about 5 mM to about 25 mM, about 8 mM to 15 mM for example about 10 mM. In some embodiments, the buffered aqueous soil solution comprises a buffer solution having a pH of about 6.0 to about 9.0, for example about 6.8 to about 8.2. In one embodiment, the buffer solution has a pH of about 7. For example, the buffer solution has a pH of about 6.0 to about 9.0, for example about 6.8 to about 8.2, e.g. about pH 7.

In some embodiments, the aqueous soil solution or buffered aqueous soil solution comprises magnesium ions. In one embodiment, the aqueous soil solution or buffered aqueous soil solution comprises magnesium sulfate (MgSO₄) or magnesium carbonate (MgCO₃). In one preferred embodiment, the aqueous soil solution or buffered aqueous soil solution comprises magnesium sulfate (MgSO₄). It will be appreciated that when the magnesium sulfate or magnesium carbonate is dissolved in the aqueous soil solution or buffered aqueous soil solution, magnesium ions (Mg²⁺) are present.

In some embodiments, the concentration of the magnesium ions in the aqueous soil solution or buffered aqueous soil solution is about 15 μM to about 500 μM. In some embodiments, the concentration of the magnesium ions in the aqueous soil solution or buffered aqueous soil solution is at least about 15, 20, 30, 50, 100, 150, 200, 250, 300, 350, 400, 450, 500 or 1000 μM. In other embodiments, the concentration of the magnesium ions in the aqueous soil solution or buffered aqueous soil solution is less than about 1000, 500, 450, 400, 350, 300, 250, 200, 100, 50, 30, 20, or 15 μM. Combinations of these concentration values to form various ranges are also possible, for example the concentration of the magnesium ions in the aqueous soil solution or buffered aqueous soil solution is about 15 μM to about 1000 μM, about 20 μM to about 300 μM, about 30 μM to about 200 μM, or 50 μM to about 150 μM. In one embodiment, the concentration of magnesium ions in the aqueous soil solution or buffered aqueous soil solution is about 60 μM to about 65 μM, for example about 62.5 μM.

In one embodiment, the aqueous soil solution or buffered aqueous soil solution comprises magnesium sulfate. In some embodiments, the concentration of the magnesium sulfate in the aqueous soil solution or buffered aqueous soil solution is about 15 μM to about 500 μM. In some embodiments, the concentration of the magnesium sulfate in the aqueous soil solution or buffered aqueous soil solution is at least about 15, 20, 30, 50, 100, 150, 200, 250, 300, 350, 400, 450 500 or 1000 μM. In other embodiments, the concentration of the magnesium sulfate in the aqueous soil solution or buffered aqueous soil solution is less than about 1000, 500, 450, 400, 350, 300, 250, 200, 100, 50, 30, 20, or 15 μM. Combinations of these concentration values to form various ranges are also possible, for example the concentration of the magnesium sulfate in the aqueous soil solution or buffered aqueous soil solution is about 15 μM to about 1000 μM, about 20 μM to about 300 μM, about 30 μM to about 200 μM, or 50 μM to about 150 μM. In one embodiment, the concentration of magnesium sulfate in the aqueous soil solution or buffered aqueous soil solution is about 60 μM to about 65 μM, for example about 62.5 μM.

In some embodiments, the aqueous soil solution or buffered aqueous soil solution may further comprise a wetting agent and optionally one or more trace elements. In one embodiment, the aqueous soil solution or buffered aqueous soil solution further comprises the wetting agent Saturaid™.

The soil particles may comprise of any suitable soil, for example potting soil. In some embodiments, the soil particles may composted particles. In some embodiments, the soil particles may be pine bark particles or composted pine bark particles, or mixtures thereof.

The soil particles may have a suitable particle size. The soil particles may have an average particle size may be about 1 μm to about 200 μm. The soil particles may have an average particle size of at least about 1, 5, 10, 15, 20, 25, 30, 40, 50, 60, 80, 100, or 120 μm. The soil particles may have an average particle size of less than about 120, 100, 80, 60, 50, 40, 30, 25, 20, 15, 10, 5 or 1 μm. Combinations of average particle sizes are also possible, for example the soil particles may have an average particles size of about 5 μm to about 100 μm, or about 5 μm to about 60 μm. The soil particles may have an average particle size of less than about 60 μm. The particle size can be measured using an optical microscope or soil sieves.

In some embodiments, the aqueous soil solution or buffered aqueous soil solution comprises about 0.01% w/v to about 1% w/v soil particles based on the total volume of solution. In some embodiments, the aqueous soil solution or buffered aqueous soil solution comprises at least about 0.001, 0.01, 0.02, 0.05, 0.08, 0.1, 0.2, 0.5, or 1% w/v soil particles based on the total volume of solution. In some embodiments, the aqueous soil solution or buffered aqueous soil solution comprises less than 1, 0.5, 0.2, 0.1, 0.08, 0.05, 0.02, 0.01 or 0.001% w/v soil particles based on the total volume of solution. Combinations of these ranges are also possible, for example about 0.01% w/v to about 0.1% w/v soil particles based on the total volume of solution.

Alternatively or additionally, the aqueous solution or buffered aqueous solution may comprise one or more non-soil particles, such as algal cells, starch (e.g. corn starch), grains (e.g. rice), activated charcoal, magnesium silicate, polystyrene and dextrans (e.g. sulphopropyl and quaternary ammonia ethyl substituted dextrans) or bacterial cells.

In some embodiments, prior to incubating in the aqueous soil solution or buffered aqueous soil solution, the trophont cells were obtained via culturing T. rostrata in PPYE media (0.5% (w/v) proteose peptone (Oxoid LP0085), 0.5% (w/v) yeast extract (Oxoid LP0021), and 0.125% (w/v) glucose) or PP media (1% w/v Proteose Peptone (Oxoid LP0085) and 0.125% w/v glucose). In one embodiment, the trophont cells were obtained via culturing T. rostrata in PP media. Using trophont cells cultured in PP media may provide further advantages such as increased cyst resilience following encystment in the aqueous soil solution or buffered aqueous soil solution.

For one or both stabilisation methods described herein, in some embodiments, the population of encysted ciliate cells are provided at a concentration of about 1×10² cells/mL to about 1×10¹⁰ cells/mL. For example, the population of encysted ciliate cells may be provided at a concentration of about 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, or 1×10¹⁰ cells/mL. In some embodiments, the population of encysted ciliate cells are provided at a concentration of about 1×10⁴ cells/mL. Ranges of the these concentration values are also possible, for example about 1×10³ cells/mL to about 1×10⁵ cells/mL.

### Method of Chemically Inducing Encystment in Ciliate Cells

The present inventors have also identified a method of chemically inducing encystment of trophont ciliate cells into encysted ciliate cells. In one embodiment, this involved exposing the trophont ciliate cells to a buffer solution comprising one or more magnesium salts. As used herein, the term “buffer solution” refers to an aqueous solution consisting of a mixture of a weak acid and its conjugate base, or vice versa. The pH of the buffer solution changes very little when a small amount of strong acid or base is added to it. Buffer solutions are used as a means of keeping pH at a nearly constant value.

In some embodiments, the method comprises incubating a population of trophont ciliate cells in a buffer solution comprising magnesium ions.

In some embodiments, the buffer solution is a HEPES (4-(2-hydroxyethyl)-1-piperazineethanesulfonic acid) buffer solution or a phosphate buffer solution. In one embodiment, the buffer solution is a HEPES (4-(2-hydroxyethyl)-1-piperazineethanesulfonic acid) buffer solution. In another embodiment, the buffer solution is a Tris(tris(hydroxymethyl)aminomethane) buffer solution.

In some embodiments, the concentration of HEPES in the HEPES buffer solution is at least about 1, 2, 5, 7, 10, 12, 15, 20 or 25 mM. In some embodiments, the concentration of HEPES in the HEPES buffer solution is less than about 25, 20, 15, 12, 10, 7, 5, 2 or 1 mM. Combinations of these concentrations are also possible, for example about 5 mM to about 25 mM, about 8 mM to 15 mM for example about 10 mM.

In some embodiments, the buffer solution has a pH of about 6.0 to about 9.0, for example about 6.8 to about 8.2. In one embodiment, the buffer solution has a pH of about 7. For example, the buffer solution has a pH of about 6.0 to about 9.0, for example about 6.8 to about 8.2, for example about 7.

In one embodiment, the buffer solution comprises magnesium ions. In one embodiment, the buffer solution comprises magnesium sulfate (MgSO₄) or magnesium carbonate (MgCO₃). In one preferred embodiment, the buffer solution comprises magnesium sulfate (MgSO₄). It will be appreciated that when the magnesium sulfate or magnesium carbonate is dissolved in the buffer solution, magnesium ions (Mg²⁺) are present.

In one embodiment, the buffer solution is a HEPES buffered magnesium sulfate solution. In another embodiment, the buffer solution is a HEPES buffered magnesium carbonate solution. Without wishing to be bound by theory, it is believed that the presence of magnesium ions (Mg²⁺) may act as a trigger for encystment.

In some embodiments, the concentration of the magnesium ions in the buffer solution is about 15 μM to about 500 μM. In some embodiments, the concentration of the magnesium ions in the buffer solution is at least about 15, 20, 30, 50, 100, 150, 200, 250, 300, 350, 400, 450, 500 or 1000 μM. In other embodiments, the concentration of the magnesium ions in the buffer solution is less than about 1000, 500, 450, 400, 350, 300, 250, 200, 100, 50, 30, 20, or 15 μM. Combinations of these concentration values to form various ranges are also possible, for example the concentration of the magnesium ions in the buffer solution is about 15 μM to about 1000 μM, about 20 μM to about 300 μM, about 30 μM to about 200 μM, or 50 μM to about 150 μM. In one embodiment, the concentration of magnesium ions in the buffer solution is about 60 μM to about 65 μM, for example about 62.5 μM.

In one embodiment, the buffer solution comprises magnesium sulfate. In some embodiments, the concentration of the magnesium sulfate in the buffer solution is about 15 μM to about 500 μM. In some embodiments, the concentration of the magnesium sulfate in the buffer solution is at least about 15, 20, 30, 50, 100, 150, 200, 250, 300, 350, 400, 450 500 or 1000 μM. In other embodiments, the concentration of the magnesium sulfate in the buffer solution is less than about 1000, 500, 450, 400, 350, 300, 250, 200, 100, 50, 30, 20, or 15 μM. Combinations of these concentration values to form various ranges are also possible, for example the concentration of the magnesium sulfate in the buffer solution is about 15 μM to about 1000 μM, about 20 μM to about 300 μM, about 30 μM to about 200 μM, or 50 μM to about 150 μM. In one embodiment, the concentration of magnesium sulfate in the buffer solution is about 60 μM to about 65 μM, for example about 62.5 μM.

In one embodiment, the buffer solution is a HEPES buffer solution comprising magnesium sulfate, wherein the concentration of HEPES in the buffer solution is about 5 mM to 15 mM HEPES, wherein the pH of the buffer solution is about 6 to about 9, and the concentration of magnesium sulfate (MgSO₄) in the buffer solution is about 60 μM to about 65 μM.

In one embodiment, the buffer solution is a HEPES buffer solution comprising magnesium sulfate, wherein the concentration of HEPES in the buffer solution is about 10 mM HEPES, wherein the pH of the buffer solution is about 7, and the concentration of magnesium sulfate (MgSO₄) in the buffer solution is about 62.5 μM.

In some embodiments, the trophont ciliate cells are incubated with the buffer solution for about 12 hours to 48 hours, preferably about 24 hours. The trophont ciliate cells may be incubated with the buffer solution at a temperature of about 20° C. to about 30° C., for example about 26° C.

The present inventors have also identified a method of chemically inducing encystment of trophont ciliate cells into encysted ciliate cells using an aqueous solution comprising suspended soil particles (also referred to as an aqueous soil solution or a soil infusion water (SI-W)) which may be buffered (e.g. with HEPES) to form a buffered aqueous soil solution (SI-H) as described below. The aqueous solution comprises soil particles. By incubating trophont ciliate cells in the aqueous soil solution, one or more trophont ciliate cells undergo encystment to form encysted ciliate cells.

In one aspect or embodiment, there is provided a method of inducing the encystment of ciliate cells, the method comprising incubating a population of trophont ciliate cells in an aqueous solution comprising suspended soil particles (i.e. an aqueous soil solution), wherein the trophont ciliate cells undergo encystment to form one or more encysted ciliate cells.

In some embodiments, the aqueous soil solution may be buffered with a buffer solution to form a buffered aqueous soil solution (also referred to as a soil infusion buffer (SI-H). The buffer solution may be a HEPES (4-(2-hydroxyethyl)-1-piperazineethanesulfonic acid) buffer solution or a phosphate buffer solution. In one embodiment, the buffer solution is a HEPES (4-(2-hydroxyethyl)-1-piperazineethanesulfonic acid) buffer solution. In some embodiment, the concentration of HEPES in the HEPES buffer solution is at least about 1, 2, 5, 7, 10, 12, 15, 20 or 25 mM. In some embodiments, the concentration of HEPES in the HEPES buffer solution is less than about 25, 20, 15, 12, 10, 7, 5, 2 or 1 mM. Combinations of these concentrations are also possible, for example about 5 mM to about 25 mM, about 8 mM to 15 mM for example about 10 mM.

In some embodiments, the buffered aqueous soil solution comprises a buffer solution having a pH of about 6.0 to about 9.0, for example about 6.8 to about 8.2. In one embodiment, the buffer solution has a pH of about 7. For example, the buffer solution has a pH of about 6.0 to about 9.0, for example about 6.8 to about 8.2, e.g. about pH 7.

In some embodiments, the aqueous soil solution or buffered aqueous soil solution comprises magnesium ions. In one embodiment, the aqueous soil solution or buffered aqueous soil solution comprises magnesium sulfate (MgSO₄) or magnesium carbonate (MgCO₃). In one preferred embodiment, the aqueous soil solution or buffered aqueous soil solution comprises magnesium sulfate (MgSO₄). It will be appreciated that when the magnesium sulfate or magnesium carbonate is dissolved in the aqueous soil solution or buffered aqueous soil solution, magnesium ions (Mg²⁺) are present.

In some embodiments, the concentration of the magnesium ions in the aqueous soil solution or buffered aqueous soil solution is about 15 μM to about 500 μM. In some embodiments, the concentration of the magnesium ions in the aqueous soil solution or buffered aqueous soil solution is at least about 15, 20, 30, 50, 100, 150, 200, 250, 300, 350, 400, 450, 500 or 1000 μM. In other embodiments, the concentration of the magnesium ions in the aqueous soil solution or buffered aqueous soil solution is less than about 1000, 500, 450, 400, 350, 300, 250, 200, 100, 50, 30, 20, or 15 μM. Combinations of these concentration values to form various ranges are also possible, for example the concentration of the magnesium ions in the aqueous soil solution or buffered aqueous soil solution is about 15 μM to about 1000 μM, about 20 μM to about 300 μM, about 30 μM to about 200 μM, or 50 μM to about 150 μM. In one embodiment, the concentration of magnesium ions in the aqueous soil solution or buffered aqueous soil solution is about 60 μM to about 65 μM, for example about 62.5 μM.

In one embodiment, the aqueous soil solution or buffered aqueous soil solution comprises magnesium sulfate. In some embodiments, the concentration of the magnesium sulfate in the aqueous soil solution or buffered aqueous soil solution is about 15 μM to about 500 μM. In some embodiments, the concentration of the magnesium sulfate in the aqueous soil solution or buffered aqueous soil solution is at least about 15, 20, 30, 50, 100, 150, 200, 250, 300, 350, 400, 450 500 or 1000 μM. In other embodiments, the concentration of the magnesium sulfate in the aqueous soil solution or buffered aqueous soil solution is less than about 1000, 500, 450, 400, 350, 300, 250, 200, 100, 50, 30, 20, or 15 μM. Combinations of these concentration values to form various ranges are also possible, for example the concentration of the magnesium sulfate in the aqueous soil solution or buffered aqueous soil solution is about 15 μM to about 1000 μM, about 20 μM to about 300 μM, about 30 μM to about 200 μM, or 50 μM to about 150 μM. In one embodiment, the concentration of magnesium sulfate in the aqueous soil solution or buffered aqueous soil solution is about 60 μM to about 65 μM, for example about 62.5 μM.

In some embodiments, the trophont ciliate cells are incubated with the aqueous soil solution or buffered aqueous soil solution for about 12 hours to 48 hours, preferably about 24 hours. The trophont ciliate cells may be incubated with the aqueous soil solution or buffered aqueous soil solution at a temperature of about 20° C. to about 30° C., for example about 26° C.

In some embodiments, the aqueous soil solution or buffered aqueous soil solution may further comprise a wetting agent and optionally one or more trace elements. In one embodiment, the aqueous soil solution or buffered aqueous soil solution further comprises the wetting agent Saturaid™.

The soil particles may comprise of any suitable soil, for example potting soil. In some embodiments, the soil particles may composted particles. In some embodiments, the soil particles may be pine bark particles or composted pine bark particles, or mixtures thereof. The term “composted” refers to particles (e.g. pine bark particles) obtained from a potting mix. For example, a buffered aqueous soil solution may be obtained by infusing a potting mix (e.g. Australian Growing Solutions) in water and subsequently autoclaved. The resulting infusion comprises fine bark particles and one or more solutes from the bark particles which have leached out during infusion.

The soil particles may have a suitable particle size. The soil particles may have an average particle size may be about 1 μm to about 200 μm. The soil particles may have an average particle size of at least about 1, 5, 10, 15, 20, 25, 30, 40, 50, 60, 80, 100, or 120 μm. The soil particles may have an average particle size of less than about 120, 100, 80, 60, 50, 40, 30, 25, 20, 15, 10, 5 or 1 μm. Combinations of average particle sizes are also possible, for example the soil particles may have an average particles size of about 5 μm to about 100 μm, or about 5 μm to about 60 μm. The soil particles may have an average particle size of less than about 60 μm. The particle size can be measured using an optical microscope or soil sieves. Without wishing to be bound by theory, further advantages may be provided from incubating trophont ciliate cells in an aqueous soil solution comprising smaller soil particles (e.g. less than 60 μm) stimulates food vacuoles within the ciliate thus promoting encystment.

In some embodiments, the aqueous soil solution or buffered aqueous soil solution comprises about 0.01% w/v to about 1% w/v soil particles based on the total volume of solution. In some embodiments, the aqueous soil solution or buffered aqueous soil solution comprises at least about 0.001, 0.01, 0.02, 0.05, 0.08, 0.1, 0.2, 0.5, or 1% w/v soil particles based on the total volume of solution. In some embodiments, the aqueous soil solution or buffered aqueous soil solution comprises less than 1, 0.5, 0.2, 0.1, 0.08, 0.05, 0.02, 0.01 or 0.001% w/v soil particles based on the total volume of solution. Combinations of these ranges are also possible, for example about 0.01% w/v to about 0.1% w/v soil particles based on the total volume of solution.

Alternatively or additionally, the aqueous solution or buffered aqueous solution may comprise one or more non-soil particles, such as algal cells, starch (e.g. corn starch), grains (e.g. rice), activated charcoal, magnesium silicate, polystyrene and dextrans (e.g. sulphopropyl and quaternary ammonia ethyl substituted dextrans) or bacterial cells.

In some embodiments, prior to incubating in the aqueous soil solution or buffered aqueous soil solution, the trophont cells were obtained via culturing T. rostrata in PPYE media (0.5% (w/v) proteose peptone (Oxoid LP0085), 0.5% (w/v) yeast extract (Oxoid LP0021), and 0.125% (w/v) glucose) or PP media (1% w/v Proteose Peptone (Oxoid LP0085) and 0.125% w/v glucose). In one embodiment, the trophont cells were obtained via culturing T. rostrata in PP media. Using trophont cells cultured in PP media may provide further advantages such as increased cyst resilience following encystment in the aqueous soil solution or buffered aqueous soil solution.

The present inventors have also identified a method of inducing encystment of trophont ciliate cells into encysted ciliate cells by dehydrating an aqueous solution comprising suspended soil particles and trophont ciliate cells. By dehydrating the trophont ciliate cells in the aqueous soil solution, one or more trophont ciliate cells undergo encystment to form encysted ciliate cells. In embodiment, the method further comprises dehydrating the aqueous solution comprising the incubated trophont ciliate cells and suspended soil particles. The aqueous solution may be dehydrated from a humidity under ambient conditions (e.g. an ambient humidity at 20° C. and atmospheric pressure) to a reduced humidity. For example, dehydrating the aqueous solution will result in a relative humidity of less than 100%. In some embodiments, the aqueous soil solution comprising the incubated trophont ciliate cells and suspended soil particles is dehydrated to a relative humidity of less than about 100%, 95%, 90%, 85%, 80%, 75%, 70%, 65%, 60%, 55%, 50%, 45%, or 40%, for example less than about 80%, 75%, or 70%. Combinations of these relative humidities are also possible, for example about 40% to about 75% relative to the ambient humidity. The dehydrated environment may be obtained by any suitable means, including for example using humidity chambers. The humidity levels may be measured by any routine means including a humidity monitor or hygrometer, such as a gravimetric hygrometer.

In some embodiments, the aqueous solution is dehydrated for at least about 0.5, 1, 2, 3, 4, 5, 8, 10, 12, 15, 18, 20, 24 or 30 days. In some embodiments, the aqueous solution is dehydrated at a temperature of about 20° C. to about 30° C., for example about 20° C. For one or both encystment methods described herein, in some embodiments, the population of trophont ciliate cells are provided at a concentration of about 1×10² cells/mL to about 1×10¹⁰ cells/mL. For example, the population of trophont ciliate cells may be provided at a concentration of about 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, or 1×10¹⁰ cells/mL. In some embodiments, the population of trophont ciliate cells are provided at a concentration of about 1×10⁴ cells/mL. Ranges of the these concentration values are also possible, for example about 1×10³ cells/mL to about 1×10⁵ cells/mL. In some embodiments, the trophont ciliate cells are young trophont ciliate cultures, for example have undergone less than 10, 9, 8, 7, 6, 5, 4, 3, 2 or 1 subcultures. The number of passages give an indication of the number of cell generations that have occurred since cells last went through autogamy. Trophont ciliate cultures that have undergone less than 10 subcultures are considered to contain “young” trophont cells.

In some embodiments, the chemically induced encysted ciliate cells are subsequently transferred to fresh nutrient medium where they undergo excystment to form theront ciliate cells. Alternatively, the encysted ciliate cells may be encapsulated or suspended within a hydrogel as described herein. Alternatively, the encysted ciliate cells may be stabilised and stored in a buffered environment as described herein. Alternatively, the encysted ciliate cells may be stabilised and stored by dehydration as described herein.

### Storage, Stability and Viability of Encapsulated/Suspended Ciliate Cells

The compositions comprising the hydrogel encapsulating or suspending ciliate cells are stable and the ciliate cells remained viable during storage. In one embodiment, depending on the choice of hydrogel, the ciliate cells within the hydrogel remained as encysted ciliate cells during storage. In another embodiment, depending on the choice of hydrogel, the ciliate cells within the hydrogel remained as trophont ciliate cells.

For example, the present inventors discovered that, in some embodiments, trophont ciliate cells that were encapsulated within a hydrogel (such as an alginate hydrogel bead) underwent encystment within the hydrogel to form encysted ciliate cells, and remained as encysted ciliate cells during storage. In another example, trophont ciliate cells that were suspended within a hydrogel (such as a carboxymethylcellulose liquid hydrogel) remained as trophont ciliate cells during storage. Additionally, it was also identified that if pre-formed encysted cells were suspended or encapsulated within the same hydrogel, they also remained as encysted ciliate cells during storage. This demonstrates that ciliate cells at different developmental stages can be stored and remain stable when suspended or encapsulated in the hydrogels of the present invention.

In some embodiments, nearly all of the encysted ciliate cells encapsulated or suspended within the hydrogel remained as encysted ciliate cells and viable for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 12 weeks, 15 weeks, or 20 weeks, highlighting the effect hydrogel encapsulation has on ciliate cell stability.

In some embodiments, the washed hydrogel is stored in a sealed container.

In one embodiment, the ciliate cells encapsulated or suspended within the hydrogel can be stored under ambient conditions (i.e. in the dark, room temperature). For example, the storage temperature may be about 1° C. to about 30° C. In some embodiments, the storage temperature may be at least about 1, 2, 3, 4, 5, 6, 7, 8, 9, 10, 12, 14, 16, 18, 20, 22, 24, 26, 28, or 30° C. In other embodiments, the storage temperature may be less than about 30, 28, 26, 24, 22, 20, 18, 16, 14, 12, 10, 9, 8, 7, 6, 5, 4, 3, or 2° C. Combinations of these storage temperatures to form various ranges are also possible, for example, about 3° C. to about 25° C., or about 4° C. to about 28° C.

For example, the present inventors have identified that, in some embodiments, encysted ciliate cells encapsulated within hydrogels remained encysted and viable after 8 weeks storage at 20° C. These encysted cells remained viable and following release from the hydrogel, the encysted cells were able to excyst into theront cells and establish new populations in culture, highlighting the improved storage properties of the hydrogels of the present invention.

In another embodiment, the ciliate cells encapsulated or suspended within the hydrogel can be stored in the dark.

In some embodiments, the ciliate cells encapsulated or suspended within the hydrogel can be stored with minimal to no additional moisture.

In some embodiments, the encapsulated or suspended ciliate cells can subsequently be released from the hydrogel into the external environment. For example, the hydrogels can be suspended/soaked in water which dilutes and dissolves the cross-linking cations within the hydrogel thus softening the hydrogel structure allowing for the encysted or encapsulated cells to be released to the external environment. Alternatively, the hydrogels can be suspended in an aqueous solution comprising a chelation agent which competitively binds to the cross-linking cations thereby disrupting the hydrogel matrix. Examples of suitable chelation agents include sodium citrate, EDTA or phosphate. Other suitable mediums that can release encysted ciliate cells from the hydrogel include PPYE medium, or enzymes such as alginate lyase.

In some embodiments, the encysted cells encapsulated or suspended within the hydrogel are able to undergo excystment to form theront ciliate cells upon release from the hydrogel. For example, when the hydrogel encapsulating or suspending encysted ciliate cells is suspended in fresh media (e.g. sodium citrate buffer or PPYE medium), the encysted ciliate cells are gradually released which then undergo excystment to form theront ciliate cells. The theront ciliate cells can then mature into a healthy new trophont cell cultures.

Isolated Strain of T. rostrata

The present inventors have also identified an isolated strain of T. rostrata deposited under PTA-126056 on 13 Aug. 2019 at the American Type Culture Collection.

In one embodiment, the isolated strain of T. rostrata comprises a mitochondrial genome which has a nucleotide sequence as shown in SEQ ID NO:1. In some embodiments, the mitochondrial genome has a nucleotide sequence which is at least 99%, 98%, 97%, 96%, 95%, 94%, 93%, 92%, 91%, or 90% identical to SEQ ID NO:1.

In another embodiment, the isolated strain of T. rostrata comprises a cox1 gene which has a nucleotide sequence as shown in SEQ ID NO:7. In some embodiments, the cox1 gene has a nucleotide sequence which is at least at least 99%, at least 99.5% or 99.9% identical to SEQ ID NO:7.

In an embodiment, the % identity of a polynucleotide is determined by GAP (Needleman and Wunsch, 1970) analysis (GCG program) with a gap creation penalty=5, and a gap extension penalty=0.3. Preferably, the GAP analysis aligns two sequences over their entire length.

In one embodiment, there is provided a composition comprising or consisting of the T. rostrata strain as described herein and optionally one or more acceptable carriers. The carrier may be selected from the acceptable carriers as described herein in relation to the hydrogel. In one embodiment, there is provided a composition comprising the T. rostrata strain as described herein encapsulated or suspended in an alginate hydrogel. In one embodiment, there is provided a composition comprising the T. rostrata strain as described herein encapsulated or suspended in a CMC hydrogel. In one embodiment, there is provided a composition comprising the T. rostrata strain as described herein encapsulated or suspended in a CMC/alginate core-shell hydrogel.

Method of Infecting or Colonising a Pest Species with a Ciliate

The present inventors have also identified that theront cells are the infective form of T. rostrata which were more effective at killing or reducing the fitness of slugs faster than trophont ciliate cells. Therefore, the compositions of the present invention can be used to transport and deliver viable and stable encysted ciliate cells or trophont ciliate cells to an area affected or likely to be affected by pests (e.g. slugs or snails), where once the encapsulated/suspended cells are released from within the hydrogel they can undergo excystment to form the infective theront ciliate cells which are released into the environment and infect pests. Therefore, in some embodiments, the compositions, ciliate cells and/or isolated strains of T. rostrata as described herein can be dispersed in the environment for infection or colonisation of pests. In one embodiment, the hydrogels encapsulating or suspending ciliate cells as described herein can be dispersed in the environment for infection or colonisation of pests.

In a related aspect or embodiment, there is provided a method of infecting or colonising a pest species with a ciliate, the method comprising applying to an area affected or likely to be affected by a pest species a strain of T. rostrata as described herein or a composition comprising the strain of T. rostrata as described herein.

As used herein, the term “colonisation” or variants thereof refers to the entry of the protist to an external facing tissue or orifice of the pest such as the sub-mantle tissue lining or gut tissue following ingestion of the hydrogel or protists released therefrom. In one embodiment, the pest species may eat through the hydrogel and release the encapsulated or suspended ciliate cells. For example, the present inventors have discovered that, in some embodiments, when the hydrogel compositions of the present invention are in the presence of slugs in an infection experiment, the slugs are able to eat through the beads and release the ciliate cells which can infect the slug.

In some embodiments, the compositions can be applied to agriculture, aquaculture and/or horticulture. In one embodiment, the compositions, ciliate cells and/or isolated strains may be applied to an area of soil affected by a pest (such as a slug or a snail). For example, in relation to the hydrogel compositions, the hydrogel may disintegrate in the moist soil environment and release the ciliate cells into the soil. If the cells released from the hydrogel are encysted ciliate cells, then the encysted ciliate cells released from the hydrogel may subsequently undergo excystment within the soil into theront ciliate cells which are infective and can infect various pest species living in, on or around the soil area. If the cells released from the hydrogel are trophont ciliate cells, then once released, the trophont ciliate cells may encyst in the soil environment to form encysted ciliate cells, which can then subsequently excyst to form the infective theront ciliate cells, as per the life cycle in FIG. 1 or can invade the pest animal tissue directly. The released ciliate cells may also be ingested by the pest, and undergo transformation within the pest to form theront ciliate cells. In yet another embodiment, if the released ciliate cells are theront ciliate cells, these may go on to infect the pest species.

In other embodiments, the hydrogel compositions could be applied to an area and subsequently wetted (e.g. by the rain, sprinkler, inundation, or drip irrigation etc.) wherein the water environment disrupts the cross-linking of the hydrogel. The hydrogel may then disintegrate in the moist environment and release the ciliate cells, which can go on to form theront ciliate cells, and subsequently infect pest species.

Other areas that may be affected by pests that the compositions, ciliate cells and/or isolated strains of the present invention can be applied to include farms, gardens, crops, nurseries, pastures, fields, greenhouses, shadehouses, hydroponic nurseries.

In another embodiment, the compositions may be added to a container with holes to allow access for pest species, for example commercial traps (refuges) currently on the market for pest control, such as slug and snail traps. Without wishing to be bound by theory, placing the hydrogels in such traps may expose the pests long enough for them to get infected with the encapsulated/suspended ciliate cells which then kills them, or even if the pests manage to leave the trap, the infected pest would disperse the ciliates as they migrate around the adjacent area, thus spreading the ciliate infection to other pests in the area. The trap can then be refilled with more hydrogels as the pest species consumes them. Other advantages may be provided by such using traps to deliver the hydrogels to pest species, such as portability to different garden areas as damage to plants is observed indicating the presence of pests requiring control. It will be appreciated that other delivery methods are also envisaged using the hydrogels and the above examples are not to be considered limiting.

In some embodiments, the compositions, ciliate cells and/or isolated strains of T. rostrata of the present invention may comprise or be further mixed with one or more acceptable carriers. The acceptable carrier may be an agriculturally or horticulturally suitable carrier. As used herein, an “acceptable carrier” and/or an “agriculturally suitable carrier” and/or a “horticulturally suitable carrier” is any carrier on which can facilitate the transport of the compositions, ciliate cells and/or isolated strains to an area affected or likely to be affected by a pest species (such as an invertebrate), and which is otherwise suitable for agricultural or horticultural use. Any such suitable acceptable carrier can be used, including but not limited to seeds, seed coats, granular carriers, liquid slurry carriers, and liquid suspension carriers.

Suitable agriculturally or horticulturally acceptable carriers include fillers, solvents, excipients, surfactants, suspending agents, spreaders/stickers (adhesives), antifoaming agents, dispersants, wetting agents, drift reducing agents, auxiliaries, adjuvants or a mixture thereof. For example, the agriculturally or horticulturally acceptable carrier may be selected from the group consisting of a filler stimulant, an anti-caking agent, a wetting agent, an emulsifier, and an antioxidant, for example said composition comprises at least one of each of a filler stimulant, an anti-caking agent, a wetting agent, an emulsifier, and an antioxidant.

In one embodiment solid carriers include but are not limited to mineral earths such as silicic acids, silica gels, silicates, talc, kaolin, attapulgus clay, limestone, lime, chalk, bole, loess, clay, bentonite, dolomite, diatomaceous earth, aluminas calcium sulfate, magnesium sulfate, magnesium oxide, peat, humates, ground plastics, fertilizers such as ammonium sulfate, ammonium phosphate, ammonium nitrate, and ureas, and vegetable products such as grain meals, bark meal, wood meal, and nutshell meal, cellulosic powders, seaweed powders, peat, talc, carbohydrates such as mono-saccharides and di-saccharides, starch extracted from corn or potato or tapioca, chemically or physically altered corn starch and the like. As solid carriers for the compositions, cells and strains of the present invention, the following are suitable as carriers: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Such solid compositions may, if desired, contain one or more compatible wetting, dispersing, emulsifying or colouring agents which, when solid, may also serve as a diluent.

The acceptable carrier preferably has a sufficient shelf life, and preferably assists in the dispersion of the compositions, ciliate cells and/or isolated strains to an area affected or likely to be affected by a pest species (such as an invertebrate).

The pest species can be an invertebrate or a vertebrate. In one embodiment, the vertebrate is a lower vertebrate.

In one embodiment, the pest species is an invertebrate. The invertebrate may be a mollusc or arthropod, such as a dipteran (e.g. a mosquito). In another embodiment, the pest species is a vertebrate, for example a fish species. In a preferred embodiment, the invertebrate is a mollusc, for example a Gastropod.

The Gastropod may be a snail or a slug. The slugs and snails to be controlled in include all land-dwelling slugs and snails, for example those which occur as polyphagus pests in agricultural and horticultural crops. Agriculturally and horticulturally problematic slug and snail types are, for example, slugs such as the invasive Arion ater group such as A ater, A rufus, and A vulgaris). Other non-limiting examples of slugs to be controlled include Ambigolimax valentianus, Deroceras invadens, Limacus flavus, Deroceras reticulatum, and grey field slugs.

### EXAMPLES

### Example 1—Materials and Methods

### Solutions and Media

All media and solutions were prepared fresh using laboratory grade water and sterilised by autoclaving at 121° Celsius for 20 minutes unless otherwise noted. SPP medium consisted of 2% proteose peptone (Oxoid), 0.1% w/v yeast extract, 0.2% w/v glucose and 33 μM FeCl₃ with antibiotics (200 units/ml of penicillin, 200 μg/ml of streptomycin, and 0.5 μg/mL of amphotericin B. The FeCl₃ and antibiotics were added after autoclaving to cooled media. PPYE media consisted of 0.5% (w/v) proteose peptone (Oxoid LP0085), 0.5% (w/v) yeast extract (Oxoid LP0021), and 0.125% (w/v) glucose. PP medium was PPYE without yeast extract (1% w/v Proteose Peptone (Oxoid LP0085) and 0.125% w/v glucose). All manipulation of T. rostrata cells were performed aseptically. RM9 was composed of 0.5% (w/v) Proteose Peptone (Oxoid LP0085), 0.5% w/v Tryptone (Bacto™ tryptone BD REF 211705), 0.02 g w/v K₂HPO₄, 0.1% w/v glucose, 0.01%; w/v liver extract (MP liver concentrate NF #×1 MF cat no 900377).

Isolation of T. rostrata TRAUS

Strain TRAUS was isolated from an egg laid by a F1 laboratory-reared D. reticulatum whose parents were collected from Melbourne, Australia. The egg was surface sterilised in 0.01% v/v hypochlorite for 5 min then washed several times in sterile distilled water to remove the hypochlorite. The egg was aseptically opened using a needle to release the ciliates into water and then immediately transferred to 10 ml of SPP media in a 25 cm² tissue culture flask with a vented lid (IWAKI). Pure cultures of T. rostrata TRO1 were obtained from the American Type Culture Collection (ATCC®PRA326™).

Once established, the cultures were maintained in PPYE, PP or ATCC 357 media without antibiotics at 20° C. in the dark. Subcultures were made fortnightly by doing a 1:20 dilution into fresh medium.

Identification of T. rostrata TRAUS

The identification of T. rostrata TRAUS was made by microscopic examination of cells using a scanning electron microscope. Actively growing cultures were prepared for scanning electron microscopy as follows. Cells in 10 mL of culture were collected by centrifugation and rinsed three times with sterile distilled water and resuspended in 1 mL of sterile distilled water, then fixed by the addition of 100 μL of 25% glutaraldehyde to give a final concentration of 2.5% in solution. The ciliates were fixed for 10 minutes at room temperature and 200 μL aliquots were pipetted onto glass coverslips which were coated with a 0.1% solution of polyethyleneimine and incubated for 1 hour, to allow fixed cells to adhere to the coverslips. Following incubation, the excess supernatant was drained, and coverslips with adhered ciliates were dehydrated in increasing concentrations of ethanol; 10, 30, 50, 70, 90 and 100% ethanol in water for 60 minutes each step. The coverslips were dried in a Balzers CPD030 critical point dryer (Balzers, Liechtenstein, Germany) and mounted onto 25 mm aluminium stubs with double sided carbon tabs. The coverslips were coated with gold using a Xenosput sputter coater (Dynavac, Wantirna South, Australia). The ciliates on coverslips were imaged with the Philips XL30 field emission scanning electron microscope (Philips, Eindhoven, Netherlands) at a voltage of 2.0 kV and a spot size of 2. Measurements were made with Image J.

Molecular Identification of T. rostrata TRAUS

Genomic DNA was extracted from whole trophonts cells using a Promega Wizard genome DNA purification kit. The sequencing library, prepared using the Illumina TruSeq kit, was enriched using the KAPA enzyme (Millennium Science) and sequenced using Illumina MiSeq™. The raw data was filtered using the mitochondrial genome (mt genome) of T. pigmentosa as a reference and the resulting reads were de novo assembled with Unicycler version 0.4.7 followed by gap filling. Annotation was done using Geneious Prime version 1.3 (Kearse et al., 2012) with reference to published Tetrahymena mt genomes.

The T. rostrata TRAUS cox1 gene sequencing was performed using MAFFT version 7.388 (Katoh et al, 2013) and Bayesian phylogenetic inference was performed using a Markov chain Monte Carlo (MCMC) analysis in Mr Bayes version 3.2.6 using a 11,000,000 MCMC generation chain length with consensus trees generated using the 50% majority rule criterion and the final 90% of trees generated by (BI) after a burn-in of 100,000 generations. The mt genome was 47,235 bp linear DNA and had a GC % of 21.8%.

### Encystment in Tris Buffer

The method was essentially as described by Kaczanowski et al. (2016). Briefly, ciliate cells from actively growing cultures (˜1×10⁵ cell/ml) were harvested by centrifugation (800 g, 10 min), washed in 2× culture volumes of 10 mM Tris-HCl pH 7.4 and resuspended in the same buffer a density of about 5×10⁴ cells/ml. The cells were then dispensed in flat-bottomed tissue culture suspension plates and incubated at 26° C. in the dark.

### Encystment in Soil Infusion Buffer (SI-H)

A soil infusion was prepared, according to a modified protocol by Segade et al. (2016). 100 g of Plugger 111-Seedraising Mix (Australian Growing Solution 18) was suspended in 1.2 litres of Milli Q ultrapure water. The suspension was maintained in agitation for 15 min at room temperature (20° C.) and then large particles were removed by sieving and fine particles were removed by centrifugation at 300 g for 10 min. The decanted supernatant was sterilised by autoclaving and HEPES buffer pH7 was added to a final concentration of 10 mM. T. rostrata cells were harvested from actively growing cultures as above, washed in soil infusion buffer, transferred to tissue culture plates or flasks and incubated in the dark at 20° C. or 26° C.

The effect the culture media used to grow trophonts had on encystment in soil infusion was also investigated. The ability of trophonts grown in RM9, PPYE and PP to form cysts in soil infusion buffer at 26° C. over 6 days was investigated.

### Preparation of Theronts Suspensions

Cysts were prepared using buffered soil infusion at 26° C. After 24 hours, 99-100% of cells were cysts and they formed an adherent lawn on the basis of the plate. Any non-adhered cysts or residual swimming cells were removed by gently washing the lawn with buffered soil infusion. The cysts were then incubated for a further five to seven days at 20° C. to allow the cysts to complete autogamy (i.e. mature) and become primed for excystment into theronts.

The cysts were then detached from the plastic by gentle pipetting at 20° C. Excystment occurred readily and after 1 hr 85-90% of the cells were excysted. For the bioassays the theronts were purified to 100% through harvesting the cells by gentle centrifugation (300 g, 10 min) and then allowing the theronts to swim out of the cell pellet into the supernatant for 2 hrs.

D. reticulatum Colony

A colony of laboratory-reared D. reticulatum was established from individuals collected from Melbourne, Australia. D. reticulatum slugs were reared in 4 L, non-airtight, plastic containers lined with a folded moist cloth (CHUX® Superwipes®). The base of each box contained approx. 2 cm of damp of seed propagation soil (Plugger 111-Seedraising Mix (Australian Growing Solutions)). Slugs were provided with fresh Chinese cabbage and sliced carrot on a plastic dish twice weekly. Small pieces of cuttlebone were provided as a calcium source and the diet was supplemented occasionally with small amounts of dried cat food. The boxes were kept at 10-12° C. with a 12 hour light/dark photoperiod and were cleaned weekly. Eggs laid in soil were collected weekly and hatched containers at 16° C. before being moved to rearing boxes.

### Slug Infection Assays

Slugs for infection experiments were selected from young slugs approximately 0.8 cm long or in some cases 1 to 1.5 cm long. Unless specified, infections were performed in 28 ml plastic tubes containing 3 g of potting soil, 1.2 to 1.4 ml of water and ˜1 cm² of Chinese cabbage as food. The tubes were closed with cellulose acetate stoppers and kept in groups according to treatment type in plastic boxes lined with moist cloth to create a humid atmosphere. Boxes were kept in the laboratory at a temperature of 17-20° C. unless otherwise specified.

### Petri Dish Experiments

Healthy adults were placed in groups of three in boxes lined with damp paper towel for the bioassay. Slugs were exposed to TRO1 or TRAUS cultured at 25° C. in ATCC medium (5×10³ cells/mL in water applied over the body and on food) or water only. There were 15 replicates for each treatment. Slugs were kept at 16° C. with a 12 hour light dark period. Dead slugs and eggs were removed at each inspection and food was replaced.

### Neonate Experiments

Slug eggs were collected and treated for five days with metronidazole (2.5 μg/mL) added daily to eliminate contaminants. Eggs were then washed thoroughly with water and transferred to fresh unvented petri dishes. Eggs were incubated and eggs at the same Stage V of development were selected (Carrick, 1939). All slugs used for the assay hatched within 48 hours and were randomly assigned into petri dishes (10 slugs per petri dish) to ensure that eggs in a given treatment were sourced from a number of adults. Slugs were allowed to acclimatise to the fresh arena for 24 h prior to infection with ciliates. Ciliate suspension (5×10³ cells/mL) was dripped onto the backs of the slugs on the wet filter paper and the chambers were incubated for a further 24 h before a small piece of lettuce was added to the petri dish as food. Live slugs were counted regularly and fresh food was added when required. Survival of slugs was analysed using the binary logistic regression model in Minitab® v. 17.1.0.

### Tube Experiments

T. rostrata TRAUS trophonts grown in PPYE media and theronts derived from cysts made in soil infusion were used in slug infection assays. Plastic 28 ml, wide mouth tube containing ˜3 g of soil (Plugger 111-Seedraising Mix (Australian Growing Solutions)) and 1.2 to 1.4 ml of water and stoppered with cellulose acetate plugs were used in all tests. One to five young, laboratory-reared D. reticulatum slugs (<0.8 cm long) were placed in each tube along with a 1 cm² piece of Chinese cabbage for food. T. rostrata TRAUS trophonts or theronts (10⁴ cells per tube) were added to the surface of the soil. Each treatment group of 12 tubes were then closed with cellulose acetate stoppers and kept in groups according to treatment type in a lidded plastic container lined with moist cloth to create a humid atmosphere. Boxes were kept at 17-20° C. on the laboratory bench (with natural and fluorescent lighting during the day) unless otherwise specified. Cabbage was changed, and live slugs were counted weekly. Each week over a 4 week challenge slugs were inspected and scored for mortality and food was replaced.

### Tub Experiments

1 kg soil (horticultural sandy loam, pH 6.9) was used in 25 L plastic tubs with lids. The soil depth was ˜5 cm. Groups of slugs (1-1.5 cm) were placed in 500 ml plastic boxes lined with damp filter paper and 2×10⁶ trophonts cultured either in PPYE or MYE were poured onto the filter paper. The boxes were closed for 24 hours and then opened and placed in the 25 L tubs lined with sandy loam. The tubs were kept at 17-20° C., 12 hour light. Cabbage discs 6.5 cm diameter were added as food. Uneaten food was photographed and new food discs provided weekly. The proportion of the food that was eaten was estimated, giving a good indication of grazing. At the end of the experiment the tubs were slowly flooded and surviving slugs picked off the surface of the soil and counted.

### Gel Encapsulation of Pre-Formed Encysted Ciliate Cells in Alginate

T. rostrata cysts were made using the soil infusion buffer method at 26° C. for 24 hours resulting in 100% encystment. The cysts were collected by centrifugation (800 g, 10 min) resuspended in soil infusion and then mixed with 1.2% alginate at a vol:vol ratio of 1:4. The cyst/alginate suspension was loaded into a syringe pump and extruded at the rate of 3 ml per minute; dropping into a 50 mM CaCl₂ bath on a magnetic stirrer to form cross-linked alginate hydrogel beads. The beads were washed twice in water after 5 min gelation. Alginate beads were stored at 20° C.

### In-Gel Encystment of Trophont Ciliate Cells in Alginate

T. rostrata trophonts were harvested from culture by centrifugation (800 rpm, 10 min) and either resuspended in PPYE at ˜10⁵ cells/ml or were washed in 10 mM HEPES pH7 and resuspended at 10⁵ cells/ml, and then mixed with 1.2% alginate at a vol:vol ratio of 1:4. The trophont/alginate suspension was loaded into a syringe pump and extruded at the rate of 3 ml per minute; dropping into a 50 mM CaCl₂ bath on a magnetic stirrer to form cross-linked alginate hydrogel beads. The beads were washed twice in water after 5 min gelation. Alginate beads were stored at 20° C.

### Viability of Alginate Encapsulated Ciliate Cells

Alginate beads were stored in sealed tubes at 20° C. in the dark and periodically samples were dissolved using sodium citrate to assess the morphology and viability of the encapsulated cells. The concentrations of sodium citrate used were selected after developing procedures to release cells which either retained their morphology or alternatively, were viable and could grow.

For morphology assessment, beads were each dissolved in 200 μl of 12.5 mM sodium citrate buffer for 2 hr at 20° C. and dispersed by gentle pipetting. Samples from each of three beads were mounted on slides under coverslips and examined at ×40 magnification. All cells in the field of view were determined as being round, cyst-like cells in contrast to rostrate cells.

The number of viable encapsulated beads was assessed by dissolving beads in 200 μl 3.125 mM sodium citrate buffer for 16-24 hours. The viability of the released cells was determined using a Most Probable Number procedure (MPN) in 96 well microtiter trays using PPYE media incubated at 20° C. for 6-8 days. The ability of each dilution to establish a culture was scored as growth or no growth and the MPN of viable cells per bead was calculated according to Jarvis et al. (2010). The basis of the MPN method is to serially dilute a sample until the inoculant will sometimes but not always contain one or more viable organisms. Multiple wells are inoculated with each dilution and the result is the number of wells with growth which will imply the number of viable cells in the original, undiluted sample. The MPN is the number which makes the observed outcome most probable and the 95 percent confidence intervals bracket the range of numbers for which there is at least a 95% chance that the range includes the actual concentration.

### Suspension of Trophont Ciliate Cells in Carboxymethylcellulose (CMC) Hydrogel

Trophonts were harvested from culture by centrifugation (800 rpm, 10 min) and either resuspended in PPYE at ˜10⁵ cells/ml or were washed in 10 mM HEPES pH7 and resuspended in PPYE at 10⁵ cells/ml.

Trophont cultures were incorporated into a physical crosslinked carboxymethylcellulose (CMC) hydrogel. In this example, trophont cultures were incorporated into the CMC hydrogel by mixing 1 part of PPYE cell culture (4×10⁵ cells/mL) with 3 parts of a sterile 1.5% CMC (w/v in water) solution containing 0.5% CaCl₂. The starting density of the CMC cell suspension was 1×10⁵ cells/mL.

The CMC hydrogels were then stored at 4° C. or 20° C. in PPYE, and each week for 4 weeks, samples from gels and controls (no CMC hydrogel) were removed to test cell viability and cell morphology (observation by microscopy and subculturing into fresh media and observing the results by microscopy).

### Gel Encapsulation of Trophont Ciliate Cells in Carboxymethylcellulose (CMC)-Alginate Core-Shell Hydrogel Beads

Core-shell hydrogel beads comprising an outer alginate shell with liquid CMC centres comprising trophonts were made using a trophont suspension (9.1×104 cells/ml) mixed in a 1:3 ratio with sterile 1.5% w/v CMC solution containing 1.5% w/v CaCl₂.2H₂O (34 mM 5CaCl₂.2H₂O). The mixture was dropped into a sterile 0.875% w/v alginate solution. Beads were removed from the alginate and washed in water. Some beads were then hardened briefly in 0.9% w/v CaCl₂.2H₂0 (61 mM) to further cross-link the alginate shell. Three hardened and 3 unhardened beads were placed in 500 μl of 0.5% PPYE or 10 mM HEPES pH7 in a 12 well tissue culture plate and incubated at 20° C. for a week to assess the survival and multiplication of the cells within the CMC core of the beads. Cells were viewed under ×40 magnification and photographed after one week.

Release of Ciliate Cells from within Hydrogels and Culturing

For cell morphology and viability studies, hydrogels encapsulating or suspending ciliate cells were incubated in PPYE, water or sodium citrate buffer to release ciliate cells.

Nuclear Staining of Cells Released from Hydrogels

Giemsa stain is used to stain the various stages of the T. rostrata nuclei. Attempts to use the Giemsa stain directly on sections of bead were not successful since the cells did not adhere to the coverslip and got washed away during the staining process. Therefore, the cells had to be harvested in buffer before fixing them on coverslips. In brief, 0.5 mL of 10 mM sodium phosphate buffer (the same buffer used to make up the stain) was added to 2-3 beads in a 15 mL centrifuge tube. The beads were ruptured using a sterile fine blade scalpel and vortexed to mix. They were then centrifuged at 500 g for 5 mins, and the tube was left undisturbed for 10-15 mins until some cells (released from beads) swim up to the supernatant.

The objective was to harvest the released cells (theronts excysted from cysts) excluding any alginate gel residue. The supernatant was used to make smears on coverslips and was air-dried at 27° C. for 1-2 hrs. After this, the Giemsa stain procedure was followed.

### Example 2—Isolation and Identification of T. rostrata TRAUS

The examination of cells cultured from T. rostrata TRAUS under bright field and scanning electron microscopy (SEM) confirmed that cells were ovoid with a rounded posterior end and narrower anterior end. The ellipsoid buccal aperture which was situated near the anterior end of the cell and a caudal cilium was observed in some cells. The measurements derived from SEM images showed trophonts 34-39×23-26 μm covered in somatic cilia arranged in 24-26 longitudinal rows. The buccal aperture had distinct ciliary membranelles. In some specimens, where the cilia had been sheared off, six parallel rows of membranelles within the grooved buccal cavity could be seen.

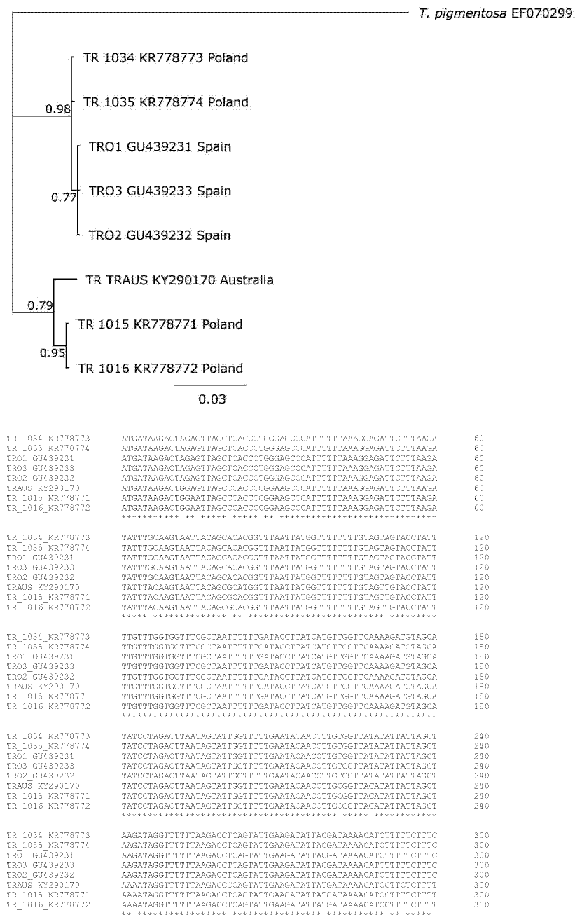

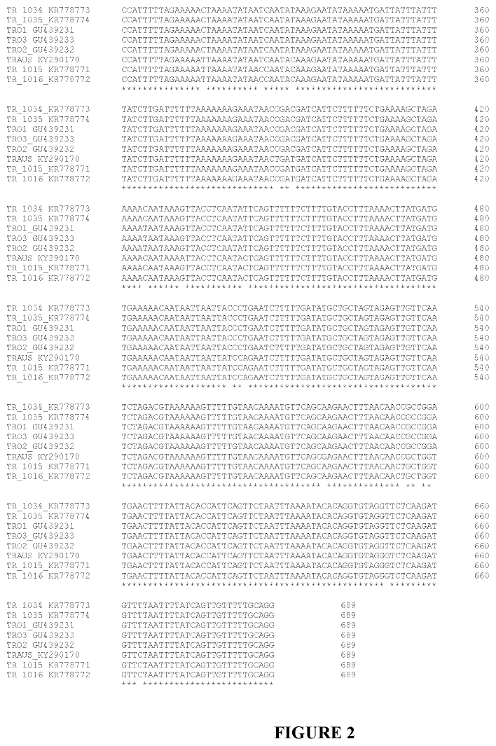

T. rostrata TRAUS cox1 (SEQ ID NO:7) is 98.7% identical to TR 1016 and TR 1015 and 95.7-95.8% identical to TRO1, TRO2, TRO3, TR 1035 and TR 1034 indicating that they are all the same species (FIG. 2).

The isolated strain of T. rostrata TRAUS comprises a mitochondrial DNA sequence as shown in SEQ ID NO:1.

### Example 3—Encystment in Tris Buffer or Soil Infusion Buffer (SI-H)

T. rostrata TRAUS trophonts suspended in Tris buffer, 26° C. behaved in a similar manner as the strains used by Kaczanowski et al. (2016). Pre-cystic, fast swimming tomites were observed, cells rounded and cysts were formed. Three days after the starvation stimulus was applied, 50% of the cells were cysts and the rest were motile. However, in the hands of the inventors, large numbers of the trophonts lysed shortly after being placed in the Tris buffer and we did not continue these experiments further.

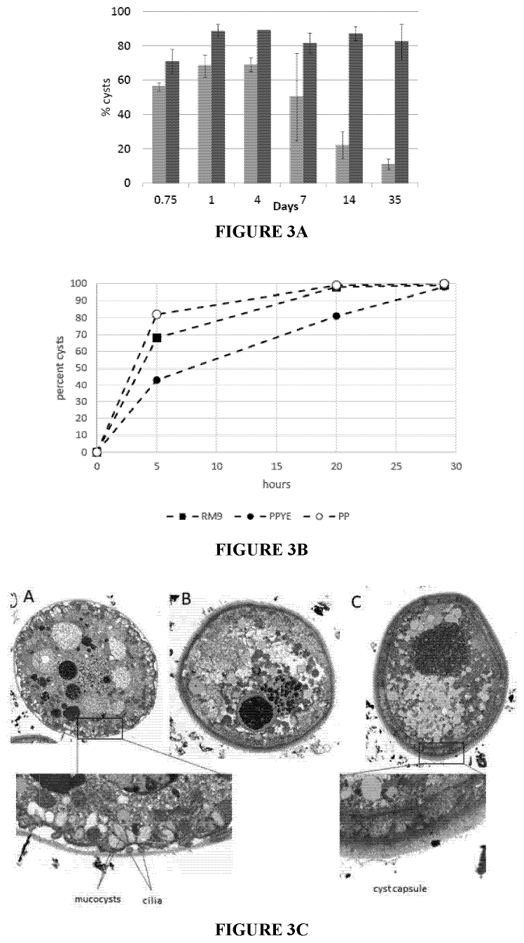

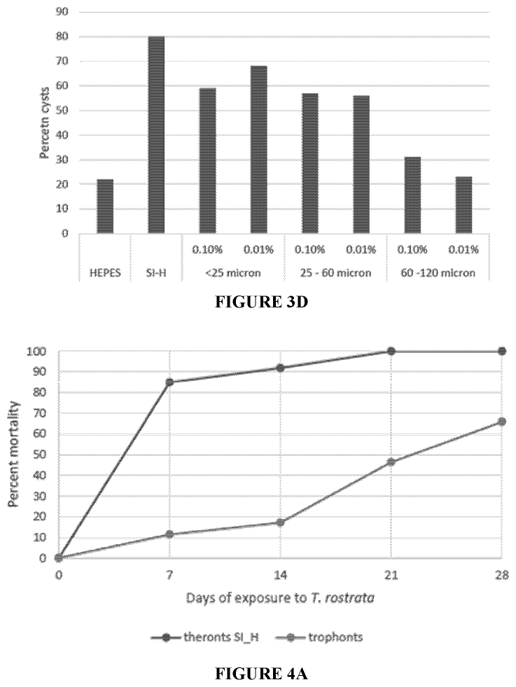

Encystment in soil infusion buffer at 20° C. and 26° C. were performed. At 20° C., encystment peaked after 24 hours when 60-90% of the cells were cysts (FIG. 3A, light grey) and the remainder were free swimming. The cells that were not cysts at 24 hours were mainly trophonts. Spontaneous excystment was observed after seven days and this continued, so by 35 days only 10% of the cells were cysts (FIG. 3A, light grey). At 26° C., 84-93% of the cells were cysts after 24 hours and no spontaneous excystment occurred during 35 days of observations unless the temperature was reduced (FIG. 3A, dark grey). Up to 96-100% encystment of trophonts to cysts have also been achieved. If the preparations were moved to 20° C. then 70% of the cells excysted within 24 hours even without addition of any nutrients. Segade et al. (2016) also noted that changes in temperature could result in spontaneous excystment in starvation medium. In their work, using strain TRO1, cells encysted in mineral water at 30° C. could excyst when moved to 18° C. In our hands, soil infusion used at 30° C. killed most of the cells of T. rostrata TRAUS and no encystment occurred in mineral water. The durability of the cysts formed at 26° C. was similar to Segade et al. (2016) who reported a sharp decrease in viability after 28 days at 30° C.

Cysts that formed in soil infusion buffer were reproductive cysts going through autogamy, as evidenced by their nuclear arrangements. Reproductive cysts which formed in soil infusion buffer at 26° C. and were then incubated at 20° C. took 5 to 7 days to complete autogamy, resulting in cells with the characteristic two macronuclear units and one (or two) micronuclei. Spontaneous excystment occurred at the completion of autogamy at 20° C. The data suggest that encystment was synchronised and excystment was coordinated. As discussed above, excystment was inhibited at 26° C. and the cells completed autogamy and developed into resting cysts. Cysts made using soil infusion buffer encystment were fixed and sectioned for examination by transmission electron microscopy. Cells showed mucocysts discharging and developing the cyst capsule (FIG. 3C).

These experiments provided a method to prepare cultures that were purely theronts and also highlighted that encystment of ciliate cells in soil infusion or buffer methods were delicate and excysted into theront cells at 20° C.

### Effect of Culture Media on Encystment in Soil Infusion Buffer

The ability of trophonts grown in RM9, PPE and PP to form cysts in soil infusion buffer at 26° C. over 6 days was investigated. Almost all cells grown in RM9 and PP encysted within 20 hours whereas cells grown in PPYE took longer to encyst and were fully encysted by 29 hours (FIG. 3B). The morphology of cysts was confirmed using Giemsa staining. It was found that culturing in PPYE provided the best yield of trophonts while pre-culturing in PP resulted in both a higher % of cysts after 5 hours and more durable cysts following encystment in soil infusion buffer. Based on this, PPYE was used as the culture medium for bulk culturing and PP was used as the pre-culture medium for encystment in soil infusion buffer.

### Effect of Age of Trophont Culture on Encystment in Soil Infusion Buffer

Encystment is required periodically for T. rostrata to maintain high levels of viability and infectivity. The culture ‘ages’ if the micronuclei are not renewed by autogamy. The number of passages (i.e. subcultures) in culture give an indication of the time elapsed since cells last went through autogamy. When an ‘aged’ culture (13 passages, approximately 117 generations) and a “young” culture (2 passages, approximately 18 generations) were encysted in soil infusion buffer, the efficiency was 80-86% for the older culture and 99-100% for the younger culture. The data demonstrates that optimal encystment over a two week period is obtained using a culture that has passaged less than 10 times before encysting. Theronts released when either young aoraged cultures are encysted and then excysted killed slugs.

### Effect of Soil Particle Size on Encystment in Soil Infusion Buffer

The potting soil used to make the soil infusion buffer was composed of medium grade compost pine bark, wetting agent (Saturaid™), sand and trace elements supplement (Elemax, Australian growing solutions). A HEPES buffered infusion of composted pine bark without the other additives was as effective for triggering encystment as a soil infusion buffer indicating that the wetting agent and other soluble components of the potting mix did not contribute to encystment.

Preliminary experiments demonstrated that soil particles <25 μm aided in the development of cyst aggregates. Soil sieves were used to separate small particles from milled pine bark into sizes <25 μm, 25-60 m and 60-120 m and the particles were suspended 10 mM HEPES buffer at either 0.1 or 0.01% w/v. Soil infusion buffer and HEPES buffer without particles were used for comparisons in encystment done at 20° C. The percent of cysts in each mixture was assessed after 24 hours. The largest number of cysts formed in soil infusion buffer and the least cysts formed in HEPES buffer alone and in suspensions of larger particles, 60-120 m (FIG. 3D).

### Example 4—Infection of Adult D. reticulatum with Trophonts of T. rostrata TRO1 and TRAUS

Trophonts of T. rostrata TRO1 and TRAUS were compared in a D. reticulatum bioassay to determine their pathogenicity. Mortality and egg laying was monitored. By the end of the experiment on day 42, 89% of the TRAUS exposed slugs had died as compared with 60% for TRO1 and 24% of the untreated controls (FIG. 24). The result showed that trophonts of T. rostrata TRO1 were pathogenic for D. reticulatum, something that had not been previously demonstrated. Furthermore, the newly isolated T. rostrata TRAUS was also capable of killing slugs.

Eggs laid in the first week, when no adults had died, were incubated until hatching occurred and the survival of neonates was assessed. Only 3.6% of all eggs laid by slugs challenged with TRAUS and 15.8% of those treated with TRO1 had neonates which survived 6 weeks after the egg laying date. This contrasted with a survival rate of 54.1% for neonates from the control slugs. A small number of eggs laid by slugs treated with TRAUS or TRO1 had ciliates in the egg sac, 40/2228 (1.8%) and 30/3903 (0.8%) respectively and were confirmed by sequencing to be the treatment strains. This demonstrated transovarial transmission and suggested that young slugs might be particularly sensitive to T. rostrata.

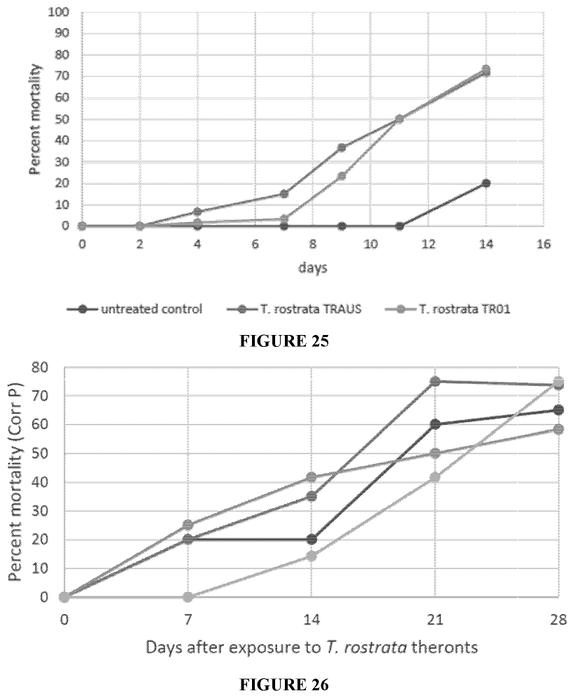

The low survival of neonates was investigated further using newly hatched D. reticulatum. Newly hatched slugs were collected within 48 hours of hatching and challenged with T. rostrata TRAUS or TRO1 trophonts. Slugs died rapidly and by day 9, there were significantly more deaths among the TRO1 and TRAUS treated slugs treated slugs compared to deaths in the controls (FIG. 25). There was a similar death rate of TRAUS and TRO1 slugs through to day 14 of the assay. These results showed that young and newly hatched slugs were sensitive to T. rostrata and died more rapidly than adults.

Theronts are More Effective at Killing D. reticulatum than Trophonts

All previous reports of infection studies using laboratory grown T. rostrata have used the readily cultured trophont developmental stage. A distantly related tetrahymenid ciliate, Ichthyopthirius multifiliis is an ectoparasite of freshwater fish. Although I. multifiliis has trophonts (also known as trophozoites) and cyst life stages; it is the newly excysted cells which are the infective form. Similarly another tertahymenid, Lambornella clarki go through encystment to infect their host mosquito larvae. This raised the question whether excysted theronts of T. rostrata might be more effective at killing D. reticulatum than trophonts.

The previous experiments also showed that trophonts could kill slugs. The other various developmental stages of T. rostrata were investigated to see if infectivity differed. Accordingly, we compared trophonts and theronts of T. rostrata TRAUS in a challenge experiment.

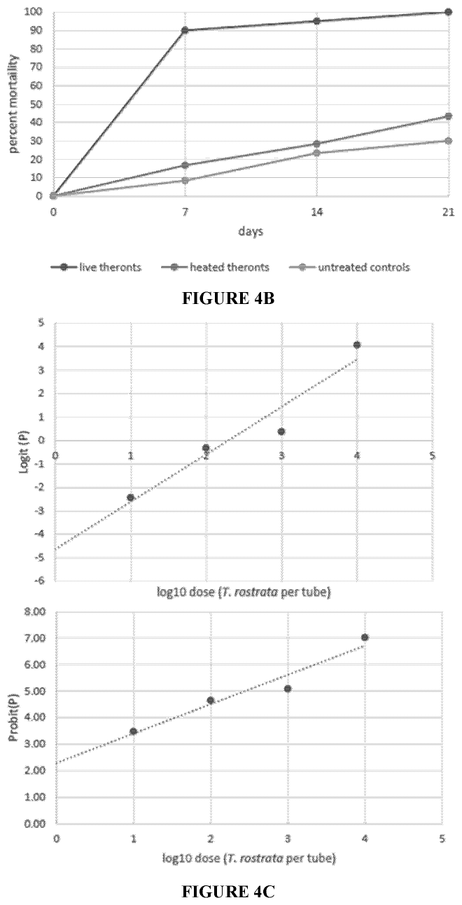

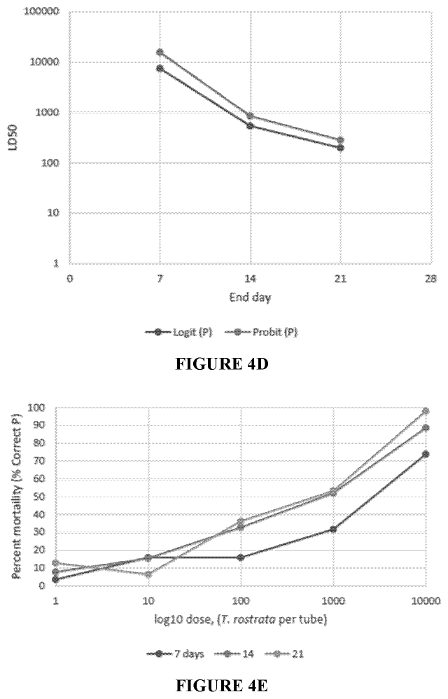

The growth, encystment and excystment conditions for T. rostrata were manipulated to produce trophonts and theronts. These were been used in experiments where young D. reticulatum slugs were exposed in tubes to 1.4-3×10⁴ T. rostrata cells. Mortality in the first seven days were higher (FIG. 4A) with theronts (released from SI-H cysts) than with trophonts. These results indicate that theronts are the most infective form. Theronts killed slugs faster than trophonts.

An experiment was performed using live and heat-killed theronts to assess whether the mortality was due to an infection or an intoxication. In this instance, half of the preparation of theronts were killed in a 65° C. water bath for 10 minutes and the other half were kept at room temperature. Fifteen replicate tubes of 5 slugs per tube were exposed to no theronts, 2×10⁴ heat killed theronts and 2×10⁴ to unheated theronts and mortality was assessed over 21 days. There were few deaths from heat killed theronts and high levels of mortality among slugs exposed to live theronts which indicates infection as the cause of the deaths (FIG. 4B).

A dose/response experiment was performed with D. reticulatum and T. rostrata theronts which had been prepared by encystment in soil infusion buffer at 26° C. and then excystment at 20° C. There were four slugs per tube and 15 replicate tubes per treatment. The doses per tube were 0, 1 10, 100, 1000 and 10,000 theronts. The experiment was conducted at room temperature (17-20° C.) for 21 days and the number of live slugs was assessed every seven days. Mortality was corrected for deaths in the control groups and Logit and Probit analysis show good correlation of the mortality data. The LD₅₀ is indicated by the 0 intercept of the Logit plot (FIG. 4C top) and the 5 intercept of the Probit (P) plot (FIG. 4C bottom).

Fifty percent of slugs were killed by seven days using 7,433-15,471 theronts per tube, by 2 weeks using 536-281 theronts per tube, and by three weeks using 196-281 theronts per tube (FIG. 4D). Mortality was delayed when using the lower doses and this was probably because of the time course for the amplification of the dose through the multiplication of T. rostrata within the slugs. The Corrected Proportion dead vs dose vs times shows most death occurs within the first 7 days when using doses 100 to 10,000 T. rostrata per tube (FIG. 4E).

The effect of temperature on slug mortality was investigated with more deaths of slugs exposed to T. rostrata theronts at 12° C., 16° C. and 20° C. temperatures compared with slugs that were not exposed to T. rostrata theronts. Slugs died at all temperatures, but mortality was faster at 20° C.

The effect of T. rostrata theronts on Ambigolimax valentianus, and Limacus flavus slugs was also investigated. The results showed that both A. valentianus and L. flavus were also susceptible to T. rostrata theronts (see FIG. 26).

The feeding behaviour of slugs exposed to T. rostrata theronts was investigated as to whether there was evidence of reduced grazing owing to infection. The feeding behaviour of slugs (D. reticulatum and A. valentianus) strongly reduced in the first 7 days following exposure to theronts.

### Example 5—Encapsulation of Pre-Formed Cysts in Hydrogels Stabilises them for Long Term Storage at 20° C.

T. rostrata TRAUS cells encysted using the soil infusion buffer method at 26° C. were encapsulated in alginate hydrogel beads and stored at 20° C. The cysts encapsulated in the alginate hydrogel remained distributed throughout the hydrogel (FIG. 6A). Cysts do not have cilia and are not motile. The fact that the cysts remain dispersed confirms the response of trophonts migrating to the core of alginate hydrogels is a biological chemotactic response rather than any passive diffusion (see Example 8).

The number of cysts encapsulated per bead was determined by releasing the cysts from the bead by immersion in sodium citrate buffer and then cysts were counted showing there were approximately 1000 cells/bead. The ability of the cysts to excyst after release from the bead was determined using MPNs.

After 59 days at 20° C. all of the cells were still encysted cells and 69.2% were able to subsequently excyst and establish new populations in culture. Encystment ability has been demonstrated even after 68 days. This result is in contrast to the cysts that were made in soil infusion without alginate which spontaneously excysted at temperatures below 26° C. and lost viability.

In another experiment, the stability and viability of cysts after encapsulation in hydrogel beads was assessed. In the first experiment, cysts made in soil infusion buffer or MgSO₄ buffer (see Example 8) at 26° C. for 24 hours and were encapsulated in alginate with ˜200 cells/bead. Encystment using soil infusion buffer is more efficient than in magnesium sulfate buffer with the proportions of cysts formed being 98±2% and 76±6% respectively. This result showed that resting cysts made in soil infusion buffer could be stabilised and kept encysted if encapsulated in alginate. While fewer cysts produced with MgSO₄ buffer survived encapsulation, viable cysts were present after 30 days. There were also some excysted cells apparent and the overall yield was not as high as for the soil infusion buffer encapsulated cysts. Overall, cysts were maintained over several weeks at room temperature without excysting.

The encapsulation of soil infusion buffer cysts was repeated using 211 cells/bead (99-100% cysts) and MPNs were done on cells released after 30, 55 and 68 days (Table 1). The results indicated that all the cells remained encysted and there was no apparent loss of viability between 30 and 68 days.

TABLE 1
Viability and stability of soil infusion buffer cysts encapsulated
in alginate for up to 68 days at 20 Celsius

| | Day 0 | Day 30 | Day 55 | Day 68 |
|---|---|---|---|---|
| Cells/bead^{a} | 925 | — | — | — |
| Proportion of cysts after | 99-100% | — | — | — |
| encystment at 26° C., | | | | |
| 24 hr^{a} | | | | |
| Viable count^{b} | — | 720 | 640 | 760 |
| 95% CI-high^{d} | — | 320 | 310 | 430 |
| 95% CI-low^{d} | — | 1300 | 1300 | 1300 |
| Proportion (%) of round, | — | 100 | 100 | 100 |
| cyst-like cells^{c} | | | | |

^{a}2 microscopic counts.
^{b}1 MPN on 4 beads.
^{c}3 microscopic counts.
^{d}MPN 95% confidence intervals.

### Example 6—Trophont Ciliate Cells Suspended in CMC Hydrogel Remain Viable

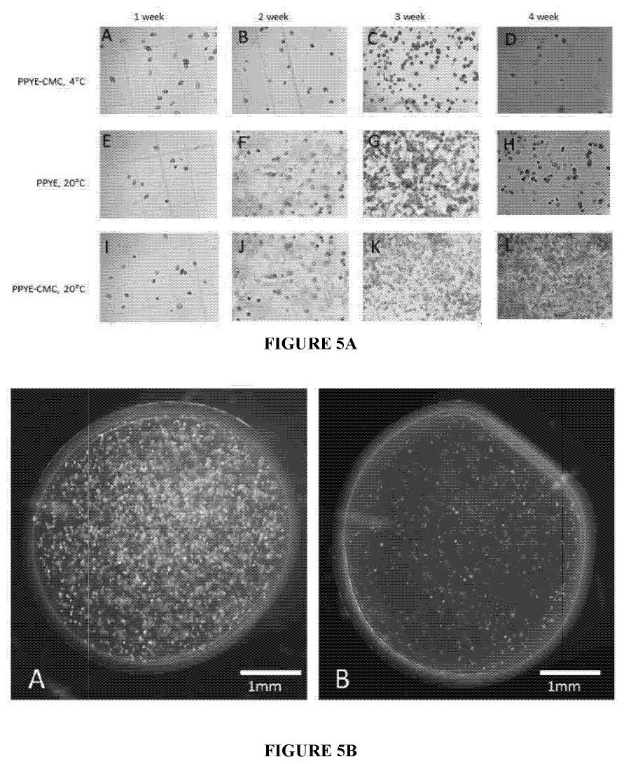

Trophont ciliate cells were evenly distributed and suspended in the CMC hydrogel and did not settle to the bottom of the vessel or migrate to the surface (FIG. 5A, a) and b)). At 4° C., the growth of the PPYE-CMC ciliate cells were slower and cells retained their trophont shape (FIG. 5A, c) and d)). At 20° C., the PPYE-CMC ciliate cells had multiplied and entered stationary phase after 4 weeks (FIG. 5A, i)-l)). Cells in media alone (no CMC suspension) at 20° C. had also multiplied and entered stationary phase after 4 weeks (FIG. 5A, e)-h)).

Cells in PPYE-CMC multiplied when they were incubated at 20° C. (FIG. 5A, i)-l)). These results showed that T. rostrata trophonts could tolerate encapsulation in CMC and that they will remain viable and even multiply in CMC if the temperature and nutrient supply is suitable.

The subculturing showed that the ciliate cells suspended in CMC were viable and more readily multiplied in fresh media compared to the control cells in media. In contrast to the alginate hydrogel, the ciliate cells suspended within the CMC hydrogel remained as trophont ciliate cells and survived at 4° C. for 1 month. Slugs ate the CMC hydrogel.

In contrast to alginate hydrogels, the CMC hydrogel did not encapsulate the ciliate cells but rather formed a liquid hydrogel which could flow but was still capable of suspending the ciliate cells.

Encapsulation of Trophont Cells Suspended in CMC Hydrogel with an Outer Alginate Shell to Form CMC-Alginate Core-Shell Beads

The CMC hydrogels comprising suspended ciliate cells were functionalised with an alginate shell to make core-shell hydrogel beads that that had a viscous CMC core and an alginate shell. These core-shell hydrogel beads have a permeable alginate shell where nutrients can diffuse into the CMC core and metabolites can diffuse out of the central CMC core. Such functionalisation with an alginate outer shell can include one or more attractant or feeding stimulants to encourage slugs to graze on the alginate shell thus rupturing and releasing T. rostrata from the CMC cores. One or more shells can be added, wherein each shell could comprise a different attractant/feeding stimulant.

Here, trophonts suspended in CMC hydrogel were encased in alginate shells to create core-shell hydrogel beads. One set of core-shell beads were hardened in a CaCl₂ bath to further cross-link the alginate shell and the other set of core-shell beads were not further hardened. It will be appreciated that the further hardening results in an alginate shell with different physical properties to the unhardened shell.

CMC-alginate core-shell hydrogel beads containing trophonts were incubated in nutrients (PPYE) or without nutrients (10 mM HEPES pH7) at 20° C. for a week and then inspected using an inverted microscope to determine if the cells survived, multiplied or encysted. The cells multiplied in both the hardened and in unhardened spheres incubated in PPYE and as expected, and they did not multiply in spheres incubated in buffer only (FIG. 5B). In both types of spheres, motile cells could be observed swimming through the CMC cores. Encystment could be triggered via diffusion of starvation media through the porous alginate shell.

### Example 7—Magnesium Sulfate Induces Encystment of T. rostrata

In some cases, soil infusion is not a practical buffer for reproducible encystment. If the soil was left to infuse for too long then encystment failed. Chemical analysis of preparation with different infusion times revealed the significant variable was the concentration of MgSO₄. A buffer was prepared with an optimal MgSO₄ concentration and it was used to encyst T. rostrata TRAUS at 20° C. and 26° C.

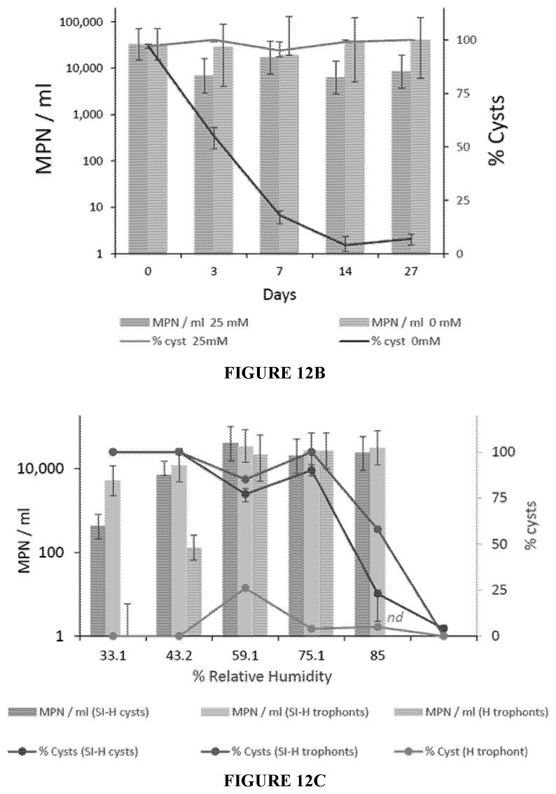

It was observed that after three days 96% to 99% of trophont cells encysted when incubated at 26° C. in 10 mM HEPES pH 7 with MgSO₄ concentrations ranging from 62.5 to 125 μM, and 55% to 65% of trophont cells encysted when incubated at 20° C. in 10 mM HEPES pH 7 with MgSO₄ concentrations ranging from 125 to 250 μm (FIG. 12A). Cells excysted when transferred to nutrient medium. The optimum conditions for this encystment involved suspending trophonts cells in 62.5 M MgSO₄/10 mM HEPES followed by incubation at 26° C. for 48 hours. As a result, MgSO₄ was identified as a trigger for encystment of T. rostrata.

### Magnesium Sulfate Stabilises Encysted Ciliate Cells

Other methods of cyst stabilisation were explored. Cysts were prepared using soil infusion buffer at 26° C. for 24 hours and then suspended in different concentrations of MgSO₄ in 10 mM HEPES pH7 and kept at 20° C. for 27 days. Cells excysted when the MgSO₄ concentration was less than 12.5 mM. However they remained encysted and viable in 25-50 mM MgSO₄-10 mM HEPES.

The results for the cysts in 0 and 25 mM MgSO₄-10 mM HEPES are shown in FIG. 12B. Cysts treated with HEPES buffer without MgSO₄ excysted, so, although there are high MPN values, the proportion of cysts was very low after 7 days. In contrast, cysts treated with 25 mM MgSO₄ remained viable and a high proportion remained in cyst-form for the duration of the experiment.

### Tolerance of Encysted Ciliate Cells to Dehydration

Cysts were prepared in soil infusion buffer (SI-H) at 26° C. for 24 hours and were placed in closed containers at 20° C. suspended in humidity chambers above different saturated salts which created a range of relative humidity. The dehydrated cyst suspensions viability was tested by MPN assays.

Briefly, cysts were prepared in soil infusion buffer at 26° C. for 24 hours and were placed in closed containers at 20° C. suspended in humidity chambers above different saturated salts which created a range of relative humidity (ranging from 0 to 97.6% relative humidity). After 18 days in 50 mm dishes, the culture was resuspended to its original volume and MPN per ml were determined and plotted. Starting culture was 1×10⁴ encysted cells/mL. Cysts incubated at 43.2-75.7% relative humidity remained viable for 18 days and remained encysted. This result demonstrated that encysted ciliate cells could be treated and stabilised at 20° C. without the need for encapsulation. The relative number of cysts (% round) and the viable count (MPN) are shown in FIG. 12C. The results showed the cysts can be dehydrated to a certain level and under those conditions the cysts remain stable (i.e. do not spontaneously excyst).

### Dehydration of Trophonts at Room Temperature Produces Stable Cysts

Trophonts were resuspended in soil infusion buffer or HEPES buffer in closed containers at 20° C. suspended in humidity chambers above different saturated salts which created a range of relative humidity (ranging from 0 to 97.6% relative humidity). After 18 days in 50 mm dishes, the culture was resuspended to their original volume and MPN per ml were determined and plotted. Starting culture was 2.8×10⁴ and 4.7×10⁴ cells/ml for soil infusion buffer trophonts and HEPES buffer trophonts, respectively.

It was discovered that dehydration could trigger encystment of trophonts to form stable cysts at room temperature. In particular, trophonts in soil infusion buffer were encysted at 43.2-75.7% relative humidity and the cysts remained viable (FIG. 12C). In contrast, trophonts in HEPES buffer alone did not encyst when dehydrated. This demonstrated that dehydration of trophonts can trigger encystment in soil infusion buffer to form cysts and the formed cysts remain stable (i.e. did not spontaneously excyst) at room temperature when under dehydration conditions. This relates to the water activity of the cyst

### Example 8—T. rostrata Undergo Encystment within Hydrogels and Cysts Remain Stable

Surprisingly, it was observed that the trophonts migrated to the centre of the alginate bead during gelation (FIG. 6A). Cross-linking of the alginate hydrogel is initiated by the diffusion of Ca²⁺ cations into the alginate matrix while the bead is suspending in the cross-linking bath. Trophonts were harvested from culture as usual, washed in 10 mM HEPES, and were mixed with 1.5% w/v alginate solution in a 4:1 v/v ratio. Droplets were dropped from the syringe pump into a stirred 50 mM CaCl₂ bath and allowed to gel for 5 minutes and then they were washed in water. Beads were stored at 20° C. All of the cells in the gel beads migrated to the core and some formed cysts. Active trophonts could be seen within the aqueous interstitial voids within the hydrogel and could be released upon physical crushing or dissolution of the hydrogels.

MPN assays on 21-day old beads showed over 50% of the cysts could encyst under the conditions used. Microscopic examination of 83-day old beads show that all of the cells were still cysts and they could be stimulated to start moving around inside the cyst coat which indicates they are viable and capable of excystment (FIG. 9). The alginate beads produced measured approximately 3 mm in diameter, spherical with a pointed tip. The centre where the ciliate cells are concentrated is visible (FIG. 6B).

The migration is believed to be due to trophont cell aversion to the high density of Ca²⁺ cross-linker at the surface of the hydrogel and/or the mechanical effect of forming the hydrogel. In other words, the migration of trophonts during crosslinking appears to be a chemotactic response to Ca²⁺ (or Cl⁻) ions present from the cross-linker and/or to the mechanical effect of gelling of the alginate. Interestingly, this migration phenomenon was not observed within the CMC cores of the CMC-alginate core-shell beads, even with additional hardening, confirming the stimulus for the chemotactic response is therefore likely to be due to the mechanical formation of the gel. Studies have reported that the high polyvalent cross-linker concentrations (such as 50 mM Ca²⁺) retards the encystment process, however the reduced level of cross-linker of Ca²⁺ within the bead would be favourable for the encystment. In addition, due to this migration, there is an increase in cell density at the centre of the hydrogel, and this overcrowding, coupled with the exposure to Ca²⁺ cations, causes the encystment of the trophont cells into encysted ciliate cells.

### Example 9—Shelf Life Studies

Light microscope images and videos of the encapsulated cells were recorded each week for stored alginate beads. Videos were important records for the ‘activity’ of cells, which reduced with the storage time. Videos were critical especially in the case of cysts, as the clock-wise and anti-clockwise rotations depicted the dynamic nature of the cysts. However, it should be noted that these images were not snap-shots of encapsulated cells inside the cyst, rather images of released cells immediately after opening a bead. There were no significant differences in morphology in weeks 2-3, while weeks 1 and 4 showed noticeable differences especially in movement. The dimensions of the cysts in alginate beads were comparable with literature, (Kaczanowski et al., 2016), which reported cysts with a mean length of 58 m, and a mean width of 24 m, and that it was less than half of the trophont size, which are similar to our observations.

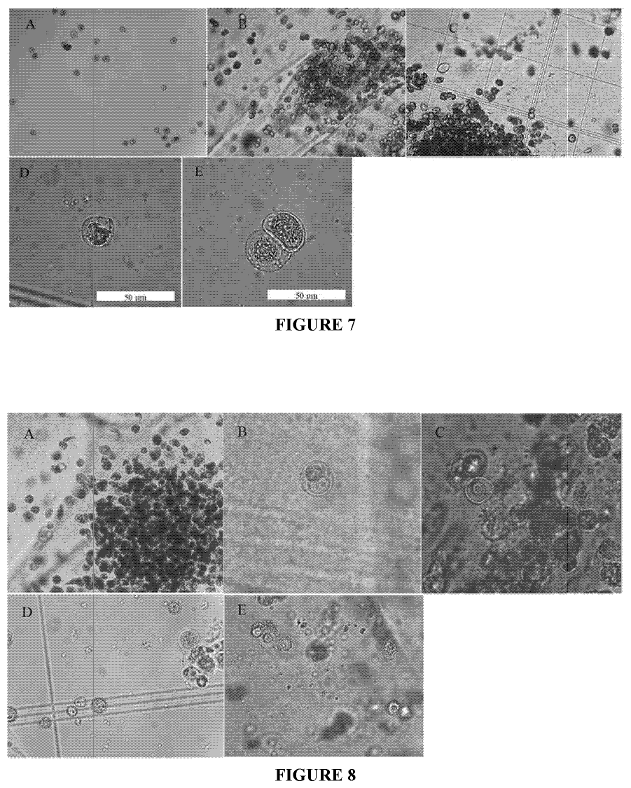

Week 1: In week one the cells in the centre were still very active. Some had the trophont shape immediately after releasing from the bead (FIGS. 7B and 7C). There were some ‘round’ cells which were similar to stationary phase cells (see FIG. 7A) in a normal culture, and some cysts with a large gap (or filled with cyst wall material) between the cell and the outer cyst membrane (FIGS. 7D and 7E). Cysts were rotating fast inside this space. The inventors assume this is a gap filled with some excreted material rather than a thick wall as the gap was changing with the cell movement.

Week 4: At week 4, the encysted cells were not moving but were in a resting state within the cysts wall. Cells were viable and could be stimulated to move and excyst (see FIG. 8).

### Example 10—Nuclear Staining Shows Encapsulated Cells Excyst into Theront Cells when Released from Hydrogel

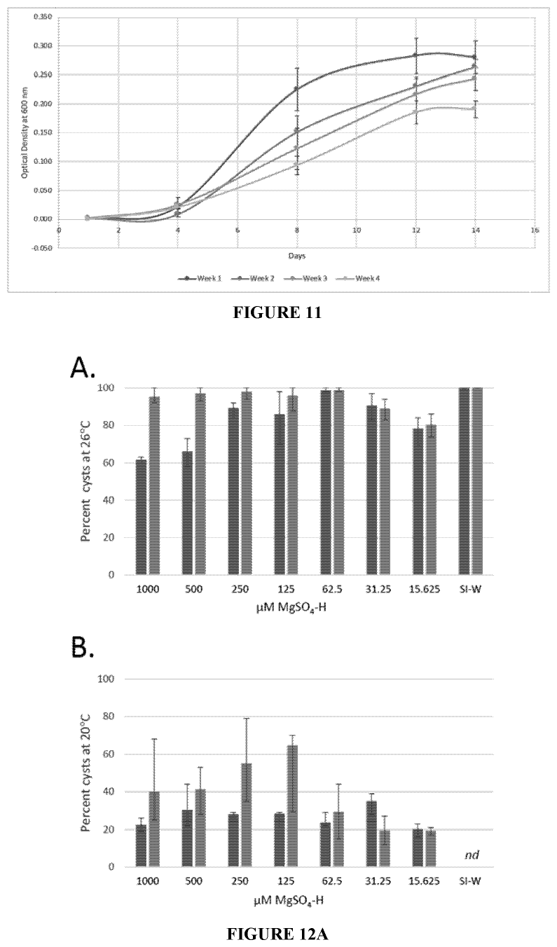

FIG. 10 shows stained cells harvested from 4 week-old beads (stored in sterile 50 mL tubes, with minimum amount of Milli-Q water, at 20° C.). The stained nuclei showed the characteristic butterfly effect, as compared to the defined macro and micro nuclei in a trophont. Only rarely, (FIG. 10D, top right of the image) were such characteristic trophont nuclei still observed. These results prove that cells immediately released from beads are theronts and provide further evidence that the microscopy images recorded of the encapsulated cells are of viable encysted cysts.

### Example 11—Growth Curves for Alginate Encapsulated Ciliate Cells after Storage for 1-4 Weeks

Alginate beads can be dissolved by displacing the physically cross-linked Ca²⁺. Cells were released from gel beads using either 12.5 mM sodium citrate buffer, water or PPYE media. The number of cells capable of excystment was determined using the most probable number (MPN) method. Each sample was diluted 2-fold in PPYE and incubated at 20° C. The MPNs were using 4 replicates.

The growth of semi-continuous cultures was monitored using optical density (OD) measurements. A standard curve was first established for growth of a standard culture, correlating OD measurements with hematocytometer cell counts. For semi-continuous culturing, 1 alginate bead was inoculated in 20 mL PPYE, in triplicate. From each culture triplicate 250 μL were transferred to round-bottom 96 well plate, and OD was measured at 600 nm. OD measurements were continued until cultures reached stationery phase, after which, the OD measurements were not accurate due to cell debris accumulating. Ciliate cells encapsulated within alginate hydrogels demonstrated characteristic growth highlighting good viability during storage (see FIG. 11). Alginate hydrogels dissolved with 12.5 mM sodium citrate gives rise to cells retaining their original forms (cysts or trophonts). Cells released with lower sodium citrate concentrations (3.125 mM sodium citrate) remained viable and multiplied and were used for viability counts in MPN assays.

### Example 12—Summary of Encapsulation and Encystment Experiments

The T. rostrata TRAUS strain is lethal for the D. reticulatum slugs and it has been demonstrated that they can encyst using several methods. Theront cells have also been identified as being highly infective to slugs.

Cells stimulated to encyst using a 24-hour incubation in soil infusion at 26° C. could be encapsulated in alginate beads and stored at 20° C. for more than two months and still could excyst. Trophonts encapsulated in alginate beads migrated to the core of the bead and encysted and could be stored at 20° C. and remained viable for at least 83 days. These formulations are suitable for applying to soil where gels will release cysts into the environment. The hydrogel approach for utilisation of T. rostrata and other trophont and/or encysted ciliate cells for biological control of pest molluscs represents an advancement in the field.

### Example 13: Confirmation of Infection of T. rostrata on D. reticulatum

The pathogenic effects of T. rostrata on D. reticulatum were assessed though controlled exposure. Slugs were assessed for changes in behaviour as well as histopathological impacts of infection. In regard to behaviour changes, slugs were observed for impacts on locomotion, response to adverse stimuli, swollen or hunched appearance and movement of tentacles. D. reticulatum has two pairs of tentacles, superior and inferior, both are mechanosensory and olfactory organs whereas only the superior tentacles have eyes. Slugs were found to have impaired movement of the superior tentacles as a result of exposure to T. rostrata. The severity of superior tentacle impairment was graded as mild moderate and severe.

Confirmation of infection was achieved though histological examination of exposed slugs. Slugs exposed to T. rostrata were found to have ciliates present predominantly in their renal tissue, with some found in the heart, muscle and interstitial spaces. Ciliates found in the renal tissue were larger than those seen in the muscles and interstitial spaces and were dividing. A small number of slugs were also found to have tumours as the result of exposure (FIG. 23). This study confirmed active infection of D. reticulatum by T. rostrata.

### Cultures

Cultures of T. rostrata were maintained at 20° C. in the dark in a medium of 0.25% Protease Peptone (Oxoid, LP0085), 0.25% yeast extract (Oxoid, LP0021) and 0.125% glucose (w/v) (PPYE), subcultures were performed fortnightly. Cultures prepared for encystment were prepared in 0.25% Protease Peptone, and 0.125% glucose (w/v) (PP).

### Preparation of Theront Inoculum

Experiment 1: T. rostrata was cultured in PP for 7 days to mid log phase and pelleted at 800×g 10 min. Cells were washed in 10 mM HEPES pH 7. Cells were suspended in a buffered aqueous soil solution comprising composted pine bark particles (referred to as CI) buffered with 10 mM HEPES pH 7 at a final concentration of 1×10⁴.

Cells were then plated into 6 well plates (Greiner bio-one Cat no. 657 185) in 3 mL aliquots. Plates were incubated at 26° C. for 24 hours and then moved to 20° C. for 6 days. Cells were then resuspended, transferred to a 50 mL tube and centrifuged at 300×g. The cells were then left undisturbed for 2 hours. Without disturbing the cell pellet the supernatant was removed and a cell count performed on the theronts.

Experiment 2 and 3: Theronts were prepared as above with the exception that cells were encysted in a buffered aqueous solution comprising soil soil infusion containing fine bark particles (referred to as SI) buffered with 10 mM HEPES pH 7.

### Selection of Slugs for Tests

Slugs are maintained in as described herein. Slugs are removed from home boxes and placed on a 1 cm×1 cm grid and allowed to move around. 1 cm Slugs were selected. Slug size is judged by the fully stretched length of the slug. Any slug that appeared to have a reduction in fitness was not selected for the experiment.

### Housing of Animals During Experiments

Slugs were individually housed in small round containers, 5 cm tall tapered 5.5-6.5 cm wide. Cabbage (˜2 cm²) was added to each container. To ensure the environment did not dry out, containers were placed 10 to a tub lined with two Chux® damp with distilled H₂O.

### Inoculation of Slugs

Inoculum was pipetted onto the slugs and cabbage. Control slugs were exposed to a mock inoculum of the same volume of buffered solution of either 10 mM HEPES pH 7 buffered soil infusion comprising composted soil particles (CI) or soil infusion containing particles (SI).

Experiment 1: 400 μl of 2.1×10⁴ CI theronts;

Experiment 2: 300 μl of 3.0×10⁴ SI theronts; and

Experiment 3: 300 μl of 3.1×10⁴ SI theronts.

### Monitoring of Slugs

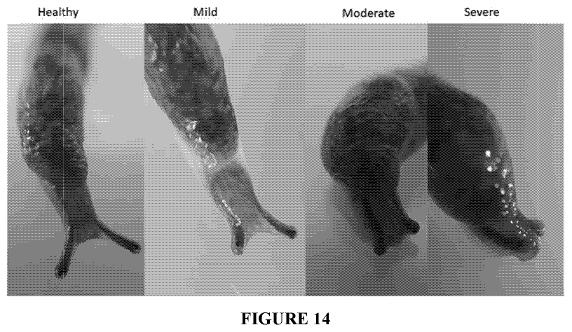

Slugs were monitored for signs of ill health. This was recorded as 0, 1, 2 or 3 for superior tentacle mobility. Superior tentacle mobility assesses the slug's ability to move their ocular tentacles. Healthy slugs immediately retract and extend ocular tentacles in response to stimuli scoring a 0. Slugs experiencing ocular mobility difficulties cannot extend their tentacles, slow extension scored a 1 (mild) and little extension scored a 2 (moderate) while no extension scored a 3 (severe). FIG. 14 provides an example of the monitoring of the superior tentacle mobility.

### Histology Protocol

After 7 days of exposure 10 slugs from the control group and 30 slugs displaying superior tentacle impairment from the theront exposed group were selected and euthanised. Of the theront exposed slugs 10 were selected displaying mild, 10 moderate and 10 severe superior tentacle impairment. Slugs were euthanised by submersing in soda water. Once slugs had stopped moving for at least 5 minutes they were transferred to neutral buffered formalin. After 2 days they were transferred to 70% ethanol. The fixed samples were processed, embedded in paraffin wax and sectioned transversely into 5 μm thick sections. These were mounted on glass slides and stained with haematoxylin and eosin (H & E). The slides were then examined using a Leica DMLS light microscope.

### Statistical Analysis

Statistical analysis was performed using GraphPad Prism version 8.3.0. Significance of survival was assessed using survival curves and log-rank test and odds ratio. The presence of ciliates in histological samples was assessed using Fisher's exact test and the Chi-squared test.

### Mortality and Histology Results

A summary of results for each experiment and the encystment solution used to prepare theronts is presented in Table 3.

TABLE 3
Summary of conditions for all slug exposure experiments.
Compost infusion (CI), soil infusion (SI).

| | | | | | Ocular |
|---|---|---|---|---|---|
| | | Theront | Volume | Mortality | impairment |
| Exp # | Solution | Concentration | inoc | at 7 days | onset (days) |
| 1 | CI | 2.1 × 10⁴ | 400 μl | 58.3% | 1 |
| 2 | SI | 3.0 × 10⁴ | 300 μl | 41.6% | 1 |
| 3 | SI | 3.1 × 10⁴ | 300 μl | 75% | 1 |

### Mortality

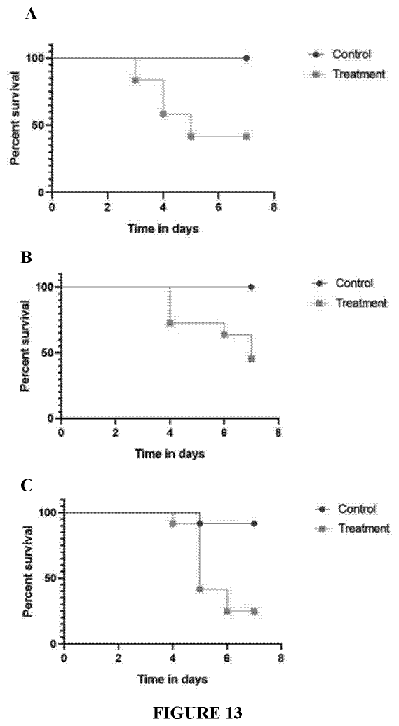

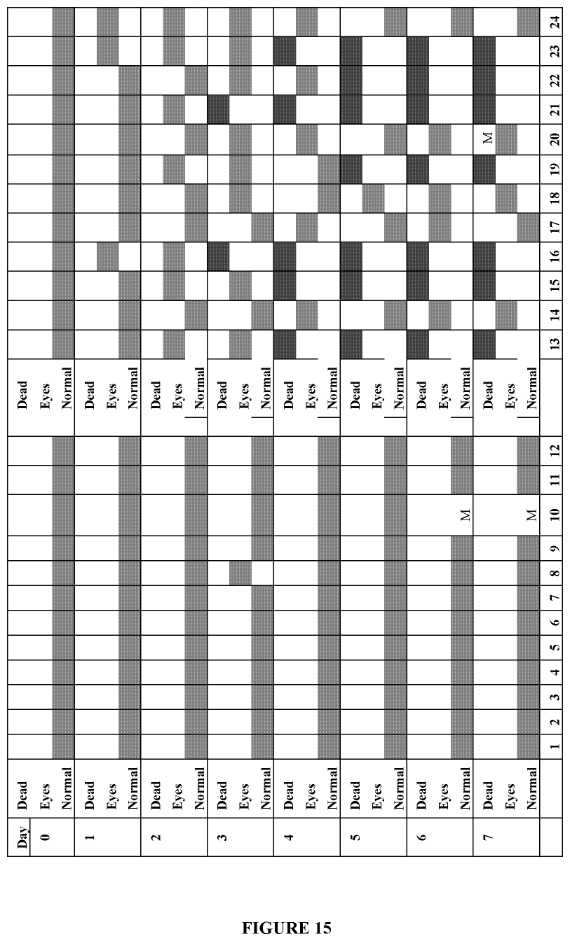

Experiment 1: Slugs exposed to theronts showed a significantly higher mortality rate than the control group with a 58.3% mortality after 7 days of exposure (P<0.002) (FIG. 13A). Slugs exposed to theronts rapidly showed signs of superior tentacle impairment as compared to the control group (FIG. 15). 25% of exposed slugs displayed signs after 1 day of exposure and 58.3% after 2 days of exposure. Slugs began to die after 3 days of exposure (FIG. 15).

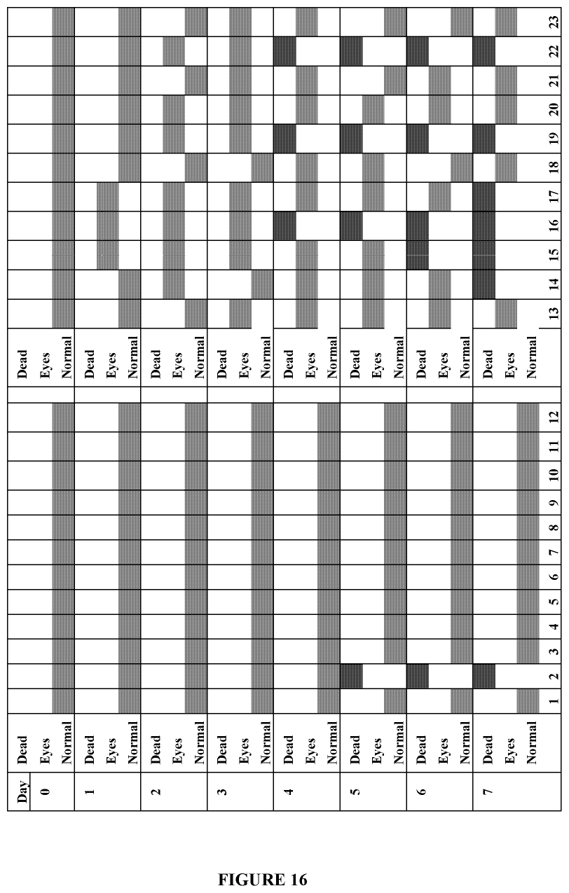

Experiment 2: Slugs exposed to theronts showed a significantly higher mortality rate than the control group with a 50% mortality after 7 days of exposure (P<0.0033) (FIG. 13B). Slugs exposed to theronts rapidly showed signs of superior tentacle impairment as compared to the control group (FIG. 16). 25% of exposed slugs displayed signs after 1 day of exposure and 58.3% after 2 days of exposure, slugs began to die after 4 days of exposure.

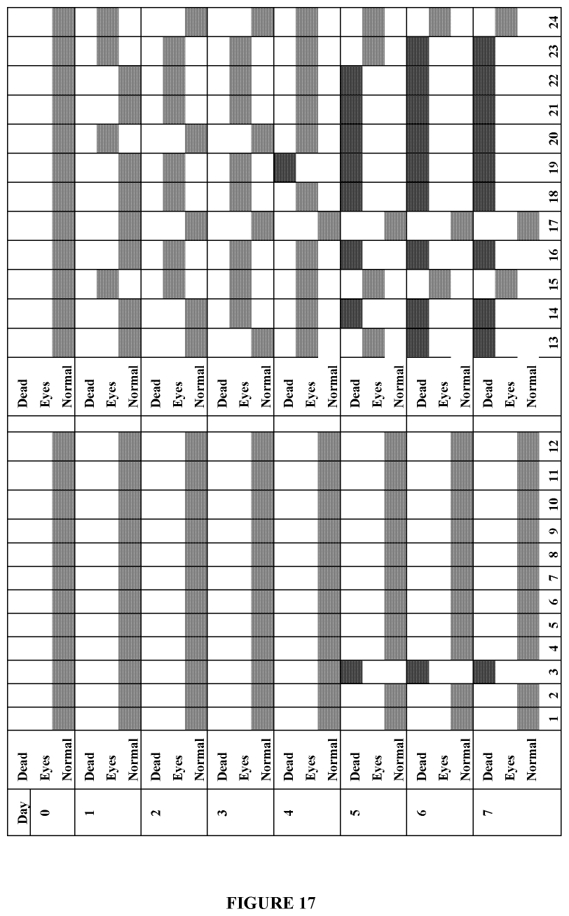

Experiment 3: Slugs exposed to theronts showed a significantly higher mortality rate than the control group with 75% death rate after 7 days of exposure (P<0.0011) (FIG. 13C). Slugs exposed to theronts rapidly showed signs of superior tentacle impairment as compared to the control group (FIG. 17). 33.3% of slugs displayed signs after 1 day of exposure and 58.3% after 2 days, slugs began to die 4 days after exposure.

Results from Experiments 1, 2 and 3 were compiled and odds ratio was performed. Odds ratio was used to assess the likelihood of death and impairment of superior tentacles as a result of exposure to theronts. Slugs exposed to theronts were 27.92 times more likely to die (95% CI 5.94 to 126.7) (P<0.0001) and 1225 times more likely to display superior tentacle impairment (95% CI 78.22 to 12284) (P<0.0001) than control slugs.

### Histology

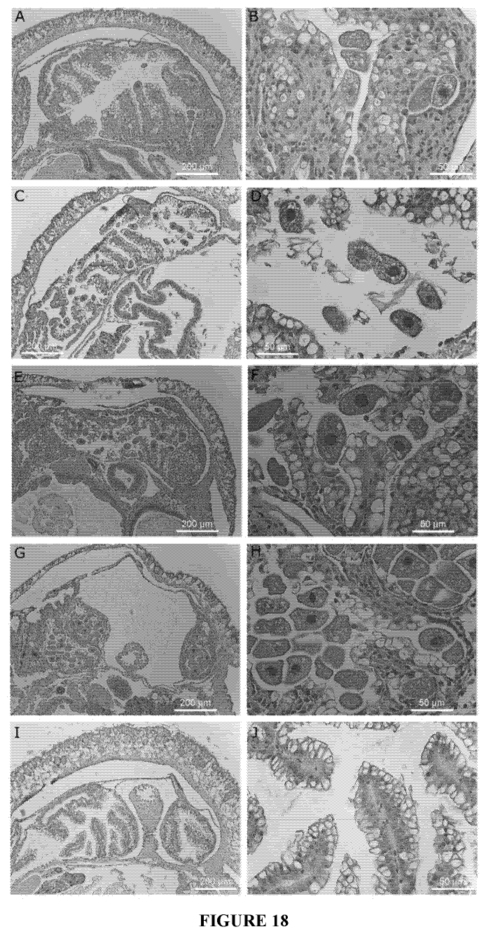

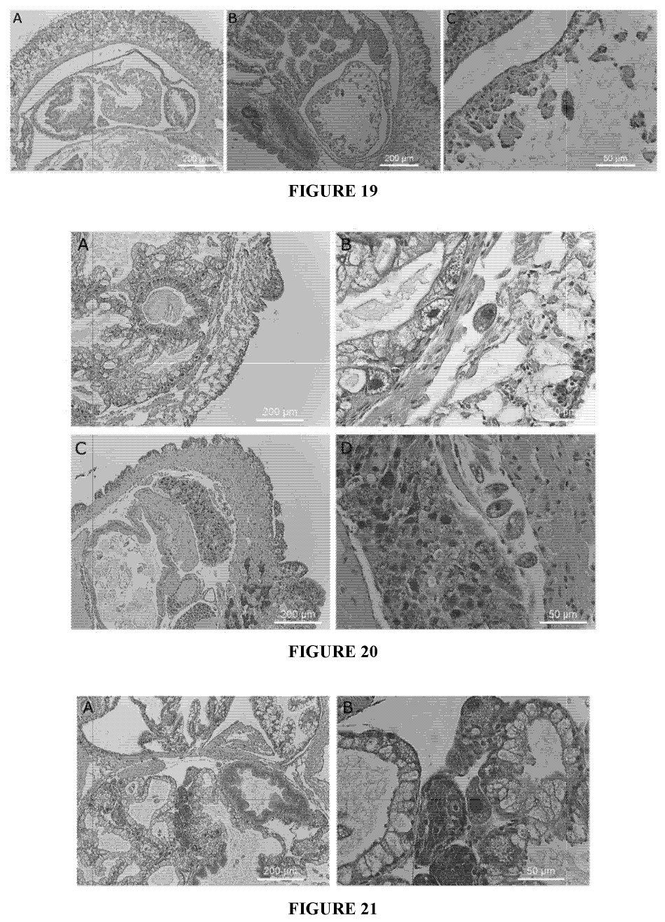

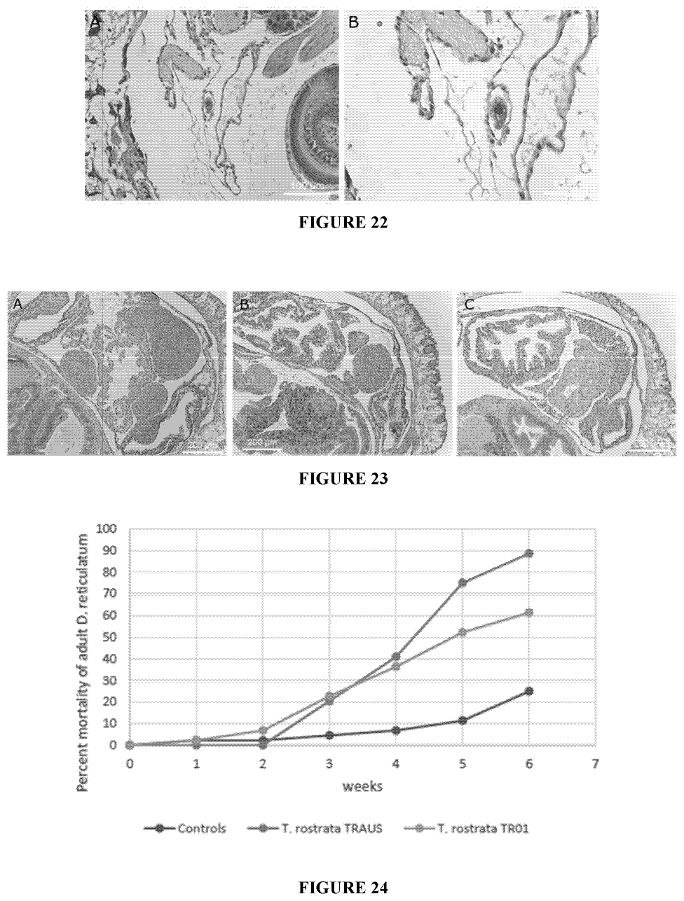

The presence of ciliates observed in histological sections was analysed using a contingency table and Fisher's exact test. Slugs exposed to theronts are significantly more likely to have ciliates in their body (P<0.0001). Ciliates were found in the tissues slugs exposed to theronts. Ciliates were most commonly found in the renal tissue (FIG. 18). Ciliates were found in the renal tissue (FIG. 18), heart (FIG. 19), muscle (FIG. 20) interstitial space (FIG. 21) arteries (FIG. 22) hepatopancreas and pneumostome passage. Tumour structures were also identified in three of the exposed slugs (FIG. 23).

### Discussion of Mortality and Histology Results

In Experiment 1, the slugs were monitored every day for seven days only, recording the effect of theronts on superior tentacle mobility and death. The results showed that slugs rapidly died (FIG. 13A) and that they began to show signs of impaired superior tentacle movement within 1 day of exposure (FIG. 15). This experiment demonstrated that superior tentacle impairment is a result of exposure to theronts and precedes death in exposed slugs.

The results of Experiments 2 and 3 closely mirror those of Experiment 1. Slugs begin to show superior tentacle impairment after 1 day of exposure and begin to die after 4 days. Experiments 1, 2 and 3 are replicates of each other with the only difference between them being the formation of cysts in CI buffer for Experiment 1 and SI for Experiments 2 and 3. When comparing these three experiments, the overall mortality rate is 58.3%. The compiled results of these experiments confirm that slugs are 27.92 times more likely to die as a result of exposure to theronts (P<0.0001) and that exposure to theronts results in 1225 times more likely to develop impairment of superior tentacles (P<0.0001).

Comparing the mortality rate from these Experiments show that theronts cause mortality in D. reticulatum. In particular, the onset of superior tentacle impairment preceded death of the theront exposed slugs.

The histological results show that after exposure theronts can be seen within the tissues of the slugs. Ciliates were commonly found in the renal tissue (FIG. 18). The ciliates found in the renal tissue, between the skin and muscle, and the interstitial space are larger than those seen in the gut and have an identifiable micronucleus marking them as T. rostrata. The ciliates found in the renal tissue appeared to be multiplying, as dividing cells can be seen (FIG. 18D). The ciliates are very large and their motility within the slug is highlighted by the presence of ciliates as individuals in places other than the renal tissue. Ciliates in the renal tissues favour the saccular portion. They cause damage to the renal cells leaving the basal cells intact. The mechanism for T. rostrata in the destruction of the renal tissue was enzymatic, mechanical or both highlighted by the ciliates apparent grazing on the renal cells (FIG. 18F).

Ciliates are seen in the muscle, between the skin and muscle layers of the slug and in the interstitial space (FIG. 20). A ciliate was identified in an artery (FIG. 22). The ciliates can travel from the renal tissue to the heart and circulatory system. The ciliates in the muscle appear smaller than those in the renal tissue (FIG. 20). The ciliates are also found individually beside the developing gonads in the interstitial space a few slugs (FIG. 21)

Slugs exposed to theronts and examined though histology displayed abnormal changes in the heart. In FIG. 23 the pericardial cavity of these three slugs has filled with tumours and hypertrophic amoebocytes. These tumours originate from the wall of the heart and renal tissue. In FIG. 23 c the pericardial cavity has aggregating masses of hypertrophic amoebocytes. The results shown confirm that T. rostrata is pathogenic to slugs and colonisation is mainly seen in the renal tissue. This result is further strengthened as the experimental infections here have been performed on colony reared slugs, which reduces the impact of unknown pathogens.

The histological sectioning also provides insight into the life stage of the ciliates inside the slugs. Slugs were exposed to theronts, the newly excysted form of T. rostrata. This life form has a characteristic lobulated macronucleus. The invading theronts convert after feeding to trophonts. The ciliates that were seen in the sectioned slugs show the characteristic form of trophonts with the single round macronucleus. Infection with the theront form of T. rostrata results in death of exposed slugs from both theront and trophont damage. The primary routes of infection are likely to be through the pneumostome or mantle pouch.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU 2019903410 filed 13 Sep. 2019, the entire contents of which are incorporated herein by reference.

### REFERENCES

Carrick (1939) Transactions of the Royal Society of Edinburgh 59(3):563-597
Jarvis et al. (2010) J. Appl. Microbiol. 109:1660-1667
Kaczanowski et al. (2016) Protist 167:490-510
Katoh et al. (2013) Molecular Biology and Evolution 30:772-780
Keasre et al. (2012) Bioinformatics 28:1647-1649
Needleman and Wunsch (1970) J. Mol Biol. 45:443-453
Parhi et al. (2017) Adv Pharm Bull 7:515-530
Segade et al. (2016) Parasitol Res 115:771-777

1. A composition comprising a hydrogel and a population of ciliate cells, wherein the ciliate cells are encapsulated or suspended within the hydrogel, wherein the hydrogel comprises a physically cross-linked hydrogel-forming polymer.

2. The composition according to claim 1, wherein the ciliate cells are encysted ciliate cells or trophont ciliate cells.

3. The composition according to claim 1 or claim 2, wherein the ciliate cells are encysted ciliate cells.

4. The composition according to claim 1 or claim 2, wherein the ciliate cells are trophont ciliate cells.

5. The composition according to claim any one of claims 1 to 4, wherein the hydrogel comprises about 0.1% w/v to about 5% w/v of the hydrogel-forming polymer.

6. The composition according to any one of claims 1 to 5, wherein the hydrogel-forming polymer is a polysaccharide.

7. The composition according to any one of claims 1 to 6, wherein the hydrogel-forming polymer is selected from one or more of alginate, cellulose, gellan gum, starch, chitin, chitosan, hyaluronan, or carboxymethylcellulose (CMC).

8. The composition according to any one of claims 1 to 7, wherein the hydrogel-forming polymer is alginate or carboxymethylcellulose (CMC).

9. The composition according to any one of claims 1 to 8, wherein the hydrogel-forming polymer is alginate.

10. The composition according to any one of claims 7 to 9, wherein the alginate is sodium alginate.

11. The composition according to any one of claims 1 to 10, wherein the hydrogel-forming polymer is ionically cross-linked.

12. The composition according to claim 11, wherein the hydrogel-forming polymer is ionically cross-linked by a polyvalent cation.

13. The composition according to any one of claim 12, wherein the polyvalent cation is a divalent cation or a trivalent cation, or a mixture thereof.

14. The composition according to claim 13, wherein the divalent cation or trivalent cation is selected from one or more of Ca²⁺, Mg²⁺, Sr²⁺, Ba²⁺, Zn²⁺, Be²⁺, Fe³⁺, Al³⁺ or Mn³⁺.

15. The composition according to claim 13 or claim 14, wherein the divalent cation is Ca²⁺.

16. The composition according to any one of claims 1 to 15, further comprising magnesium sulfate.

17. The composition according to any one of claims 1 to 16, wherein the hydrogel comprises a plurality of hydrogel beads, wherein one or more of the hydrogel beads encapsulates one or more of the ciliate cells.

18. The composition according to claim 17, wherein the hydrogel beads have an average size of about 100 μm to about 5 mm in diameter.

19. The composition according to any one of claims 1 to 18, wherein the hydrogel further comprises an attractant or feeding stimulant.

20. The composition according to claim 19, wherein the attractant is a nutrient source or a pheromone.

21. The composition according to claim 19 or claim 20, wherein the attractant is provided as an outer coating on the hydrogel.

22. The composition according to any one of claims 17 to 21, wherein the average number of ciliate cells encapsulated in the one or more hydrogel beads is about 100 to about 10,000 ciliate cells per bead.

23. The composition according to claim 22, wherein the average number of ciliate cells encapsulated in the one or more hydrogel beads is about 1000 ciliate cells per bead.

24. The composition according to any one of claims 1 to 23, wherein the ciliate cells encapsulated or suspended in the hydrogel remain viable for at least about four weeks.

25. The composition according to any one of claims 1 to 24, wherein the ciliate cells are any member of the Ciliophora phylum.

26. The composition according to any one of claims 1 to 25, wherein the ciliate cells are a member of the Heterotrichea, Karyorelictea, Armophorea, Litostomatea, Colpodea, Nassophorea, Phyllopharyngea, Prostomatea, Plagiopylea, Oligohymenophorea, Protocruziea, Spirotrichea, or Cariotrichea class.

27. The composition according to any one of claims 1 to 26, wherein the ciliate cells are a member of the Apostomatia, Astomatia, Hymenostomatia, Peniculia, Peritrichia, or Scuticociliatia order.

28. The composition according to any one of claims 1 to 27, wherein the ciliate cells are a member of the Tetrahymenidae, Ophryoglenina, or Peniculina family.

29. The composition according to any one of claims 1 to 28, wherein the ciliate cells are a member of the Tetrahymena genus.

30. The composition according to any one of claims 1 to 29, wherein the ciliate cells are of the T. rostrata, T. hegewischi, T. hyperangularis, T. malaccensis, T. patula, T. pigmentosa, T. pyriformis, T. thermophila, T. vorax, T. geleii, T. corlissi, T. empidokyrea or T. limacis species.

31. The composition according to any one of claims 1 to 30, wherein the ciliate cells are of the T. rostrata species.

32. A method of encapsulating or suspending a population of ciliate cells within a hydrogel, the method comprising:
a) adding a suspension of ciliate cells to a hydrogel-forming polymer solution to form a hydrogel,
wherein the ciliate cells are encapsulated or suspended within the hydrogel.

33. The method according to claim 32, wherein step a) comprises adding a suspension of ciliate cells to a hydrogel-forming polymer solution and an ionic cross-linker solution to form a hydrogel, wherein the ciliate cells are encapsulated or suspended by the hydrogel.

34. The method according to claim 32 or claim 33, wherein the ciliate cells in step a) are trophont ciliate cells.

35. The method according to claim 34, wherein the trophont ciliate cells are encapsulated by the hydrogel and undergo encystment within the hydrogel to form one or more encysted ciliate cells.

36. The method according to claim 32 or claim 33, wherein the ciliate cells in step a) are pre-formed encysted ciliate cells.

37. The method according to any one of claims 33 to 36, comprising:
a1) preparing a mixture comprising the suspension of ciliate cells and the hydrogel-forming polymer solution and adding the mixture of a1) to the cross-linker solution to form the hydrogel.

38. The method according to claim 37, wherein one or more droplets of the mixture of step a1) are added to the cross-linker cation solution to form the hydrogel.

39. The method according to any one of claims 32 to 38, wherein step a) or step a1) further comprises magnesium sulfate.

40. The method according to claim 39, wherein the concentration of the magnesium sulfate is about 20 μM to about 100 μM.

41. The method according to any one of claims 33 to 40, wherein the suspension of ciliate cells and the hydrogel-forming polymer solution is exposed to the cross-linker solution for less than about 20 minutes.

42. The method according to any one of claims 33 to 41, wherein the suspension of ciliate cells and the hydrogel-forming polymer solution is exposed to the cross-linker solution for about 1 minute to about 10 minutes.

43. The method according to any one of claims 33 to 42, wherein the suspension of ciliate cells and the hydrogel-forming polymer solution is exposed to the cross-linker solution for about 5 minutes.

44. The method according to any one of claims 32 to 43, wherein the density of ciliate cells in the suspension of ciliate cells is about 1×10⁵ cells/mL.

45. The method according to any one of claims 32 to 44, wherein the hydrogel-forming polymer in the hydrogel-forming polymer solution has a concentration of about 0.1% w/v to about 5% w/v.

46. The method according to any one of claims 32 to 45, wherein the hydrogel-forming polymer in the hydrogel-forming polymer solution has a concentration of about 1.5% w/v.

47. The method according to any one of claims 32 to 46, wherein the vol:vol ratio of the suspension of ciliate cells to the hydrogel-forming polymer solution is about 1:4.

48. The method according to any one of claims 32 to 47, wherein the hydrogel-forming polymer solution comprises a polysaccharide.

49. The method according to any one of claims 32 to 48, wherein the hydrogel-forming polymer solution comprises one or more of alginate, cellulose, gellan gum, starch, chitosan, chitin, hyaluronan or carboxymethylcellulose (CMC).

50. The method according to any one of claims 32 to 49, wherein the hydrogel-forming polymer solution comprises alginate or carboxymethylcellulose (CMC).

51. The method according to any one of claims 32 to 50, wherein the hydrogel-forming polymer solution comprises alginate.

52. The method according to claim 51, wherein the alginate is sodium alginate.

53. The method according to any one of claims 33 to 52, wherein the cross-linker solution comprises polyvalent cations.

54. The method according to claim 53, wherein the concentration of the polyvalent cations in the cross-linker solution is about 20 mM to about 500 mM.

55. The method according to claim 53 or claim 54, wherein the concentration of the polyvalent cations in the cross-linker solution is about 50 mM.

56. The method according to any one of claims 53 to 55, wherein the polyvalent cations in the cross-linker solution are divalent cations or trivalent cations, or a mixture thereof.

57. The method according to claim 56, wherein the divalent cations or trivalent cations are selected from one or more of Ca²⁺, Mg²⁺, Sr²⁺, Ba²⁺, Zn²⁺, Be²⁺, Fe³⁺, Al³⁺, or Mn³⁺.

58. The method according to claim 56 or claim 57, wherein the divalent cations are Ca²⁺.

59. The method according to any one of claims 33 to 58, wherein the cross-linker solution is calcium chloride (CaCl₂).

60. The method according to any one of claims 32 to 59, wherein the hydrogel is in the form of a plurality of hydrogel beads.

61. The method according to any one of claims 32 to 60, wherein the ciliate cells are located in the centre of the hydrogel beads.

62. The method according to any one of claims 32 to 61, wherein the hydrogel beads have an average size of about 100 μm to about 5 mm in diameter.

63. The method according to any one of claims 33 to 62, further comprising the step b) washing the formed hydrogel to remove any excess cross-linker solution.

64. The method according to claim 63, further comprising the step c) storing the washed hydrogel in a sealed container.

65. The method according to claim 64, wherein the hydrogel is stored in the dark.

66. The method according to claim 64 or claim 65, wherein the hydrogel is stored at about 4° C. to about 28° C.

67. A method of inducing the encystment of ciliate cells, the method comprising incubating a population of trophont ciliate cells in a buffer solution comprising magnesium ions, wherein the trophont ciliate cells undergo encystment to form one or more encysted ciliate cells.

68. The method according to claim 67, wherein the buffer solution comprises magnesium sulfate.

69. The method according to claim 67 or 68, wherein the trophont ciliate cells are incubated in the buffer solution at a temperature of about 20 to 30° C.

70. The method according to any one of claims 67 to 69, wherein the trophont ciliate cells are incubated in the buffer solution for about 12 to 48 hours.

71. The method according to any one of claims 68 to 70, wherein the concentration of magnesium ions in the buffer solution is about 15 μM to about 500 μM.

72. An isolated strain of T. rostrata which has one or more or all of the following features:
i) deposited under PTA-126056 on 13 Aug. 2019 at the American Type Culture Collection,
ii) comprises a mitochondrial genome which has a nucleotide sequence as shown in SEQ ID NO:1 or a sequence at least 90% identical thereto, and
iii) comprises a cox1 gene which has a nucleotide sequence as shown in SEQ ID NO:7 or a sequence at least 99% identical thereto.

73. A composition comprising the T. rostrata strain of claim 72, and one or more acceptable carriers.

74. A method of infecting or colonising a pest species with a ciliate, the method comprising applying to an area affected or likely to be affected by a pest species one or more of a hydrogel composition according to any one of claims 1 to 31, a hydrogel composition or encysted ciliate cells prepared by the method according to any one of claims 32 to 71, a strain of T. rostrata of claim 72 or the composition of claim 73.

75. The method according to claim 74, comprising adding the hydrogel with a solution to disrupt the ionic cross-linking in the hydrogel prior to applying the hydrogel to the area.

76. The method according to 75, wherein the solution that disrupts the cross-linking in the hydrogel is water, citrate buffer solution, or an alginate lyase solution.

77. The method according to any one of claims 74 to 76, which results in the ciliate killing or affecting the fitness of the pest species.

78. The method according to claim 74 to 77, wherein the pest species is an invertebrate.

79. The method according to claim 78, wherein the pest species is a mollusc.

80. The method according to claim 79, wherein the mollusc is a Gastropod.

81. The method according to claim 80, wherein the Gastropod is a snail or slug.

82. A method of inducing the encystment of ciliate cells, the method comprising incubating a population of trophont ciliate cells in an aqueous solution comprising suspended soil particles, wherein the trophont ciliate cells undergo encystment to form one or more encysted ciliate cells.

83. The method according to claim 82, wherein the aqueous solution comprising suspended soil particles is buffered with a buffer solution to form a buffered aqueous solution comprising suspended soil particles.

84. The method according to claim 83, wherein the buffer solution is a HEPES buffer solution or a phosphate buffer solution.

85. The method according to any one of claims 82 to 84, wherein the soil particles are potting soil particles or pine bark particles.

86. The method according to any one of claims 82 to 85, wherein the soil particles have an average particle size of less than about 60 μm.

87. The method according to any one of claims 82 to 86, wherein the aqueous solution comprises about 0.01% w/v to about 0.1% w/v soil particles based on the total volume of the aqueous solution.

88. The method according to any one of claims 82 to 87, wherein the trophont ciliate cells are incubated in the aqueous solution comprising suspended soil particles at a temperature of about 20 to 30° C.

89. The method according to any one of claims 82 to 88, wherein the trophont ciliate cells are incubated in the aqueous solution comprising suspended soil particles for about 12 to 48 hours.

90. The method according to any one of claims 82 to 88, wherein the aqueous soil solution comprises magnesium ions.

91. The method according to claim 90, wherein the aqueous soil solution comprises magnesium sulfate.

92. The method according to claim 90 or 91, wherein the concentration of magnesium ions in the aqueous soil solution is about 15 μM to about 500 μM.

93. The method according to any one of claims 82 to 92, further comprising dehydrating the aqueous solution comprising the incubated trophont ciliate cells and suspended soil particles.

94. The method according to claim 93, wherein the aqueous solution comprising the incubated trophont ciliate cells and suspended soil particles is dehydrated to a relative humidity of less than about 80% compared to ambient humidity.

95. A method of stabilising encysted ciliate cells, the method comprising dehydrating an aqueous solution comprising a population of encysted ciliate cells and suspended soil particles.

96. The method according to claim 95, wherein the aqueous solution is dehydrated to a relative humidity of less than 80% compared to atmospheric humidity.

97. The method according to claim 95 or 96, wherein the aqueous solution comprising suspended soil particles is buffered with a buffer solution to form a buffered aqueous solution comprising suspended soil particles.

98. The method according to claim 97, wherein the buffer solution is a HEPES buffer solution or a phosphate buffer solution.

99. The method according to any one of claims 95 to 98, wherein the soil particles are potting soil particles or pine bark particles.

100. The method according to any one of claims 95 to 99, wherein the soil particles have an average particle size of less than about 60 μm.

101. The method according to any one of claims 95 to 100, wherein the aqueous solution comprises about 0.01% w/v to about 0.1% w/v soil particles based on the total volume of the aqueous solution.

102. The method according to any one of claims 95 to 101, wherein the trophont ciliate cells are incubated in the aqueous solution comprising suspended soil particles at a temperature of about 20 to 30° C.

103. The method according to any one of claims 95 to 102, wherein the trophont ciliate cells are incubated in the aqueous solution comprising suspended soil particles for about 12 to 48 hours.

104. The method according to any one of claims 95 to 103, wherein the aqueous soil solution comprises magnesium ions.

105. The method according to claim 104, wherein the aqueous soil solution comprises magnesium sulfate.

106. The method according to claim 104 or 105, wherein the concentration of magnesium ions in the aqueous soil solution is about 15 μM to about 500 μM.

107. A composition for stabilising encysted ciliate cells, the composition comprising encysted ciliate cells suspended in a buffer solution comprising magnesium ions.

108. The composition according to claim 107, wherein the buffer solution comprises magnesium sulfate.

109. The composition according to claim 107 or claim 108, wherein the buffer solution is a HEPES buffer solution or a phosphate buffer solution.

110. The composition according to any one of claims 107 to 109, wherein the buffer solution has a pH of about 6.0 to about 9.0.

111. The composition according to any one of claims 107 to 110, wherein the concentration of magnesium ions in the buffer solution is about 25 μM to about 100 μM.


SEQUENCE LISTING (9 sequences)

SEQ ID NO: 1 - DNA, 47235 bp, Tetrahymena rostrata strain TRAUS
atctattaatttccaataaataaacatatcaatatattatttactaaatagataacgtaagtgtgtttacgaaaacatttaaatcccaggcatccatctaatgctgttttttattaactactattatttaaagttgttaaaatttgaggtggaccggaatcgaaccggctatggttacccaatagatttacagtctaccgctttaaccgttcagccaccacctctttaaatatttatttaaatttacaggatttttaccttttaaaataatttttaaaaaaattttcaatttttttaagtaataaaactaataataataactcaccgttatattattctcaattgagtttattaaaataattactacgatgtttcgctttattattttaaatttaaatctattaatttccaataaataaacatatcaatatattatttactaaatagataacgtaagtgtgtttacgaaaacatttaaatccctggcatccatctaatgctgttttttattaactactattatttaaaattattttaaaaattaatataatatacttatgaccggggtcgaaccggcactccaaaggaatgcgattttaagtcgcacatgtctaccagttccatcacataagtaaaaaaaagcaaactgtagttaatattaaattataagtaaaawtttattcactaatgagagcttcacttaaataatttacttaaattaatttgtcttataaaaatttattagaaaacaatacaaatgagattcttacttatatataattcagtaattatcacgtttaatgtagcgttcaaaccatacccttattttattagagtaatttgtataccagtgattaaattttcctagtcctctcgtactaaagttaattttcgtttgttttcttttaattacaacagatagggaccaaactgtctcacgacgttttaaacccagctcacgtaccactttaaccggcgaacagccggacccttggtagattattcaccgccaggatgtgatgagccgacatcgaggtgccaaacgggtttattaataagaactctcaaaacccataagcctgttatccctagcgtaccttttatccgttgatcgatatattttctacaaaattatatcgggtcaatatgacagacaaaaatatccttaaaatacttgtatgtactttaatcaaacttagtaaattcattccaatattgatttaaattaaaattaaataccccaagtcattatactcctccgttactatttaggaggagaccgccccagtcaaactatctttctataaaataaataatataattattttaaatttatttttatttttaaaaaatatttaagtagttttccatttttagtaaatacctcctacttgtctagttaaatataagaaaaaaatattaatagataatagtaaaggtgcatagggtcttcccgtctagttgtaatattgccgcatcttcacggaaatttcaatttcgtagagcagatattagagacagcagggcagtcgttacatcgttcatgcaggacggaacttacccgccaaggaattttgctaccttaggaccgtcagggttacggccgccgtttactggggtttaaggttctagctaaaaaccagaacttgttcaccttgcagcaccgggcagatgtcagattcaatatgtcatatttaaatttagcagaatcttatgtttttattaaacagtcgctaccctctcttttttgttgcttaaagccccctttctcccgaagttacagggtaattttgcctagttcctttaatatcttttactctttcgcttttatatattctacttgtacacttgtgttagagttggtacgatttaaaaaatataaacatttccagcaactaatttattttttaaactatatactataataaatttaatatagtacataaaataaattattatgtattttatattttatattcatagataaaattaaacatttaaatcacagaaatcgacttacactaaataagattatcaaatttagtaacccttgtacattaagcgataatgattctcacattatgtctactactcatgctagcattatctttattgataaaatataaaacttcacaattttatttatttatacaaaatgttccactaccatctttcgatttatcaattcggtaagtaaaaattattctatccatttacgattatttttacatcaattatttaaaaacaataatattttctatattgagcttttacgctttcttaaaatgaaggctgcttccaagcctacatcttataggtcttattaaaaataatctcggtatttatttacttttgtagaccttattgtttaatctgagctgtttctctcttgactattaaccttagcgctaatagtctgattaatatttaatatttaaacaattattatatcctaattttataattaacaaattagctattaatatagttatttaaacctgtttaaaataaaaatatcaccctacttaaataggtttcgtggaaaaccagctataactaagtttgataagcctttcacccctaattacaatttatcccaaaattttgcaacatttaagagttcggtccttaggtaaatattacccttaacctaatcataattagatcacttagcttcgggtcttatataattaactattttttttttcaattcgtatttttattttaataaattactcgctaattatataaacttgctagcccattatgcaaaaggtacgttaaaatatattactttaactgtgtatagcattaaatttcagaatcttttaaaatatttaaaaatcttttcacctttccttcgcagtactagttcactataggatttaaatttttttagttttagaggtagatctctaatcttcaatttcaaaaaacatattgaaatttactaaattaaaattatatttatttattttattttttaatattttttaaattttatcgaaattagtataaataatataatgatgtgtaaaatataatttaaatatttaaatgcttgtaataaaattagtcttgacatacataaatttaatttaaattttatattattatatatataataatataaataatgttataaatattaaatacaaaattaatgatattatttgtataaaatatttagattcaaataattgtaaaaaatttattttatttgagttaaatatgaaactatctatagttttttttgttaaatattctatcataaatttttcaccgaaaaaaattgaactgtagataaaaatgtttctcataaacatttctgaaaaatttttaataataaaatctaaataaaaccctgattgaataaatttaaaaaatttaaattttattaatttattttttaacatccttgtaaattaatacatatattactttaaattcaaaatattaattaattttttttttctttcttctctttttttagaaaaaaaatcttcatgtttttcgattatgaagaaagtatataaaaataaaaataatcaaaatacaattgcgaaaaaagcaataaattgagaagaaactaaaaataaagtatgtaatatttcatatactcaatatataaattttccaggtaaaactgttaatatgtagtaagttgtagaatgattagaatttaattcattcatattatcagactgattttcgtttcctattaaaacattatttgttatttcaaattctgctacataatgtgataagcttaaaaaagttattaacattaaaacaattcttacccattgtgctaaaattctagtgtaaaacacaatacaagcgatgtaatcaaaaattacttctactaaactatttgtatttttacccgctccttttaagtaagctagaaaaaaaatacctaaatcgtataaaattagggttggcattcctaaaacaaaaataaacataataaatattgagtaatagctcattaatagggctgatttttcaacaattgtagttcaagcatgtatataaaaaaacaccccaaacacgtatgaaactataaatactaaccctaaataatcatctatggatgttatttctttttctgcttccactgttccgcttacagttaagtaatcagaatctattgtattttcttcagtattagcgttattataaaaactgaataaaaataaaacgaagcttgtaaataaaaaaaataatataattaaatgaagatataaagtatgggcaataatacaaatatctgggtttgatttatcaaaataaacaaattcaaatataccaccataaaaataagtaaaaaattgattaaatgaacttacgtgtcaagctaattctggatgatataatcaaattagagcattttctttagtgtgtaacacaaatctaaatcattcagcattaaatgggttgttttgtattaaaacactcatatatttatctaatattactgaataaaatagttgaaaaaatgatgttgataaggataatgttcaatattgattaaaaaataaatcaaattcattgtaatttgataaaccttctcataagtatcaaattttataatcgtaaaataaaatagattctgatgttgaataaattcaatctatgctatcaaaaacaattaaactaatatatgactcgtcagtactattaaaagtaccatctaatcatggaaacggcatgtgagtgggtatttctaaaataaaaggtttgtctagaaccattaatttaatttaatatacttattaataaatatattaatattaaactatttgttaaaattttaataaaatttatattagtttttttaaaatatttaaaagttataaaataagttctgaaaatatgtattaattctttaaacattaaattagtgtaaaatagagtcgaataaatttatatataaaagaaaattaattttatagaagaagaaaaaatttatatatatactgttataaaatactatttaaaaattttaagttgttaattttcaatttaataattttaataaatatattggtaaagtttttttatttaaataagaggtgtcttttcttacatttaagtgatttattagcataaatgtcattgttctaaaatctatttgaaaactttttgttaatttacttttgtagtgtagaattttttcaccaaacctagatattcttaatcttcttttatttttttctactttatttttcaatgtttttcaattatattttttatattttctcatactaatgtaaatttttttttttatttttttttttaatttaaatataaatttagaaaacattaattctatcatatctcctttatgaataaattttagtgggttggtttcttgctctccgtttaaaaacataaaaccaccttgaacaaacatgttagcatttctataagtataagcatatcttaattttaaagcaatgtttcttaatgagtattcatacatatgaaacctagttttaatatttaaggatgatatagatcttatatgtcttttaataaatttaaagctatgtttagatctatatttatgtattttataaatagatcggttattatctagatatcttatatattttttttttatgtcatgtagtctatctaattttttatatcgtctttgtttgttaaaaaaataatcaacatgagctaaattaatattcttgcttctatcaaaattatttttattttttttaaaatattttattattttttttatttttattttttttttaaaaaaacggcgtttagatagtctagctttaattttcgataggttaaacaatcttgtaaacctctttttaaatattaaaaaatattttaaaaaataaagtaatttatttatttttttttttttactatgtagtgtttttgttaaagtaattaattttaatttaggaaatttaaattttctttttcttttttttagttttagtttgtgtatttttatttttttagttaaattttttttaactaagaatagtatttttttatttttaattatttttaataaaaaagtaattattttattttcttctttaagtttattatttttataaaaataacaataaacattttttattataaaatagccatattttttatatattttttttagtgtgtttttagttttaattaattttaatcattttaaatacatttaaattaaaaatttttaatctcacgatgagtttttttttttttttttatctggttttttttttgatattatttttttttttttttgaatgattttagttttgttatcatcttcggttaatattattttaattttatttctttcttttgtacttaatgaattatttggttttaagtaatataaagtaaatccaggctcaaaacctaaagtaaatatattttttttttgttgagctttttttttttttgataatgctttttttctttcaaaaccatctatatatcctttagccatagaatttatgtcaattttaaacatactgtgttctttttgcattaattttaatcttttacttctagcttgtagtcattctttttttcattgtaattttcaaatataatttatctgttctgctaaatatattatatttagggttgagataggcatatttccgtaaattctttttgctatttctagtttaaattttcttaaatttaaattagatctataaacagatcaagcgtttgttcatgttctttgccccctaacaggtaaacctttagagtgtctcaaccccctatatgtatttaaataatctaaaaaaaatatattactttcaactctgttttttaaatttctacttttagggaaaaattttataattatggatttaaattttttccattctgcatctttatatgaaattatgttatctggtataaaagattcaaatctatcatatattaatttataagtatttatttttcagccataaattttattttttgtagaaaaaaaattagtatctttgttgaatatataaccatttagttgtaaaaaatctttcattttttaaaatttatatttatagttttctattttaaaagcatatttataagggttctttcttaaacatttaaataattttttatgtgtgcttaatattttatttttataatgatatttaaatcttttaactgtgaaaaaatttattagtacaataacccaattttgatattttaaaaataatatttttcctaaataataatgtctaagattataagattttttaattttacttatatttatgtgcttataacccctatttccacgatattcatcccctatttttcttatctttattagacaatagtagtataaattttcatttaaataatgatatataacgtcgtttaaaaataaaaaacttaagctatattttttgtaaagtttaatactatctcacacatttgttcaatacatagatatacctgatttatttaataatggtaaactttttagacccatttaattattatctcctaaaagattttgattttttttttgatttttttgggtaaacatgaggttttcttgttaaaataaattctccaaatttaaaaccaacatgaaaatccaatagttttaattctctatattttttgcccttgtgtataaaaaaaatattatctgttagtgaaaatggtatggtggatgctttattcataataatagagttttttttttttattttaacacctatagattcttttattattttagacatggttgatttcgctataaattttaaacgtcagtgtgatttactcatataattattatttatttttttttgttattcaacctcagggtgacatttctggttgatttgtttttgttctaccaccatgagggtgatctacaggattcatagctacacccctaactacaggtttaaaaccttttcatctatttatacccgctttacctattacaataaatttatttaaaaaatttgaatttctacctatagttgcttttgaaaaaccactaaaatattttttttttcctgtaggtaattttaaaataaatatattatgttcttttatttttttaattatacttaaataagtgcccgctgatttagcgtatttagaatttttgtcatttatagaaataatattagaaaaaacacaatatttaggtgctagatttaatatcataaaacaacctaatatgtttttaaaattaaatgagaagtttattggtctatctattgtttttgtaaaattacctataaataaaccatttgataatttaatataagatagtgaattatctgaatattttattaatccaataaaacatgagcttttattaatataatttaatgatgtaattaaagctagattagtggttgagcttctatttaaatttatactaatatatttattaaatttgttatgacttctagtacgaagtatagaagtaccatatttagctttacctgatttacttttatatttcatataaaataaattatttttttttataaaaggtgacattttatatatatcttttttgaatcttaaagatggtgttttgggtttatatttttttaaaaacatttgaaaattaattttaacaaatatatttgtttaattaaaatttgatattctcaaataattgagaaaaataaatttaaattatttaaaaaataatcatttacgttgttatttatttcaaaatttatatagttattattttctactaatattaaaaatttatataatttatttatattaatatcatcgtttttagcaataatgtgtttgtttttaaaaaaatttgatgaatatatatttttttgatataaattttgtacaactaaattatcgtattttaaatctaaaccgtgagttatggaatttacgtatatatatttttttattaataaattatttttaatttttaaatttaattttttaaaaataactgtattttttaaaaaatttgatatatatctagagaataacattagttttttaaattttttaaatttataatttcttttttttagaatatattttacttcttttttgtaatttaagaaaaattttttattagaaagtgatatcttttttattatattttttttcattttcattaatttgtctgtatagtgatttttgtaattgtaatacaccaaacattaaagcctcggctgttggtgggcatccaggaacatatatgtctacaggtattattctattacaacctcggacaacagaataagaatagtggtagtaaccaccaccgtttgcacaacttcccatagataaaactcatttgggatctaaggtttgttcatacagtcttctaaggccgggcgccattttatttgttaaagtacctgcaactaaaattaaatctgcttgtcttggtgtagctctgaatattaccccaaatctatcaaaatcatatctacttactgttgcgtgcatcatttcaacagcgcaacaagctaaaccaaaagttaatggtcaaaaagatccttgtcttgctcaggatacaacagcatttactggtgttactatataatctactctcatatatatatatatatatatatatattaaactctaaattttcttcttaatttttttttttcctcgtctggtttcggtactccgtatctagatcttctattaaacctatttaaaacttttgaaaaatcaaaaactcctctaatacaagtatatctaacaccaggtaaatctctagcgccaccgcctctaactagtacagacgagtgtcttcttatattatgccctattccaggtatatgagatgttagacgttttttgttcactagtattactttagctactggacgtctagctgagtttggttttcttggtgtaactattcttgctttcatgactacaccttttttttgtggattatcgccaagaattcttgctcttaattttctatgtaaattacctttgattttatttttgcataacgatcccaatataaaaataaaagaatacttgtttttttaatgtaaagagtgatattttatataaataaaaaagatgtgatataatttaaaattattaacacatcatttaaaaacaaaataccaaaaatatttaaaaaatttaatataactatatttaaaattatagaataatttatatttacataataatttttatagtttaaaattgatgacttatttttttttacagtaaagcgtagattttgaatataaaaatacataccgaaaatatttagtatagtcataaaaaatattattaaaaattgtgatttaaaagatgcgtaaagaatagctaaaaacttaccggtaaatcctagtaaaggaggtatacctgccatagataatatactaaaaattaatgtgtacagtataaaagtataagagttaaattctttaaattgatttaagcttttaaatttagaaaagtcacataataaaataattgaaattattaataaaaatactattaagtagtttataatatttaatatcaataatgataaaccgtatgaggttaaactattatcaattcaaatgttgtttaaatttgacatataatacatgaatagaagtatatttcaattatgaaaaccagagtctcctagaagtcagacaaacgcaaaacaaattaaaacaatgaatttaaattttggcccacaacatccagcagctcatggagttttaagattaatattacagttaaatggggaaatagcagagcgttttgacccacatattggtttattacatagaggtagtgaaaaattaattgaagatagaccttatttacaaggtatgccttattttgatagatttgactatgtttctatgatggttcaagaacacgcttattgcttaagtattgagtctttattaggtacaacaaattattctgctacatttactcaaattagaacaatgtatgatgaattaacaagaattttaaaccatttgttagctgttgcttgccatgctttggatgtaggaagtatgtcatcggttttttgagcttttgaagaaagagaaaaattaatggagttttatgaaagggttagcggtgctagaatgcatgcggctttttatagaccaaacgaagtaaatttaaatagtatatcatcttttttattagaggatattttagagtttggtagaaatttttttacatctttaaacgaaatgcataatgtattaacatataataaaatatgaaaacaaagattgataaatattggtacatattcttttaaaacatgtttggattatggtttaactggtgttatggctagatcatgtggtttaaaacgagatttacgtttaagtaaaacagaatcttatgctaattattattatctaaattttagaagttatgtgggtcagcatggtgattgttatgatagatttttaattagaatgaatgaaatgagcgaaagtgtgaatatagttaatcagtctatcaataaaatatcaaaatttaataatagtaaatcattaataaataatagatatttagttaatgataataattttaatagacacacttctattttacctcatttggttttaaaatatctaaataacaacgatttaaatttaaaaaacataaaaaatgactataattctatggaagaattaattaatcattttaaaatatgaagtaagggggttattgtcgagtctggttatacatatcaatcagtagaatcaccaaaaggtgaatttggtgttagtatggtgtctgatggtagtaacaaaccttataaatgtaaagtaaggtcaccagcattgcatcatttacaagtattacctaaaataggtaaaggtcattttttagcagatattgttgctttagttggtacagttgatattgtttttggtgaaatagatagataaaaataaatgttaataagacgtagaaaaaacactttaaaaagtaaatataaaaaaatatatatatatagaaataaattaaaaaatataattttaaaaagcttattttataacagaaatataaaaaccttaaatagatcttactcttatatgattttaaataacaataagatattatataaaaaatatcataaaatatgtaagtttaatggctacagaaaaaatgtaaataagtttacaggtattggtagacatgaattaaatagaaaaggtactttaggtcaattgcaaaatataagtttaaatagttgataatgaaggatgtaaatttttttgtaccatacaatattaattatttaattttaaaaaataaagaaaatatgtgtaacttcatatatttatataatgaagattattactttagtattaatgcatataataatattgtttttgtaaataaagatagtaatactattaaaattcaaaacatagttaataatgctaatactaatgtttttaatcaagaaataaataaatttttatttagttgagatagtattttttttaataaaataaaatttaccggtaaaggctttaaattaaagaaaaaaagtgagaacttgtttttattttttaatagagctcataaatgtttttttataggaaataacattattttaaaacgtttgagtaaaaataaagtactgcttttaaaaaataattataaacatttaacaaaagattcagttttagtgagaaaaataagagctaacaacatttttaccaaaagaggtttacgttttgcacgtcaaacaatttttaaaaaaaagggtaaaactgcggcttaataattaattaattataagatatcataaatctataacctaatacaccagtttttgtatgaattaatccttttttaaattttagttttaacattttattggttaagctatatttaccgcatcttatagcatatctttttttttttgaattacctccaccgcttattttaccgcttaaataaaaaaaaaaaccttcgaattttaaaacgtcataataatagttcaaggatttagatatgaataattttaaataatataaaaaacgtctatgatttttataatgcatagagttcataattcttaatagcactttcgcaaaaatatctatatcttttgtacataaaaatattaaagaaattcttaaaaaatttcttaatgatagttttgctcatcttaacattcttctcattcttctcatttttcttcgtaaaattttagcgtataaatttttttttttaaaaaatttaattgaatattgatcaaagtttattgatactttatgttttaatttactttctattaacttttttatagtaatatctactatagtcgttttatttaaaattttttcattagttgctttaaattgatctcttatatttaatatgctgtttatgttttttcttgtcatttgtcccttttttattatatttgatagagctaatgtatggattaaagttaaatcgtaactagtatgtattttattattcataaaatttcgatataaatacagtgtgtgattgtctattagtaatacagtattaataaaatttaaaaaatttatatcaaagtaatattttgacaagtatttatttgatgtaaatattatattattactgtttttatagattatcatcttaaataaattgtgattataattatagaactcttttatttttattgtgttaaaataatttgaataaaatttttcattagaaaaatttatagaattaatattatcaattttttttaataaatttattttttttataaaatttagttttttcattttaaattaaaaattaatattttagtctttttaaattttcatttttaaaatcagtatagtttaatcttgtagatttataaaaaccaaaaatagaatatgttttaaatattggttgtcatattataaaattactttctataagttttaaaatataaattattaatgagttaatataataagttttatatgaattcaataaaaatatattaaaagattttattttttgaaagttaaaaattataatgtgtttgaaagttgtatattttaatatatattgtttgaatctaaataaaattatttctattaacatattatgtgtgtataaaatagagtatgctattatatttttatttttttttatttttttaaaaaaaaagggaataaaagaatataatatataaaacaaaataacaaaaaaagtattttcatcttttccaaaaatttcatctctaagataggaacatataaaaaagttgttatctaaacttgtaataaaagaatatagaaataatcaaaaatataaaggtactatttttttgttttgtagtcttcaaaaatctttaaaaatacctaaaaacatcatattatattagtttagtacataatataatttatttttttttaaaagttgcctaagaatggaatcggaccaatgaccttcaggttttcagcctgacgctctaccactgagctacttaagcatagttttatagtttaaataaagaaggttacaaaaataatattaaacattaaaaaacctaatcatatatatataaagtgttttcagtttaattgtgtaaattgatcaaatctatatctaggtagtgttcctctagttgctatagcaacaacacaaaatattaatattttaaataataatacaaaatatggcattttttaattaattatattacgtaaacagcacctcctaaaaaaaaagataagaatacgaaagtttcaatgaaagcgaaacccattaaagtgttgttaaataaaccttcagattcttctgggtttctagatactgcaatattaaaaccagcaaataatacacctgttcctaaagcaccaaaagcaattggcatcatacatcctcctaatactaaagttttaactgcttttactaataacatttaattattttataaatccatagcttgttgatatatatacatttttcttgtatataaataagaatttttgtaactcaataatatgtctaaaaaaacattgttaattcttttttctaatttattttcttgttctttttttatacaaatagctatgtgtttattggctgtttttaatctatttttatccgctaagtatactattttaaataataagtcattatttgatttttttttatgtatttttggcacattaaaacatttaatatcaaaaactggcctataaataatatttattcagtttaaaacaaaatttaagttatatatttcattagtattagctacataatatttaaattgactggcgaaagtataattattaaaataactgatatcatatctgtttaatttaaataatatagaagaaattgctgtgactatattattaattgtttgtatttttttaccattttttattaaaaatggtataaaataggttataagagagtatttgtatttgtaattaaatctataattaatataaccacaatctaaatagtctaaaattgacatataactctcaaaagcatagtatttaccttcataaaatttatcacttgtagggtgtgatatataactaacatacattgatccttctgatccaacagaatttttttgaatttgtgggtttaacataatgttttcaaaaaaatttagattatttttaatatttttatgtggttttatttgatttatttctagtagattttttcttaaatgtttttgtaaacttatttgatttatgtaatttttttttaattgtttcaattttaaattattaaaaaattatagacaacaatagtaatataaaagatacactatttattattgtactataagctaaaaaggatttgaataaattaatatcaaataatattgatagtatataaaataaacctattatagatattataaatagtattattcaatagaaattgtttaaagatgacagataatatataaataataatgaaaaaaataaaaaaaatattaggaaataaaatgttgtgtagaagaatatagataaaaagggtagtcctttataaacttctattttatataattgtattggtgcaatacccaatttaattacaaaaccgattataaatactatacaaataattatagttgatatgtagtttttattataattaatttgactattagaagaaattatgaaattcataatagatcactcggttgttccggtgtatgtatagaaaaaaaaaaatgaaaatataattaagacagaactaaaaaatgacgatcaatattggtaaaataaaacatttaagtaatttttagaaaaaatagaaaaataattatcattagtatttttatttttaaaataaattttactaacaataaatttataaaaaattgaacaagaaactaactctattaaaaaaaaaaaagtaaataaagtattagctaaaaaaattaatggtataaataaagttatattaattattgagaatataaaatctacagagtatttattagagattttaatatgtttttcagttatgtataaaaaataattattaattagaataattaacatatataaatacaataaataattgttgattttaaagtgagatcatcaaaaagtggttgaatatccagagaaaacatataaatataaaaacatcatagaattaaatacaatatttactctaatcacaaatgatggattttgtcttacgattatgttttttacacttatggatttatctcaattattttttttatttaattgtgagagattatttttaaaaaaaattactcaataatttattaaatttattgttattggcaaaaataaaatataatcagatactaacagccctaacaaaataaaaataaaagataactggttatataaaatttcaattatatttataaaaaaatgaattattgctatttaaatttattatataagtttccataataaataaatagtatatattgctcaaatacatatataaccttaatggttgatattttttaaataaatttttaaatttatttttattttttataaaattattttttatattgtttatatcgtataagaaaattttttgtaatattttaaaattttaagtgtttaaatttattaaaaaatatttaataaaataggtatttttatgttccttgatatttgtgttactaaagttgattcaaaataattgtatgagtttagtaattttattattaaattatatagttcttttgaattatttattttaggtgttagatttgtttttatagagaaagataaagttaaataatatctattaacacctatactaaactgtgctattttatttttataaggagctctgagtatagaaccaacatgttttctagtttttattatatgtattttataatttaatggtttaaaatttttattaaactcattattaaataaatcaaaaactaatatagtgaatataaagtttttgcagtaaattctgttcgcttttattttatttagtacgatattagagtttaagattttagggctaaaattcattttatatcttatatttatttttatattgtttttcaattttaaaaaaattaaaaattaacttttgttataaaagctagcggtagtcctattttttttgatcattttttggttaatacgtttaatcttatatgattagtatttttaaactctataactttataacctctaggtaaacgaaccagtcatctatataaagttcctttacctttacccattcttgagtttttagattttttagttagtggtgtattcattctcataaaaattcaataatttttttttatatgttttgcgttttttttttttataatatttttaataaattttctaaatagttcgtagtatatagcctcaaatctagcatttttatatagtgaaattaaactattaccaaaatgaaaaatagtttttattgtttttctgtaaccccttcttttaaaagatacttgttttgtattacttaaatttttaaaacgcaaaattacatttttttttatatagtttaaatatatattatattttggtgtttattattttaatatattttacttatggtgtttttttttattttaattaaaattattgtataaaaaaaaaatataaaattgattaagtagttataagtatataaatatttattattattaaatatttcttctcaatatatatgaaatatttttgaataagatacttgtgtattatactcatcaataagtgagtttaaataatataatttatttgttattatattatttgatagataagtattagtattatctaatacgtttgttaatatatttttttgttttaatatattttcataaaataatatattttcataaaataatatattttcataaaattttaaatttaaaaggtagctaaacttaaaactaattattacataaaaatatataaacactataatgtgtaacattttataattttttatatttctataaatagaataaatatttatatttattataaaatcgttaaaaaaactaaaaataatatagtttatatttaaattgttatttttaagtttaatattataaaaaactaatcaaattaacaagaaacttatattaaatgtaattaaatttttataaaaaaatataatatttgataaatgagatacagtgttgctttttattaaaacacttaaaaacaaaatataaataaatttatagtttattttaaaacaattgtatgagttttttattttaattttttttttgtatttatttttaaatttaaaatacattataaatatatatattatataactgtacaataaatattggtttaaatttgttatactaataaaattatgtattgattgtactaaattatacctaactataaaaatataaaatataattacaaaggttatttgtgaaatgtataatttacagtatttaaaaaaccatattttattgttatggtatatatatataattattaaaaaatagtacaaatttattaactcaactaaatctcaatttcatcaagtacctcaatttaattcttggaaagctcatcaacttcctaagaaaatagcagaaactatgacataaaaatttgttttacatatttttttaaaaaattttattttttgtatttttgtatttttaaaaaagtttatataaatttttttattaaaatttaaatagtatttatataaaatattgctataaaatatatatattaatatggggtgtatcaaaaacaaaccatttaataaatttatgtttatatttaataaattttcaaatagtgtgtttgttttaaaacaatatatgtttactattaaaatagagtatattattattggtaagttaatttttttattatgtttttgatttattttttttttaaaaaaataaaatataataatatatatgttagtatttatatttgttaaattatttaatagttgtgtatatattgattcaattgtatagtttttaaataaaaaagtgctcaatattattaaaaaaataaatatatatttggttatatttttatttatatttataaagaaatataaattagttattatatagaatactgtaataataaaaatcataatcaattttattttaaaaattatttagtgcattttcatttttaattatgcgtttttttagtcttcttttaatagaccttatttttttatatttaaaaaaattatttggaattctcatttctataatattataaaaaataataaggtatttattacaaattttataaatttgttcttccattggaaaatattttttaaaatggtttattttaaaaattgttgttttttgtttaccaaatttttcaatattttcttgtaaaaaattatttaaatactctaaaaataaacgctcacctttattagatttttttttggctttttcgaagatttttaatgacgataaaattctaccaactgatatgttgtatattgttttattagttgattgtaaagttattcttaattgtctagattttaaacttagaactaatatgtttgggttctcataagcacttttattatttttattcattatttgttttataaaaacattatttatataaaaaagtttataccatgttgtgtttaaagtttcttcaaaattattattaacaagtaatctaaaagtcgatagaaaacctgaaataggttcagcaatattggaccctatgtaactagatgtgtataaaattttttctatttttatttttaacatgatatctaatgactggcttcaaaaaaaactttcgtgtaaaaagctatcatttttttcattaaattttttttttatcatggggtgtgtaatagggatcgaacctataacctttagatccacaatctaacgctctaccaattgagctatactcacctattattttattctattgatcatgatagtgcgttgtgtactcaatcatatcaaagagaagctactattaacattagaaaagtaaatattaaactaaattcaacaactgtaaacaaataatagttaaataacattggaaataaaaaagtaaattcaatgtcgtatagtattaaaaatactgccaacataaaaaaattaaaattaatttgtatatttaattcagacattgttttgaatccacattcataaaattgtttttttgtagcttgttgtttattggtgtaaaaaaattccgcacctcatgttagtaatcaaaaaattacacaaaagattaaaacattttgaattaaaataattacgatagcatcacccataaaattacaattttatcactacattataaaaagaaaatataaaaaataatggtagcatatatattctacaattataataatattttctagataaattacattaaaactagggtctagtttaaagccacctattttatcaaaaaatcagtcaaaaacttcgtatacactttgttcaactacgtttcaaaaaatagaatataagtaatgatatattagtacatatgataataataaaattaccaatttaactcaaatatacacacctcatccaccataacctataggtaaattatcacctataggattataaataatagagggtgttataattaaacttgttaaaagtttgtacttatttgctttttctattattataaattaatttcaatttattttttcaattggagattgaaaacttttatctaaaacagattgattattgtttttattatttgcatcaatatttaaagactcatttaagtttttatataatattgatattaaaattcctaatgaaaattctaaaccagcgacacctaaaataaaaaaagaggtgcttagtagagttatatcgcagtaaatagaacctaataatactgataaagtgtataaaactaatcatgttaattctgatataaataaaattgagataaaattttttggttttaatgatattaatattaaaactaatcaaaaaataatggatcaaaaggataatcatgttaacattttaaaatttataattctacagtttcattttttaaaattactacttgattttcaaatatattataaaaaacatcttgtgtaccctcgctttgaaattcttttagcattgggttttctattttagaatactctaataataattttctagtatcaatttttcatctgtaattaataccgtacatctcacttgtttctctttcaagtcaattagcatttggaaaaattctatccacagaatctatgtttttataaaaactatttattattaaaaatattgttaatttattttttaaaaaataattataataattataaaaaattaatagtttatttttaaaaaaataattttctattgagtttacagaaatgctattaaattttgtttcttgattataatgtgttgtatcaattgcagagttttctattagtgttatattagagaaaagtaattctttttttaagaataagttaattgaataaaatcaattactaggtataaggataacagaatgattggagtttaattttttagaggttcagatttttgaatttaatttttcaaaaacaataaatatttggattggctgcatatatttgtttatcatatttataaattatttttataacaagttataactttatcattcttcaaaattaggtcttcagtctaaaataatttcttccatttctttattttcaaaaatttttcaacctatttttttatattcacttaaaaaatctaatggaactaatactttaccacaactattttctagtcagaattttttaaatttttttaaatttttaagtcatttaattttttcattttggttatgtatgtttcgttttcattctctacatatttttttatatttcctacctctataaaaaataccatgatttagtattgtttcatctcatggattaatattatttgttttattatccaagctatcaatagcgtctaattcatcttccctattaataattcagtgagtatatatatgatccataaattttatatatgaatggcaaaatgttatgtaatttaattttgtactaaatttaaaaactgatggaaaaaaattaaaataaaaaaattttacattagatatgtatatcaacttgtcgaaaaaatacatatatggtttatcccatcaatttaaatttcaatgaaatatatttatttttttataattatgggttcagtcatattgtcacccataatagtcaaaaatattaaactccctaagcatattttcttttcttgacatagtttttgcaaaagctgtatataagaataacgtgtcttctttatctttttggtaggcgagtgttatttttttatttcataattcggatttgttgtatttattttggtatgcttgtttatagtttttaaattttaatcgcatagctagatatttttgttgatcattgtatagttgtagtggttgacatcaaatactaaaagctgtttgtggtcataaatctacaataaatttaaagtaccatgccattgttttttttggtattttatgtactcttcaatctaaatatataaaccatacaaaaaatagttgtggcccgtatcataatagtttaccatcatttatacctgtgtacgagtatgtaaaagtatatattaatatataggtgtaggctatgtattttaataaagtctcaaatgttaccatcgggaatgctacaaattttcaatccatttttacaaattttcggcaatatacaacgacggtgtataaagtaacaatagccattagtacgtcgattacaaaattaataatatataggttagtaatattcattaattgcaatatataatagtattgaaaataatttatttcgtatatatataacacagtaaagttcagtaaaaattgtataatactagtgtttattatttttaagaataaattaaatattattatgtcggtgttgtattttagatttatataaagtatatatacatgtatatgttttataatttttagtcttaagatttctattataaataatattaagatgtatttaatgtagtatcatctaggtgatttaaaattggcatgggtgtgtctaaatgcttcgtgctttttacaataatatataaatcaagctaggattaatatagttgagcagtaatatataattgtaatatttatattatagtttattgcttttagcataattacaaaatctattgtatttaaatttaaacaattaagcgctaatagtttggtgtaaagtattttatgtgtttctagaaaaaatggaaatacagtcattgttcataaatatttcccataaaaggagtgtgttttttttgaccaacttaatattattagtataccaattattgttgataaaacatataatatgtttttgtttaaaaatattatatctattataggatgtagctgtttatttagactaaaggtaaatatatagtttgatacttctttatttaaaaagtaagaaaatactaaaaaaatataggctgtttttttatgcagtatgtctattaaaagatgtaatattttttgaatgtttttttttagtattaaagtttttgtgtagtattttttaaagaatttatatttttttattaaaagtagttttattatttttgttatttttttatttttttttataaaaacagtgatagataaacaaacatcagataatagagtatttattagtgtgcttggtgctagtgatatcattaaaggttgtcttatagttaagtttgatagatttagttttttttttagttttttaaaattacatataaagtattttgcgtttttgatgttaatttttttatttataaaatagtttgttatatttatttttatattttttataaaatttaattgaattatgtttttattttttatagtagttaattctactatttttaggttatatatatatatattgtgtatttttaataaaattagtaatatattttgattcttttctaaaaatttatttaaatttaggagttgtggtgatttttttataaaaaggttcacaaatactatattcaaatttaaaatatggttattttgaattattttatttttagacattaatagtagtttagttaattttggtatctcatataataaatttaatgtgttaagtaatttaatacattgcgtttttacttgtttttttataattataattattttttttaaatataatttaaaatagtcgggtatttgtttttttatattattttttttaaaaagcttttttatatcttttttataaaaatttttattcaaaaccatataatattccctactatccagtaatctaattataattaattttaaataaaatattactattctatatgattttaagttatttttaatatatattaatatatacttattataaatatttatattatatatatatatagtaagtatttttattttttttaaaaaaaataatacgctattatatattattatatatatatatataatattttttaatatatatatatatatattatagttttttatatataagaagttattatataaatctaaaatttttaaatataaatacttcgaaataaaaaacaacctatatttaaatcaaagtgatttatgtaatctaattacattagttttttttttagctttagtatatatatatatatactgtataatattatgcgcatgaatactacaaattataatatagtgatttaattttgctaggtatggttttacaaaaactataatatatttataaaaaaaataaatattaaagtttaaaaaattaaaacatatatataaaaatgatctataaaaataaaacatgtatatgatataaaacaaaatatgttttgttgtttttttaaaaaaatatcataatcatttaattaaattggtaggtagattagttaaaaaagtgattaaattttctaaaaaattaaatttttttaagttatttaaaaaataatgataactttctctatctaataaaaaatctcaatctgccttaaaaacagctttaagtttgttataagattggagttcagttttataataactatcaaaacgttgttcttttgtaaaaaaaaaatttatacataataggttttttgaagttttattaaaataattttttgaatatagatagtttacgatagttacgtagattatattaaaatagctataaatttttaaaaataatttaataattttgtaaaacatatttataattatagtcggtgttgtttttagagaaatatatagatttttaataaatttataaaaattcaatctttcgtttgtatttttttgtgttgtttttgattttctataattagtatatgtttcttctaataaaaaaaaaatatccatgatagacgcagctattatgtaatattttttttttatataatgctcttctttcattattatataagcgcaaatataatcgataaaaaatcaagtatatgcaactgttcaaataaatttagttatgtaaaagctggtaattaaagaagctaataaatgtcaataaaaagaaaaaaaaaaggttgaataaaaaaaataaaaagtatacaattttaaaatatacatagataattctttattctcagggtttagcttatttttttgttttaatgctctgtagtttgtcagtatccaatttcatgaacccatttagttaaaataatgttttaaaaaataaaaataataactgttattttttaacagtttgtatttttttaaattatcaatattttaataaaaattttaatttaatattgaagtcgttaaaaataatgcaaattgggttataaattttttttttataaaatttttttttataaaaaattttttttggtgtacttacaacgaatttataataaaaatataattttttttaaaaaattgatacaaataattaaatttaaaataattaaatttaaaataattaaatttaaaataattaaatttaaaataattaaatttaaaataataattaattaaaaatagccgcaccaaaaagaaaaaaatcaaaacaactaaataaacttaaatatttaactaaattaaataaaataataaaaaataaaaaagtaaataaaataaaactacagttttaatataaactaataacatggaatgaaataaaggtatttatatatcaactaatataaaaaaaattattcaatattttgctgttatgacagtaagttttcacgatataaactctctatttgggtttttcacttttttaacaatagcaagtcaattggtaagcggtacaatgctagcttttagtttagtgcctgaacctatgttaatacctatggttagagaggaggaagatgtagaagatttatatacagacgattttttctgattacatgaaagaggggttgacatgctttttatattttcttattttcacttatttagaaaaatttatttaaacaactttgagtatgaacaagaagcagcttgaaaaagcggagtttttacatttttactatttcaagtcgttgtttttttaggtttagtcttatgttgtacacatttaagtgatattactttagctatagctgctaatttatacgatactttttttgcaggaaaaggtaagttttattgatgaatatttacatctaaagaattaaacaccgatactataataagactagcttatttacactatgttttagcattttttttatcgtacttaggtttaattcatggtgtagatatacattatgattgaaaaaatgaaaactcttatgatggtatagatacagaaatgatttgatttgacgaagctctttctaatgaattaggtggtatgatagaaataatattaattgtaatgattgtatgttttattgtatacccagaacctgaagccttgtcttacgaatattttatgtgaggtgacataggatttataaatgatgttagatttttatccgtagcaccacactgatattttagaccatttatggcatgattaacagtttgtccttttcataaaataggtttatttggtttaatttattatttcttcattttattttatcaaccagtaatacacggtacaaatgaacaaaataattattcaagaaaaaatgtagctataacaagctttattataaaaagaagtgatatattaacaccaaaatatcactccgttgaagataacatattacaccaaattacattttgactatttttatgtagtgctttatatgttacttcttacttaccttacggtagattttataatagaattaatggtaatttcggtacattatgatcttttatgtacttatttatctatttaggtaactcttttttaagaagacctttaattactgaattatttttttacagcacttttgtaaaatcaaaatttttaaaagaaaataactaaaccttgtaatttttcatttggtaaaaaatttaataaaaaattccattatatttttatgtaatactgtaagcaaaaaagaaataataacaaacgaggtgataaattttataaacaatattaaaatatttataaaaaaaaactatatgtacatgcttctaatattatcaatacatatttgagtaatagttagtatagaatattatcaatatattagaaatttaaatggaaataattgaacaaacatatacactacaggctcatattctaccaaaataacttttatatttttactgtttcctttatgactaataaacaccttacacaatatatatactatttttttaattacttgaactgaaatattattaagtgataaagaaatgttaagaaaagttataacaccccgtgatattattatacataaattatgatattatgatatgtttttaaatatagatacaaactcatttaaactaaaatatttatataatttcttaaataacttcagtattaaaaacactgcaaataatttttatgaatatcttgtatcatacccaatttttttattcacaggtttactttttttatttacaacacttttcagcctattatgtttaagttatttgggtttatatggagtttttgtattaaatttattatctatctctttattttgattttctatgattttttattttgacaatatcatgtctaataatggctattatctaataaatttaggtaaatgaatgtatctatcaagcggctataaagtttcttttgatctattaatagattcaacatctataagcttctcattcttaacattaactataggaatatttgtatatatttatacattctcctattttagatacgaaccattagtggagaggctaatgctttttttaaatttatttatgattagtatgattttacttgtatcctcaggtaattttattgtaatgtttcttggttgagaattaataggtttaacttcattttttttaataaacttttggtcaaccagggtcggtactttaaaagctgcgtttaaagccttttcatttaataaactaagtgatttatttttattttttgctatattattaatatttaacacaacatataatctcgatatattaacatttaactctcaaatatttttatatgagagttacactatagaaatatcgtatttttcaataaatttaattgaaataattagttttttttttataagttgtgcttttattaaatccgctcaactaggtgctcatatttgattaccagattctatggaagcacctgttccagcgtctgccttaatacattcagctacgttagtatctgcaggtatatacttattacttagactctcacctttatttgagttaagttattatgcttattatgttatacctacaattggatctctaacagctttttatggtggtatagtgtcagcttttcaatcagatacaaaaaaaactttagcgtactcaacaatcagccattgtggttttttaatggtaacttattcaacaggtgttttcgaatatgttatactgtatttatacgtacacggtttttttaaagcagctacattcttatgcatgggtaatgtaaaccgttttaatagaaacatacaagattttaaaagaatgggggggttttataaatacttaccttttgagtgttatagttcttttgtctgcatgataaatttaagtggtttacctttaacattaggtttttacataaaacatttactttttataggattaaacaattcttatataatatactacgtaatttatagctcattaattttaggtgccacagctggagtattttactcatatagattattttatagtattttttttgatgtcaaaaaaggtaaaaaaactatatatacacaagctaatagaaaaaatttaaactcaatatattacactaatacttcattagcctctaatatagccattatttctttagtagtaatctcatacataacaaccttatatttatataacacgactttaaataaattctatttattatctgatctaaaaacattaaatttaaataacggttattcttatttttatgcaccagataacaattttttaaatacaattagtattttaaattgatttattataatattaataatttctgttatttttttaaattgaaggcaaacatactactactcaacatctattgactctttaggcaaatttatcttattttcttattttttttatttaattacaaaatatattatataatttttaatttatatgtgaggaaatttatgaacagaagcatcttatcaactaaattttaatattggtttttcatctttacgttccgatgttcttattcatttagctcaatgacaatattgatgatgattttgatttgcattagtttgatctttttattattttataatattaagagtagttagatatagagttttaaaaatgagacctaaaattgctacaagttatagaccacacggtaaatgaggtgattttttagcttgtattataccattaatatgatgtattaatattttaactaattctaatttaatcttaagattaatagaatgacaaaatgaatctagtttattcacagtaagagttagagcaaggcaatgatattgaatatataaatttgaattaaaaaattttactgatattttatcagcccctaaaaatataggtaataatagatgacaaataaatacttttggtgagttacaaacagccgatgattatttacatgtcttacaattaagatcacaaaacaaatgagttaaaaactattgaaacaggtctttgcaagaaacaggtaaaactaacaaatctcatgtaatttctccacaagaacaactaagattaaatctaattaatcaatataaatcattaaatattttaaattctatttcaaacaacacaccgatgtttaactctaatctaaatatagataatgatattataaatataaatataaataatataacatcaaaaaaaacactgttaaatgataaaacatcacaaatagattccatttcttacttaaataatagttcttgatcaaattatgatatagatataagccataattacccatttttaaaagaatttgaatataatatatttaatgggagtacaataaatttgaataaaaaacaagttaatagtgactcaaaacaaatatttaaaaattttatatatgataaaccacaaaatagtgttatacaagattttacaaaattagtaaaacatgaagattttgatgaatactcaaggtgaataaaaagaagtcccggtgaaatattacctttaagaataataaaattcccaataggattagaaactataaatgataacaatgagcacttatttaaatttaggtttaataacaactcatcaaaattacaacctaaaatagttcaagatacaatatatctaactttaaaacaaaaaagatataatagaaaaaaaatagtatcccctcaagttaaatactataaagatgaaaacaacaataaaaccgatgtaactagatattctggtaaaccttatctatcaaatgacaaaatattaaaacaatcggtttatgacacaacaactcagtataaattaataaaaaaaaataaaaaaaggggtgaattaataccagtaacactagccagacgtttaattagaacaaaaaaaactttagtattaccagcccatgttaatattactataataacaaattcatacgatatcgtccactcgtgatttataccagggttaggtataaaactagattgtgtacctgggcgctcaactcaccacacattttttatagataatgttggattttattatggtcaatgcgcagaaatatgcggtagatatcatcatcatatgcctataagagtttgcgcacttccatttgaacacttcttaatatgatgaaatacttttggattaccaaaaatgttaaatactgtatctaaaaaaagatttgaaactcattatgaaatgagaaaatactcttgataattaatttatagaaaaaaggttgtatcatataattatattgagtgtgatcatggctcgggattaacgctaattagacgcctaacacatgcgagttttatgtaagttaaacttaatttagtttaaatatatagcgtcaaggtgagtacaatacaataatatgcctttaagtaaatttaaataatatatagtttttaaataacaaaaaaatgctaaaaacaatttaacactaatgataagtaaaaaaataaaatattcactataatctaaaattttaaaattttattgcagcttaaagattaatctgtataatattaggtaattatttctaaagttatttaataatttttttaattaaattaattatttttagtaaaataagccaatatgtttagttgaatttagagattgttcaaccacatatgggtatgaaaacgaccctatctttttgacagcagtgaggaattttggacaatatgcgaaagcatgatccagcgaactgatggaaatgaagaaggtatgtaatactgtaaagttttgtgcgagaaataacaaattgagtaaattttaggagaagctctggctaatacatgtgccagcagccgcggtaatacatgaggagcaagcgttatccacactgactgagtgtactaaatactacggtgattacaattagatttttgttaaaaggtataaaaactcaaaataaaaataaattcgtaaattctaatttttaataagaaaatggagacttatagaacagcgattaaatgcagttacactatatagcttgccagaaactaaggtgcttttctattaaaaattttagctgatgtatgaaagtatgggtatcgataaggattagtttccctagtagtccgtactgtaaaagatgaatactttatgatttttttaatcaggaatagttaacgcaaagtattccgctcggggagtattatcgcaagattaaaacttaactgaattggcgggaatttgttcgaacggtggaacatgtggtttaatgcgataatccacgcaaaatcttaccaacgtttaaggctttattagtagtataattaatattaaaactaattaataaagtacggaattgcatggctgtcgtcagttcgtgctgtgaagtttcggattaagtttttcttaacgaatgtaaccttataagtaagtttttaatggttattaactaaatataaactgttatttttatttaatctattttcaattaaaataaatttaattttgtttattttgaggataggggttataagctgaagtcaagtccctatggtctttatatgttgggctacacacgtgttacaagggtaaaaacaaagagacgcaatagagcaacctggagctaatctcgaaaaattatcttagttcagattgtttcatgaaattctgaagcatgaagatgaaatcgttagtaattgtaaattataatgttacagtgaaataatagtcaaattttgcacacaccgcccatcacgctcggaaagtcgatattggtggaagatttgaaaaagctttcaaaaaacaattaaaaaaattttttaaataaatttaattgttgtttttatgttattctttaacgttgtcagtaagagtagtatcgaatctagtattggtaatttgagtgaagttgacacaaggtactggtaggggaacctgttggtggaatacataattaaatcattttaaaaaaaaatataactaaataaaataattatatatttaaatttttattttatgcgattctagattattttattttattagacttaaatgacgattttgtaagaaaaacaatttttgaacaagtattaatttttttttttacttattgtgttatgaatttttttgcttgatctacagttatagagttaatttgacctactcactacttcaatagaagacatacatcatctactgaatttataaaattcagaacatatacagaagttttattaaaattatctgcgtataacgattttttctatatactaaataactattattttaatcaaaaattaatcttaaaaaattaaaaaccacatgttataccttaactgtagtttatatatgtcagcactgttattacattatatttgattagtattatatataattttttccataattttctctgtttttgtttttttattttatagaaaaaaaaaatcagacttaagtaaatataaaaacaactatttttcaaaaattattttaattttttttgcaaataaaaatttaaatctatttaatcaatatttaaccacctctgaaataaactttatagaaatagaaagatcaaggaattgacctaccttaaattttatacactcaaatataaataaaataaataaaaataacaaaaacatttttgataaaaaatcttttaaaaactattacaacacttttaattttttaaaagaaaataaaataaatttaaatattgaaaacagtgaaaattttttattaaacttctctataatcataaatatactaaaattaaaatacaaaaaattttattatctaaatatattagatttactagtgttattactattagctaaaaactcaatatcaaataaaaaattcaacatatcaatatttaatacaaattatttcaatgattatgttaattatataaaattaacaaatatcgataacactaataaaaactcatatttattagttataaatggtaacaccaatcgcacaattagcacaaaatttaaacctattgtaattaaatcaaataacgttttcaataatttaaataattttatttataacactaattctaacaaaaatctattaataaatttaaacgaaaattttattaataatttaaaaaaaatatttaaaatttctttctcatcatctaatattacaaaatatatatctaactatacaaccaacaattctattatattatatttaagaaaaaataaaattttcaataaaagcagatattctagaaatagacaaacttatagaaccggtgcttactgatgtctatatgtaaacataatagctgtaatagctttttatttttgattttataaatttacatttaattttggttacgtgtgatgaatactatatatatttattttaagtttttttttacctagagcattaaaatataaattctataatcctataaaaatattaaacgaaattaaattaagtttagtttgattatacactatttttaaaaatattttaaactttaattttaaatacttaaatataatttttatctgaatgtataaatctattttagtaaaaagtaaccaattattttttataactaaattagcaaaaataaataaaatagattttatttatatttgacaatcattcaatttcattaaacaaactaataacttaatactatttatatgaaataaaatataaataatagagttaatttaaaatagggggaatagtttaatggtagaacaacggtcttcaaaatcgttagcgtgggttcgatacccgcttccctcgttttataggtaatgtaattttttaatggatagaaaaaatttaaaaacatactgatttgattttaacggagcaattaatgaacaactacctctttcttataatattttaaagatttgtaaaaaagaaattgataacatggagaaaaataacaactattttgtaagtactaaagaaaaaccaattaaaatacaattaaactttaatcaaaaatataatacagagtgtgttagtgaaattgataataatatgtatgaaaaatctaataaaaatagttttttatctaatataggcaatcatttatactctctaattaatttagttatacaaccaataaatatactattagagtttaaattaaactatttatcatcaaaattaaacataaaacactactatataattaacaataaattatacataacatataaaaactcgttaatattatttaattcacacaatgactatattaatgatattaaaaaaataaataatgtaaaatacaattttatttatagaaatagaattaacataaatacttctttttatgactttattgatacaatgtttataaaaataatattatatacacatttaaattacttaaaaataaaacacaacaatagattaaattatcgtttaaaacaaactgattgaggtttttatataaaaaacaataataatattattaataatttaataggaggtattaaatttatttgaaaaggtttaagattttgattaataggtttattattaggtgtagcaataatttattatttaatgtatattagattactaccttttaataagataatattatcttgaattttagtagcaatgtttttatattgattgatatctggttttgttttttttattaaaaaataccaatacagtagatttacagcagctatacaacgtttttgaaaaagaacatacataattttttgattaatagaggctggtactttttcagtatttttttatttaactttaaactcatcttctgaacctgtttatatgtacgatcaaattaaaatatttaaaaataatttattttcttgacgttgatttttaataaaactactaccgtcagtttctattatattacttggttattatttacaaataacattaaaatgaaatttatttaataaacaaagtacaatattatttttaataactataatattattatatatattgtgacttgagttttaccaattttttcacatattaagtttttatggaaatataagttgattatatgattacgatgagtatatttgaactttagagatagatacacgtagaacaagattagctaataattatatttctatttgtctattcgctaaattttgacattttgtttttataactgtgttttgaatattttttgttttaagaataaacgaattagggagaataagataccctttattagttgctaatgtgcaaaattttataattatttatattatgtcttgagcttatatgtacccttgactaaaatttatatttagaaaatatttagatgtaccttattattgattttttactaatggtagaaaattaggtattagagtattttttacagatttaaaactatttttttacggaattacaagcaatttatttaactttaatataaactttataaaatttgataagtatccattctattattgaattaatgcgtcaaatatgacagactttaatcaatatagaaaatttataataagagattctattatacatagtttaaataattatgttatctaatataaaatttttaagtgtttaattttttattaattttttttataaatctattattattaataaatttttttttttttgaaattttaataaaaaaccatattattgtttattatgtaacaatcctatattgcttattaatattactactagtttttttcaaaaaaaaaaaaaaaatgctaaaaaaaaataattactatatttttttaataattttttttttttgaaatctaaataaacaaatttttttttttaattatagtaatattttatatttatgtaaatatatatatatataagcgtatttgtattatgtgagttgattttatagagcagactaaaagttttaaagtctcaataaataattatttttattacttaaacagaataaaaaaattatttacatattttaacgatttaagaaaacatgtcttaaaaaaatatgtttatacaattaatcataaaagaatcgctattaattatttatattttagtatggttacaggtttatctggtgcagctttagctactatgataagactggagttagcccaccccggaagcccattttttaaaggagattctttaagatatttacaagtaattacagcgcatggtttaattatggttttttttgtagttgtacctattttgtttggtggtttcgctaattttttgataccttatcatgttggttcaaaagatgtagcatatcctagacttaatagtattggtttttgaatacaaccttgcggttacatattattagctaaaataggttttttaagaccccagtattgaagatattatgataaaacatccttctcttttccatttttagaaaaattaaaatataatcaatacaaagaatataaaaatgattatttattttatcttgattttttaaaaaaagaaataactgatgatcattcttttttctgaaaagctagaaaaacaataaaattacctcaatactcagttttttcttttgtacctttaaaacttatgatgtgaaaaacaataattaattatccagaatctttttgatatgctgctagtagagttgttcaatctagacgtaaaaaagtttttgtaacaaaatgttcagcgagaactttaacaaccgctggttgaacttttattacaccattcagttctaatttaaaatacacaggtgtagggtctcaagatgttctaattttatcagttgtttttgcaggtattagtaccactatatctttcactaatttattaataacaagaagaactctcgcaatgcctggtatgcgtcatagaagagtattaatgccttttgtaactatatctttatttttaactttaaggatgttagctacaattacaccagtattaggagctgcagtaataatgatggcttttgatagacattgacaaactacttttttcgaatatgcttatggaggtgatccaatcttatcacaacatttattttgatttttcggacaccctgaggtatacgtacttattatacctacatttggatttataaatatgataatacctcacaacaacactagaagagcggcttctaaacatcacatgatttgagctatttatgtaatggcatacatgggttatctagtttgaggtcatcacatgtacttagtaggtctagatcacagaagcagaacaatgtatagtactattactatcatgatttctatgccagcaactattaaagttgtaaattgaactttatctttagtaaatggggctttaaaaatagacctaccttttttattttctatctcatttttattattatttttagtagctggtttcactggtatgtgattatcacacgttagtttaaatgtctcaatgcacgatactttctacgtagttgctcattttcacataatgttatctggtgctgcagtaacttctatattctcaggtttttattattactttaacgctttatttggaataaaattttcaagaatgtttgggtacatgcacttaatatattattctggtggacaatgagtaacttttgtaccacaattttatctaggtttttctggtatgcctagaagaatacatgattaccctgcagtatttatgggttgacatagtatgtccacaacaggtcattttataactttaataggtataatgttctttttcttaatgatatttgattcacatattgaacgtagagcatcaacatctactacattaggtttacctagatgatacaaaagaatatcatactatatctttaaaattcgttacttacaacataataaaacaaaaatgaatggtgtaccaggatccacagttagattaatgttaataaatagacattttacagaattcgaaatttataaaaaataatttaattaaatgagttattgaatcgcgccattatttatcaatcttttatctattccgttatacttggtaattattctatataatattgtttgtatgctgttaataactttggtaattgcttcaattacattaatagaacgaaaagtattaagtttaatacaaagaagagttggcccccactatgtaggttataggggtagattacaatatatagccgacgcactaaagttatttataaaaggtgttattataccagaggggtcaaataaattttggtttgttactataccatctatggctggtgcagtttgttatactttttgaatgaattcaatgtgaggacctagtgtttcaatttttgaaattgaatataatatggtgtacgccactttattatctatattgtttagttattgtattatgttaacaggttattttagcaaaagtaaatatgctttcatggcttctataagatgtgctgtattaatgctaaatatcgaaatatttttagggttgttagttataaatttagtttttgtaacagaatctttttgtttctcagtttttgtaatatatcaagaaattatttgattaatttttatatttataggtgtatcaggattaatttttataacttttttattagaaacaaatcgtgccccatttgatttagctgaagctgaatctgaattagtaacaggttatagtgttgaatacggtggtttttattttgctttatattatttgggtgaatattttcatttgttttttttttcaatggtcttaagtattgttttatttggtgggtgagaattacctaattttatatattattttatcataaatgatttttttttagtttaataaactaaatgttataactttagtttaaatgtttttaattacttttacatcaatattttcaaacattatagtcttcaataattttattatatctttaattgatattttaacacctatattttttattgaaaattttattttacagttttttataacatatattttatatttattggttgtaaataacaatttatattatatactactatatgttttcctagaaatagttttttttggtttatttatttgtttatatcaattagaattatttacaggttttttatgagtagcagaatttacaataatttttatcagtgttgttctactcttttacttaaatgttgatggtttacatctaaaatacaataatataaataatttattttattatttaccatctattattttatttttattagtttattgcttagattttttatcagaactagagttatacttaccaatagaatttaattatatggatttttatgatgactattatgaatcttttaacaacactaatatgaatgattttataccactaacactaagttattattctgtaaatggtattgaatttatattaataggattactattattattgggtagtgtagcttgtgtaaatctttataaatctaataaaaatttttctataataaaacagtctaatttactaaatatgttcaatttttttaaagattttataaacttttcatttttaagaaaacaagatttaaattatcaaactaattttaaacctagcttaagatctataaaaaaaaaataatttaatgatacaaaaagaaactaatttaaaaccaatagataaatgtggtgtgtggtctgttcgagcctttcatttatatggaggttcttttaggcaacactcatcagtaagtaattttatcaaaataagtgtaaaaaaaactcgcgcaaataactgaatacctaaaaaaactaaattaaaagctattatagtaacagtaaaaaaagaaaaattaaaaatagacggtagttatataaaatttagatctaataatgtagttcttttaaaaaaaagattaacccccaaaggtaaaatattgatagggcctgttagcatcgccttaaaaagaaaaagatttttatcatctttttgtgccttcatataattacatataaaatttataactcttatcgtctaatggtcaggacagcgggttttcaccccgtaaatctgggttcgaatcccagtaagagtacttatgtttagatgactttttttatattgatacaacactactgataccccatactcaatctctaaaatcaatctttgatcttatattaatataagattatttaaagctaaaattagcagcagtatagcatattatatacttagcttaaataatttggaattaaaaaaattaaaaattttttataaaaacaattattttgattatatttatttaaaatcaataccttgttttttttttatttttttattattaaatttttatttatttatttaaatatttaataataataaccaggtaggtatgttaaaaaaaaccccattaggtatgtctatagatattccactagaaaataattttctatatacattatactcatcaaaaattttcatagaacttaattttttttttttattaattttattatttatatcttttattatttttaatttatcaaaaattaattctattaaaaaaataaacaacgctaattctgtatttacagcaataaaacttataaccattattggtttattaatttctttttttatgcatattttatgttttttagcatattgtttacattcatatcattattctttagaaattttttctcgtacaagcttaataaattcttattcaatcgaaattactaatgtaaaatgagataaatatttacctagatactttaaaacaaatataacaatcgatttttttggtttaatactattaactcttgcttatattgtcggttttatatctattttagcgttagacactagattgtattgaaaaaatattaaatatatattttcttttaatatttttttgttaatcgtatatgtttatgtatcagtgtcaaatttatttttatttttcatgagttacgaattattgttaataccttctttttttattgtatattttgttagtcctagtcgtagagctatacaagcttctttatattttgttatatgaactcaacttggctcattattagtactaatagctatttcctatatagtttctttatctaatactagtgaattttcagatttaaaatattttaattttacaacaaatgagtcaacaataattgttttattacttttttttggttttggtttcaaagtacctatttgacctttccactattgattaactaaaacacatgtagaagcacctagtggtttttcaatttatttgagtggttttttagttaaaacagctttatatggtttttataaattaaatacttctatttttatagatatgaattcatctgtttttattgctatttgtataatgggtgtggtagactcttctttaaaaatgtgaggacaaactgatttaaaaaaattggtagcttacggtactatacaagaaatgaatataatatatttggctttttgttgaggtgattcaacagctattctaggtggtattttatttagtgcaactcacgcatttttatctgcgttaatgttttttttagtagattgtatttacagaagatttcatactagatctctagtcgaaataaatggtttattacatgtcacacctaacttaggtttatctatattgtttatgttagtttttttttcaggtatacctggaactataaaatttataacagaattttatatatttaatggtttattagaagcttcaccagcatcttgcttaatattaatgtttgtagcaaatgtattaggtttaataggatttagcaaagcttgatttaatgttgtttttggcatgcctaaaaaaaatacaaaatttctaccaatggatctatcatttaaagaatcttatattattttatattgttatttttttttatttatattttcttatttttcatatatatttttttaaattttatgaaaaaattaatttataataataaataaaaaattaaacttatttttaatagaaaataaaaaaaacttaaataacaaaaatttaaaaaataaattaaaccttaatattaattatataaaatatttaaatttaattaattttaaagaactaaaagcattaaatagtttattaaggtgtataattttagtaaataaaataaaaaaaactgttttagtttataataacaactttatatctatattatatagaagtaatttttataacagattaattacatataaatttaataacactgaattagactatatttataaaatatttagttttacaaatgtatctgtttttgtaaatgcatctagtaaatatgtaaaatttaaagctgaacatgaaagaaacataaatttttcattagactgttttcataataatatgcctagaaacccagcgcattacttagttggaaaaatgtatgtgttagtaatgtattatttaatttaaatttactcataagtaatatataattttaatttagtaaatttcaatatgttttttgaaattgaagattagagatctacctctaaaactaaaaaaatttaaatcctatagtgaactagtactgcgaaggaaaggtgaaaagatttttaaatattttaaaagattctgaaatttaatgctatacacagttaaagtaatatattttaacgtaccttttgcataatgggctagcaagtttatataattagcgagtaatttattaaaataaaaatacgaattgaaaaaaaaaatagttaattatataagacccgaagctaagtgatctaattatgattaggttaagggtaatatttacctaaggaccgaactcttaaatgttgcaaaattttgggataaattgtaattaggggtgaaaggcttatcaaacttagttatagctggttttccacgaaacctatttaagtagggtgatatttttattttaaacaggtttaaataactatattaatagctaatttgttaattataaaattaggatataataattgtttaaatattaaatattaatcagactattagcgctaaggttaatagtcaagagagaaacagctcagattaaacaataaggtctacaaaagtaaataaataccgagattatttttaataagacctataagatgtaggcttggaagcagccttcattttaagaaagcgtaaaagctcaatatagaaaatattattgtttttaaataattgatgtaaaaataatcgtaaatggatagaataatttttacttaccgaattgataaatcgaaagatggtagtggaacattttgtataaataaataaaattgtgaagttttatattttatcaataaagataatgctagcatgagtagtagacataatgtgagaatcattatcgcttaatgtacaagggttactaaatttgataatcttatttagtgtaagtcgatttctgtgatttaaatgtttaattttatctatgaatataaaatataaaatacataataatttattttatgtactatattaaatttattatagtatatagtttaaaaaataaattagttgctggaaatgtttatattttttaaatcgtaccaactctaacacaagtgtacaagtagaatatataaaagcgaaagagtaaaagatattaaaggaactaggcaaaattaccctgtaacttcgggagaaagggggctttaagcaacaaaaaagagagggtagcgactgtttaataaaaacataagattctgctaaatttaaatatgacatattgaatctgacatctgcccggtgctgcaaggtgaacaagttctggtttttagctagaaccttaaaccccagtaaacggcggccgtaaccctgacggtcctaaggtagcaaaattccttggcgggtaagttccgtcctgcatgaacgatgtaacgactgccctgctgtctctaatatctgctctacgaaattgaaatttccgtgaagatgcggcaatattacaactagacgggaagaccctatgcacctttactattatctattaatatttttttcttatatttaactagacaagtaggaggtatttactaaaaatggaaaactacttaaatattttttaaaaataaaaataaatttaaaataattatattatttattttatagaaagatagtttgactggggcggtctcctcctaaatagtaacggaggagtataatgacttggggtatttaattttaatttaaatcaatattggaatgaatttactaagtttgattaaagtacatacaagtattttaaggatatttttgtctgtcatattgacccgatataattttgtagaaaatatatcgatcaacggataaaaggtacgctagggataacaggcttatgggttttgagagttcttattaataaacccgtttggcacctcgatgtcggctcatcacatcctggcggtgaataatctaccaagggtccggctgttcgccggttaaagtggtacgtgagctgggtttaaaacgtcgtgagacagtttggtccctatctgttgtaattaaaagaaaacaaacgaaaattaactttagtacgagaggactaggaaaatttaatcactggtatacaaattactctaataaaataagggtatggtttgaacgctacattaaacgtgataattactgaattatatataagtaagaatctcatttgtattgttttctaataaatttttataagacaaattaatttaagtaaattatttaagtgaagctctcattagtgaataaawttttacttataatttaatattaactacagtttgcttttttttacttatgtgatggaactggtagacatgtgcgacttaaaatcgcattcctttggagtgccggttcgaccccggtcataagtatattaymtyaawttttaaaayaactttaaataatagtagttaataaaaaacagcattagatggatgcctgggatttaaatgttttcgtaaacacacttacgttatctatttagtaaataatatattgatatgtttatttattggaaattaatagatttaaatttaaaataataaagcgaaacatcgtagtaattattttaataaactcaattgagaataatataacggtgagttattattattagttttatttaaacaaattatttaaaattttataattaaattatcatagtaggtaaaaatccagttaataaaaataaaataatagctgcttggatggttccagtacgggctcatttcccaataccaaaaagttcgattcttttaagtggccaatttatattaaataaatttatacttttatttaattacacccccagatctagtagtaccaactacccagacaccaatacacccccagatctagtagtaccaactacccagacaccaatacacccccagatctagtagtaccaactacccagacaccaatac

SEQ ID NO: 2 - DNA, 689 bp, Tetrahymena rostrata strain TR 1034
atgataagactagagttagctcaccctgggagcccattttttaaaggagattctttaagatatttgcaagtaattacagcacacggtttaattatggttttttttgtagtagtacctattttgtttggtggtttcgctaattttttgataccttatcatgttggttcaaaagatgtagcatatcctagacttaatagtattggtttttgaatacaaccttgtggttatatattattagctaagataggttttttaagacctcagtattgaagatattacgataaaacatctttttctttcccatttttagaaaaactaaaatataatcaatataaagaatataaaaatgattatttattttatcttgattttttaaaaaaagaaataaccgacgatcattcttttttctgaaaagctagaaaaacaataaagttacctcaatattcagttttttcttttgtacctttaaaacttatgatgtgaaaaacaataattaattaccctgaatctttttgatatgctgctagtagagttgttcaatctagacgtaaaaaagtttttgtaacaaaatgttcagcaagaactttaacaaccgccggatgaacttttattacaccattcagttctaatttaaaatacacaggtgtaggttctcaagatgttttaattttatcagttgtttttgcagg

SEQ ID NO: 3 - DNA, 689 bp, Tetrahymena rostrata strain TR 1035; identical to SEQ ID NO: 2

SEQ ID NO: 4 - DNA, 689 bp, Tetrahymena rostrata strain TRO1
atgataagactagagttagctcaccctgggagcccattttttaaaggagattctttaagatatttgcaagtaattacagcacacggtttaattatggttttttttgtagtagtacctattttgtttggtggtttcgctaattttttgataccttatcatgttggttcaaaagatgtagcatatcctagacttaatagtattggtttttgaatacaaccttgtggttatatattattagctaagataggttttttaagacctcagtattgaagatattacgataaaacatctttttctttcccatttttagaaaaactaaaatataatcaatataaagaatataaaaatgattatttattttatcttgattttttaaaaaaagaaataaccgacgatcattcttttttctgaaaagctagaaaaataataaagttacctcaatattcagttttttcttttgtacctttaaaacttatgatgtgaaaaacaataattaattaccctgaatctttttgatatgctgctagtagagttgttcaatctagacgtaaaaaagtttttgtaacaaaatgttcagcaagaactttaacaaccgccggatgaacttttattacaccattcagttctaatttaaaatacacaggtgtaggttctcaagatgttttaattttatcagttgtttttgcagg

SEQ ID NO: 5 - DNA, 689 bp, Tetrahymena rostrata strain TRO3; identical to SEQ ID NO: 4

SEQ ID NO: 6 - DNA, 689 bp, Tetrahymena rostrata strain TRO2; identical to SEQ ID NO: 4

SEQ ID NO: 7 - DNA, 689 bp, Tetrahymena rostrata strain TRAUS
atgataagactggagttagcccaccccggaagcccattttttaaaggagattctttaagatatttacaagtaattacagcgcatggtttaattatggttttttttgtagttgtacctattttgtttggtggtttcgctaattttttgataccttatcatgttggttcaaaagatgtagcatatcctagacttaatagtattggtttttgaatacaaccttgcggttacatattattagctaaaataggttttttaagaccccagtattgaagatattatgataaaacatccttctcttttccatttttagaaaaattaaaatataatcaatacaaagaatataaaaatgattatttattttatcttgattttttaaaaaaagaaataactgatgatcattcttttttctgaaaagctagaaaaacaataaaattacctcaatactcagttttttcttttgtacctttaaaacttatgatgtgaaaaacaataattaattatccagaatctttttgatatgctgctagtagagttgttcaatctagacgtaaaaaagtttttgtaacaaaatgttcagcgagaactttaacaaccgctggttgaacttttattacaccattcagttctaatttaaaatacacaggtgtagggtctcaagatgttctaattttatcagttgtttttgcagg

SEQ ID NO: 8 - DNA, 689 bp, Tetrahymena rostrata strain 1015
atgataagactggaattagcccaccccggaagcccattttttaaaggagattctttaagatatttacaagtaattacagcgcacggtttaattatggttttttttgtagttgtacctattttgtttggtggtttcgctaattttttgataccttatcatgttggttcaaaagatgtagcatatcctagacttaatagtattggtttttgaatacaaccttgcggttacatattattagctaaaataggttttttaagacctcagtattgaagatattatgataaaacatccttttcttttccatttttagaaaaattaaaatataaccaatacaaagaatataaaaatgattatttattttatcttgattttttaaaaaaagaaataaccgatgatcattcttttttctgaaaagctagaaaaacaataaagttacctcaatactcagttttttcttttgtacctttaaaacttatgatgtgaaaaacaataattaattatccagaatctttttgatatgctgctagtagagttgttcaatctagacgtaaaaaagtttttgtaacaaaatgttcagcaagaactttaacaactgctggttgaacttttattacaccattcagttctaatttaaaatacacaggtgtagggtctcaagatgttctaattttatcagttgtttttgcagg

SEQ ID NO: 9 - DNA, 691 bp, Tetrahymena rostrata strain 1016
atgataagactggaattagcccaccccggaagcccattttttaaaggagattctttaagatatttacaagtaattacagcgcacggtttaattatggttttttttgtagttgtacctattttgtttggtggtttcgctaattttttgataccttatcatgttggttcaaaagatgtagcatatcctagacttaatagtattggtttttgaatacaaccttgcggttacatattattagctaaaataggttttttaagacctcagtattgaagatattatgataaaacatccttttcttttccatttttagaaaaattaaaatataaccaatacaaagaatataaaaatgattatttattttatcttgattttttaaaaaaagaaataaccgatgatcattcttttttctgaaaagctagaaaaacaataaagttacctcaatactcagttttttcttttgtacctttaaaacttatgatgtgaaaaacaataattaattatccagaatctttttgatatgctgctagtagagttgttcaatctagacgtaaaaaagtttttgtaacaaaatgttcagcaagaactttaacaactgctggttrtgaacttttattacaccattcagttctaatttaaaatacacaggtgtagggtctcaagatgttctaattttatcagttgtttttgcagg